(19) United States
(12) Patent Application Publication
(10) Pub. No.: US 2009/0105278 A1
(43) Pub. Date: Apr. 23, 2009

(54) Selective inhibitors of human corticosteroid syntheses

(75) Inventors: Rolf Hartmann, Saarbrucken (DE); Sarah Ulmschneider, Mainz (DE); Ursula Muller-Viera, Ingbert (DE); Rita Bernhardt, Saarbrucken (DE); Mattas Bureik, Saarbrucken (DE)

Correspondence Address: Ballard Spahr Andrews & Ingersoll, LLP, SUITE 1000, 999 PEACHTREE STREET, ATLANTA, GA, 30309-3915, US

(73) Assignee: Universitat Des Saarlandes, Saarbrucken (DE)

(21) Appl. No.: 11/658,048

(22) PCT Filed: Jul. 21, 2005
(86) PCT No.: PCT/EP05/53561

§ 371 Date: Sep. 14, 2008

(30) Foreign Application Priority Data
Jul. 21, 2004 (DE) 10 2004 035 322.0

(51) Int. Cl. A61K 31/505; C07D 213/06; C07D 213/26; C07D 213/30; C07D 215/12; C07D 217/02; A61K 31/472; A61P 3/10; A61K 31/47; A61K 31/426; C07D 239/26; C07D 277/22; A61P 9/00; A61K 31/44

(52) U.S. Cl. 514/256; 546/348; 546/346; 546/343; 546/152; 546/139; 544/242; 548/202; 514/277; 514/365; 514/307; 514/311

(57) ABSTRACT

The invention relates to compounds for selectively inhibiting human corticosteroid syntheses CYP11B1 and CYP11B2, to the production thereof and to their use for treating hypercortisolism and diabetes mellitus or insufficiency of the heart and myocardial fibrosis.

The invention relates to compounds for the selective inhibition of human corticoid synthases CYP11B1 and CYP11B2, the preparation thereof and the use thereof for the treatment of hypercortisolism and diabetes mellitus or heart insufficiency and myocardial fibrosis.

### BACKGROUND OF THE INVENTION

The adrenal glands of humans are subdivided in two regions, the adrenal medulla and the adrenal cortex. The latter secretes a number of hormones which are known as corticoids and fall into two categories. Glucocorticoids (mainly hydrocortisone and cortisol) act primarily on the carbohydrate and glucose metabolism, and secondarily they can delay wound healing by interfering with the inflammatory events and the formation of fibrous tissue. The second category, the mineral corticoids, are primary participants in the retention of sodium and the excretion of potassium. The most important and effective mineral corticoid is aldosterone.

The biosynthesis of glucocorticoids is controlled, inter alia, by adrenocorticotropin (ACTH). Steroid-11β-hydroxylase (CYP11B1) is the key enzyme of the biosynthesis of glucocorticoids in humans. In all diseases accompanied by increased cortisol formation, this enzyme could thus play a key role. Such clinical pictures include hypercortisolism, especially Cushing's syndrome, and a special form of diabetes mellitus which is characterized by an extreme matutinal rise of the cortisol plasma level.

In the case of Cushing's syndrome, the therapy is usually effected depending on the cause of the disease. A distinction is made between pituitary-hypothalamic or adrenal causes of Cushing's syndrome, the latter developing due to corticoid-producing tumors of the adrenal cortex.

For the therapy of pituitary-hypothalamic Cushing's syndrome, there are usually employed neuromodulatory substances, such as bromocriptine, cyproheptadine, somatostatin or valproic acid, which are supposed to reduce the cortisol production due to their influence on ACTH release. In the past, this therapy was found to be little effective.

In the adrenal Cushing's syndrome, a therapy with inhibitors of steroid biosynthesis is effected especially when a surgical removal of the primary tumor is not possible. What is employed is the non-specific CYP enzyme inhibitors aminoglutethimide, metyrapone, ketoconazole and mitotane, which are frequently applied in the form of a combination therapy. However, in the case of aminoglutethimide, the action on the steroid genesis is based on an attack on CYP11A1, i.e., desmolase, or in the case of ketoconazole, it is based on an inhibition of CYP17. The other compounds mentioned also act non-specifically. Both the combination of several non-selective inhibitors of the steroidogenic CYP enzymes and the high doses that have to be employed are not therapeutically safe. This is important mainly in view of the fact that a lifelong therapy has to be performed which is prone to severe side effects due to the lack of selectivity of the compounds mentioned (Nieman, L. K., Pituitary 5: 77-82 (2002)). One approach is the therapy with highly selective inhibitors of the key enzyme of glucocorticoid synthesis, CYP11B1. Selectivity of the compounds is desired in this case too lest side effects should occur as described in the past, especially on the androgen formation in males (ketoconazole) or on the biosynthesis of mineral corticoids.

Increased cortisol levels are also associated with neurodegenerative diseases. The decrease of the memory and learning capacities upon exposure to increased concentrations of both exogenous and endogenous glucocorticoids (cortisol) has been described (Heffelfinger et al., Dev. Psychopathol. 13: 491-513 (2001)).

In a special form of stress-dependent diabetes mellitus, a rapid matutinal rise of the plasma cortisol level occurs. Further, in diabetes mellitus, increased cortisol levels are associated with the formation of insulin resistance and adverse affection of glucose tolerance (Phillips et al., J. Clin. Endocrinol. Metab. 83: 757-760 (1998)). In this case too, the inhibition of glucocorticoid biosynthesis by a direct and selective inhibition of the key enzyme CYP11B1 could be a therapeutic alternative.

Aldosterone secretion is regulated by a wide variety of signals: the plasma levels of sodium and potassium and the multi-step renin-angiotensin-aldosterone system (RAAS). In this system, the kidneys secrete renin in response to hypotension, and renin releases angiotensin I from a precursor peptide. Angiotensin I in turn is cleaved into angiotensin II, which comprises 8 amino acids and is a potent vasoconstrictor. In addition, it acts as a hormone for the stimulation of aldosterone release (Weber, K. T. & Brilla, C. G., Circulation 83: 1849-1865 (1991)).

The key enzyme of the biosynthesis of mineral corticoids, CYP11B2 (aldosterone synthase), a mitochondrial cytochrome P450 enzyme, catalyzes, the formation of the most potent mineral corticoid, aldosterone, from its steroidal substrate 11-deoxycorticosterone (Kawamoto, T. et al., Proc. Natl. Acad. Sci. USA 89: 1458-1462 (1992)). Increased plasma aldosterone levels are related to clinical pictures such as congestive heart failure and congestive heart insufficiency, myocardial fibrosis, ventricular arrhythmia, stimulation of cardiac fibroblasts, cardiac hypertrophy, renal hypoperfusion and hypertension, and they are involved in the progression of such diseases (Brilla, C. G., Herz 25: 299-306 (2000). Especially in patients suffering from chronic heart insufficiency or renal hypoperfusion or kidney arteriostenoses, the physiological effect of the renin-angiotensin system (RAAS) is replaced by its pathophysiological activation (Young, M., Funder, J. W., Trends Endocrinol. Metab. 11: 224-226 (2000)). Angiotensin-II-mediated vasoconstriction and the water and sodium restriction occurring due to the increased aldosterone levels result in an additional load on the myocardium, which is already primarily insufficient. In a kind of vicious circle, a further reduction of renal perfusion and an increased renin secretion occur. In addition, both the increased plasma aldosterone and angiotensin II levels and aldosterone locally secreted in the heart induce fibrotic structural changes of the myocardium, as a consequence of which the evolution of a myocardial fibrosis leads to a further reduction of the heart performance (Brilla, C. G., Cardiovasc. Res. 47: 1-3 (2000); Lijnen, P. & Petrov, V. J. Mol. Cell. Cardiol. 32: 865-879 (2000)).

Fibrotic structural changes are characterized by the formation of tissue that is characterized by an abnormally high amount of fibrotic material (mainly collagen strands). Such fibroses are beneficial in some situations, such as wound healing, but may be deleterious, for example, when they adversely affect the function of interior organs. In myocardial fibrosis, the heart muscle is traversed by fibrotic strands which render the muscle stiff and inflexible and thereby affect its function.

Since the mortality is 10-20% even for patients with a slight heart insufficiency, it is absolutely necessary to interfere with a suitable medicamentous therapy. In spite of long-term therapies with digitalis glycosides, diuretics, ACE inhibitors or AT-II antagonists, the plasma aldosterone levels remain increased in the patients, and the medication has no effect in terms of the fibrotic structural changes.

Numerous patents and patent applications already relate to mineral corticoid antagonists, especially aldosterone-blocking drugs. Thus, it is known that the steroidal mineral corticoid antagonist spironolactone (17-hydroxy-7-alpha-mercapto-3-oxo-17-α-pregn-4-ene-21-carboxylic acid-γ-lactone acetate; Aldactone®) competitively blocks aldosterone receptors from aldosterone and thus prevents the receptor-mediated aldosterone activity. US 2002/0013303, U.S. Pat. No. 6,150,347 and U.S. Pat. No. 6,608,047 describe the dosing of spironolactone for the therapy or prevention of cardiovascular diseases and myocardial fibrosis while the normal electrolyte and water balances of the patients are retained.

The “Randomized Aldactone Evaluation Study (RALES)” (Pitt, B. et al., New Engl. J. Med. 341: 709-717 (1999)) demonstrated impressively that the administration of the aldosterone receptor antagonist spironolactone (Aldactone®) in addition to a basic therapy with ACE inhibitors and loop diuretics could significantly improve the survival rate of patients with severe heart insufficiencies, since the activity of aldosterone was sufficiently inhibited (Kulbertus, H., Rev. Med. Liege 54: 770-772 (1999)). However, the application of spironolactone was associated with severe side effects, such as gynecomastia, dysmenorrhea and breast pain, which are due to the steroidal structure of the substance and the resulting interactions with further steroid receptors (Pitt, B. et al., New Eng. J. Med. 341: 709-717 (1999); MacFadyen, R. J. et al., Cardiovasc. Res. 35: 30-34 (1997); Soberman, J. E. & Weber, K. T., Curr. Hypertens. Rep. 2: 451-456 (2000)).

Mespirenone (15,16-methylene-17-spirolactone) and its derivatives were considered promising alternatives for spironolactine because they exhibit only a low percentage of the antiandrogenic effect of spironolactone (Losert, W. et al., Drug Res. 36: 1583-1600 (1986); Nickisch, K. et al., J Med Chem 30(8): 1403-1409 (1987); Nickisch, K. et al., J. Med. Chem. 34: 2464-2468 (1991); Agarwal, M. K., Lazar, G., Renal Physiol. Biochem. 14: 217-223 (1991)). Mespirenone blocks the aldosterone biosynthesis as part of a complete inhibition of the biosynthesis of mineral corticoids (Weindel, K. et al., Arzneimittelforschung 41(9):946-949 (1991)). Like spironolactone, mespirenone inhibits the aldosterone biosynthesis, but only at very high concentrations.

WO 01/34132 describes methods for the treatment, prevention or blocking of pathogenic changes due to vascular injuries (restenoses) in mammals by administering an aldosterone antagonist, namely eplerenone (an aldosterone receptor antagonist) or related structures which are in part epoxysteroidal and can all be derived from 20-spiroxanes.

WO 96/40255, US 2002/0123485, US 2003/0220312 and US 2003/0220310 describe therapeutical methods for the treatment of cardiovascular diseases, myocardial fibrosis or cardiac hypertrophy by using a combination therapy of an angiotensin II antagonist and an epoxysteroidal aldosterone receptor antagonist, such as eplerone or epoxymexrenone.

The recently published study EPHESUS (“Eplerenone's Heart Failure Efficacy and Survival Study”, 2003) could support the RALES results. Applied in addition to a basic therapy, the first selective steroidal mineral corticoid receptor antagonist eplerone (Inspra®) clearly reduces the morbidity and mortality in patients with acute myocardial infarction and the occurrence of complications, e.g., drop of the left-ventricular ejection fraction and heart failure (Pitt., B. et al., N. Eng. J. Med. 348: 1390-1382 (2003)).

RALES and EPHESUS clearly demonstrated that aldosterone antagonists represent a therapeutic option which is not to be underestimated. However, their side-effect profile results in a demand for substances which have a different structure and mechanism of action from that of spironolactone. A promising alternative is non-steroidal inhibitors of the biosynthesis of mineral corticoids, because it is better to reduce the pathologically increased aldosterone concentration than just to block the receptors. CYP11B2 as a key enzyme offers itself in this connection as a target for specific inhibitors and has been proposed as such already in previous studies (Hartmann, R. et al., Eur. J. Med. Chem. 38: 363-366 (2003); Ehmer, P. et al., J. Steroid Biochem. Mol. Biol. 81: 173-179 (2002)). Thus, the increased generalized aldosterone release and especially the cardiac aldosterone production can be reduced by a well-aimed inhibition of the biosynthesis, which in turn reduces structural changes of the myocardium.

Selective aldosterone synthase inhibitors could also be a promising class of substances which could promote the healing of the impaired myocardial tissue with reduced scar formation after a myocardial infarction and thus reduce the occurrence of severe complications.

WO 01/76574 describes a medicament which comprises an inhibitor of aldosterone formation or one of its pharmaceutically acceptable salts, optionally in combination with other active substances. WO 01/76574 relates to the use of non-steroidal inhibitors of aldosterone formation that were commercially available at the time, especially the (+) enantiomer of fadrozole, a 4-(5,6,7,8-tetrahydroimidazo[1,5-a]pyridine-5-yl)benzonitrile and its synergistic effect with angiotensin II receptor antagonists.

Anastrozole (Arimidex®) and exemestane (Coromasin®) are further non-steroidal aromatase inhibitors. Their field of application is the treatment of breast cancer by inhibiting aromatase, which converts androstendione and testosterone to estrogen.

The human steroid-11β-hydroxylase CYP11B1 shares above 93% homology with human CYP11B2 (Kawamoto, T. et al., Proc. Natl. Acad. Sci. USA 89: 1458-1462 (1992); Taymans, S. E. et al., J. Clin. Endocrinol. Metab. 83: 1033-1036 (1998)). Despite of the high structural and functional similarity between these two enzymes, strong inhibitors of aldosterone synthesis must not affect the steroid-11β-hydroxylase and therefore must be tested for selectivity. In addition, non-steroidal inhibitors of aldosterone synthase should be preferably applicable as therapeutic agents because less side effects on the endocrine system are to be expected. This has been pointed out in previous studies, as has the fact that the development of selective CYP11B2 inhibitors which do not affect CYP11B1 is rendered more difficult by the high similarity between the two enzymes (Ehmer, P. et al., J. Steroid Biochem. Mol. Biol. 81: 173-179 (2002); Hartmann, R. et al., Eur. J. Med. Chem. 38: 363-366 (2003)).

The inhibitors should also affect other P450 (CYP) enzymes as little as possible. The only active substance known today that affects the corticoid synthesis in humans is the aromatase (estrogen synthase, CYP19) inhibitor fadrozole, which is employed in breast cancer therapy. It can also affect aldosterone and cortisone levels, but only from the administration of ten times the therapeutic dosage (Demers, L. M. et al., J. Clin. Endocrinol. Metabol. 70:1162-1166 (1990)).

For inhibitors of the human aldosterone synthase CYP11B2, a test system for the screening of chemical compounds with Schizosaccharomyces pombe cells that stably express human CYP11B2 and for the subsequent testing of the selectivity with V79MZ cells that stably express either CYP11B2 or CYP11B1 has already been developed (Ehmer, P. et al., J. Steroid Biochem. Mol. Biol. 81: 173-179 (2002)). By means of the S. pombe system, 10 exemplary substances were tested, of which one has been identified by means of the V79MZ system as a potent and selective non-steroidal inhibitor of human CYP11B2 (and potent aromatase inhibitor), and four other substances have been identified as inhibitors that are non-selective but more potent towards CYP11B1 (A: CYP11B2 inhibitor; B-D: non-selective CYP11B1 inhibitors):

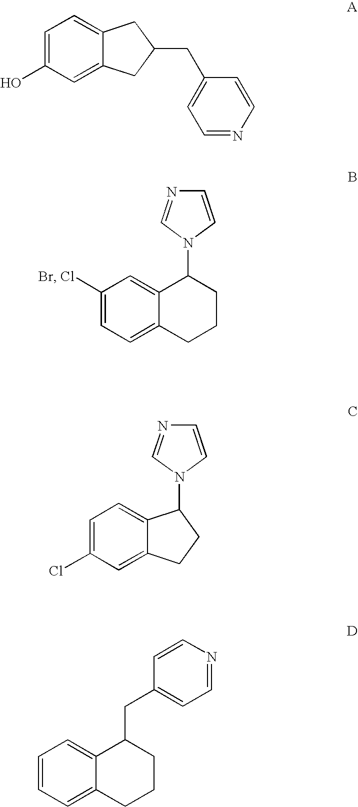

However, this publication was focused on the provision of an effective test system for the screening for selective CYP11B2 inhibitors, and besides the rather general reference to the aromatic N atom and the three structures shown above, it gives only few indications of which classes of substances could be particularly effective ultimately. Further, it may be noted that most structures presented in this publication were strong CYP11B1 inhibitors and therefore should not be candidates for immediate use as selective CYP11B2 inhibitors.

The screening of a P450 inhibitor library of more than 100 substances for inhibitors of bovine aldosterone synthase (CYP18, CYP11B) (in part published in Hartmann, R. W. et al., Arch. Pharm. Pharm. Med. 339, 251-61 (1996)) using the test system presented by Ehmer et al. (Ehmer, P. et al., J. Steroid Biochem. Mol. Biol. 81: 173-179 (2002)) yielded a high number of compounds which had an inhibitory effect on CYP11B2, including the compounds 1a/b and 2a/b (Hartmann, R. et al., Eur. J. Med. Chem. 38: 363-366 (2003)). Within the scope of the cited study, these substances were also tested for their oral availability and further for the in vitro inhibition of human CYP11B2 stably expressed in yeast and, if these tests showed a string inhibition of CYP11B2, in V79MZ cells. Comparisons with the inhibition of other CYPs, including CYP11B1, expressed in V79MZ cells were also performed in order to establish the selectivity of the test substances. By using structural variations, CYP11B2 inhibitors were finally found which showed IC₅₀ values in the low nanomolar range, namely cyclopropatetrahydronaphthalene derivatives and arylmethyl-substituted indanes. It was established that the CYP11B inhibition is strongly influenced by the substituent at the benzene ring and by the heteroaryl radical. The compounds E and F were found as promising leads:

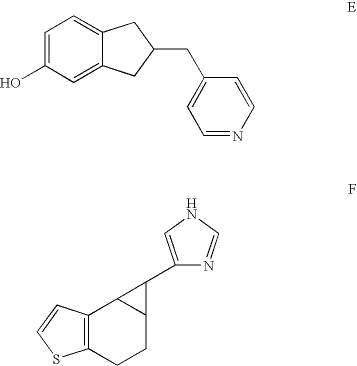

The above scientific publications indicate that the presence of an aromatic nitrogen atom is essential to the complexing of the iron atom in the target enzyme (Ehmer, P. et al., J. Steroid Biochem. Mol. Biol. 81: 173-179 (2002); Hartmann, R. et al., Eur. J. Med. Chem. 38: 363-366 (2003)). In addition, this N atom must be non-substituted and sterically accessible (Ehmer, P. et al., J. Steroid Biochem. Mol. Biol. 81: 173-179 (2002)).

A few heteroarylmethylene-substituted tetrahydronaphthalenes and indanes have been tested for their activities as inhibitors of the non-specific bovine CYP11B already in the run-up to the present invention. However, they proved to be too little specific to be candidates as therapeutic agents for the selective inhibition of CYP11B2 (Mitrenga, M., Dissertation Universität Saarbrücken 1996, Shaker-Verlag, Aachen, Germany (1997)). In addition, the bovine enzyme is not optimally suited for the evaluation of the therapeutic suitability of compounds for the inhibition of human CYPB11 enzymes since the homology between these bovine and human enzymes is not high (75%) (Mornet, E. et. al., J. Biol. Chem. 264: 20961-20967 (1989)).

All inhibitors of aldosterone or glucocorticoid formation known to date have substantial drawbacks: Etomidate and metyrapone inhibit the glucocorticoid formation more strongly as compared to the aldosterone formation. Etomidate is a strong narcotic, and metyrapone is a relatively non-selective CYP inhibitor, which is used only as a diagnostic agent for this reason. For fadrozole, it is described that it inhibits the aldosterone formation more strongly as compared to the glucocorticoid formation (Bhatnagar, A. S. et al., J. Steroid Biochem. Mol. Biol. 37: 1021-1027 (1990); Hausler, A. et al., J. Steroid Biochem. 34: 567-570 (1989); Dowsett, M. et al., Clin. Endocrinol. (Oxf.) 32: 623-634 (1990); Santen, R. J. et al., J. Clin. Endocrinol. Metabol. 73: 99-106 (1991); Demers, L. M. et al., J. Clin. Endocrinol. Metabol. 70: 1162-1166 (1990)). This substance is not a candidate for application as an inhibitor of the aldosterone or glucocorticoid formation either, because it is a very potent aromatase inhibitor and therefore highly interferes with the formation of sexual hormones. In the light of the above prior art, there has been a need for potent and selective inhibitors of the 11′-hydrolase CYP11B1 and the aldosterone synthase CYP 11B2.

On the other hand, 1-heteroarylmethylidene-substituted indanes have been known from the following publications:

From WO 97/12874, there have been known compounds of the following formula (I):

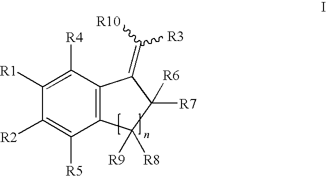

wherein three of R¹, R², R³, R⁵ are independently hydrogen, C₁₋₄-alkyl, C₂₋₄-alkenyl, C₃₋₆-cycloalkyl, hydroxy, C₁₋₄-alkoxy, C₁₋₄-hydroxyalkyl, halogen, nitro or optionally substituted amino, and one of R¹, R², R³, R⁵ is hydrogen; R³ is a 4-imidazolyl radical; R⁶ is hydrogen; R⁷ is hydrogen or C₁₋₄-alkyl; R⁸ is hydrogen, C₁₋₄-alkyl, hydroxy or C₁₋₄-alkoxy; R⁹ is hydrogen or C₁₋₄-alkyl; R¹⁰ is hydrogen or C₁₋₄-alkyl; and n=1 or 2. These compounds have affinity for α2 receptors and are suitable, inter alia, for the treatment of hypertension, glaucoma, chronic or acute pain.

From WO 01/51472, compounds according to the above shown formula (I) have been known wherein R¹, R², R⁴ and R⁵ are hydrogen, or one to three of R¹, R², R⁴, R⁵ are independently halogen, hydroxy, NH₂, halo-C₁₋₆-alkyl, C₁₋₆-alkyl, C₁₋₆-alkoxy or HO—(C₁₋₆)-alkyl; R³ is a 4-imidazolyl radical; R⁶ and R⁷ are hydrogen; one of the radicals R⁶, R⁷, R⁸ and R⁹ is a cyclic radical selected from phenyl, naphthyl, tetrahydronaphthyl, C₃₋₇-cycloalkyl and C₅₋₇-cycloalkenyl, or a methyl radical which bears such a cyclic radical and optionally one or two C₁₋₆-alkyl radicals, two of the radicals R⁶, R⁷, R⁸ and R⁹ are independently hydrogen, hydroxy, C₁₋₆-alkyl, halo-C₁₋₆-alkyl, C₁₋₆-alkoxy or hydroxy-(C₁₋₆)-alkyl; and the remaining radicals R⁶, R⁷, R⁸ and R⁹ are hydrogen; R¹⁰ is hydrogen or C₁₋₆-alkyl; and n=1 or 2. These compounds have affinity for α2 receptors and are suitable for the treatment of diseases of the central nervous system as well as of diseases of the peripheral system, such as diabetes and sexual dysfunction.

From U.S. Pat. No. 3,442,893, compounds according to the above shown formula (I) have been known wherein R¹ is hydrogen, hydroxy, methoxy or alkylcarbonyloxy; R² is hydroxy, alkylcarbonyloxy or methoxy; R³ is a 4-pyridyl or 4-piperidyl radical; R⁴ to R¹⁰ are hydrogen; and n=1. From DE 16 45 952, compounds according to the above shown formula (I) have been known wherein R¹ is hydrogen; R² is hydroxy, C₁₋₄-alkoxy or C₁₋₄-alkylcarbonyloxy; R³ is a 4-pyridyl radical or 4-piperidyl radical; R⁴ to R⁷, R⁸ and R⁹ are hydrogen; R¹⁰ is hydrogen or C₁₋₂-alkyl; and n=2. These compounds are suitable as growth regulators for the gonads of warm-blooded animals.

Kumler, P. L. and Dybas, R. A., J. Org. Chem. 35(11), 3825-3831 (1970), examine the behavior of compounds according to the above shown formula (I) in photocyclization reactions wherein R¹, R², R⁴, R⁵, R⁸ to R¹⁰ are hydrogen; R³ is a 2-pyridyl radical; R⁶ and R⁷ are both hydrogen or both methyl; and n=1 or 2.

Reimann, E. und Hargasser, E., Arch. Pharm. 322, 159-164 (1989), describe the synthesis of compounds according to the above shown formula (I), wherein R¹ is hydrogen; R² is hydrogen or methoxy; R³ is a 3-(4-methyl)pyridyl radical; R⁴ to R¹⁰ are hydrogen; and n=2.

Finally, Vanelle, P. et al., Tetrahedron 47(28), 5173-5184 (1991), describe a compound according to the above shown formula (I), wherein R¹, R², R⁴ to R¹⁰ are all hydrogen; R³ is 3-nitroimidazo[1,2-a]pyrid-2-yl; and n=2.

The synthesis of 1-heteroarylmethylene-substituted indanes by the Pd(PPh₃)₄-catalyzed reaction of 4-(o-iodophenyl)-1-butyne with heteroarylzinc chloride has been described previously (Luo, F. T. & Wang, R. T., Heterocycles 31(8): 1543-1548 (1990)). However, in this scientific publication, only the basic skeleton in Z configuration was presented without further substituents on the C atoms of indane or the heterocycle. Further examples with nitrogen heterocycles are mentioned: Z-2-(1-undanylidenemethyl)pyridine, Z-3-(1-undanylidenemethyl)-pyridine (CAS132819-71-7), Z-2-(1-undanylidenemethyl)-r-methylpyrrole and Z-2-(undanylidenemethyl)benzothiazole.

The synthesis of the E isomers is not possible by this route, and further, expensive chemicals (Pd and Zn derivatives) are required on this synthetic route. A preparation which suitable for a large number of differently substituted tetralines or indanes via the corresponding tetralones or indanones, respectively, has not been known to date.

In the run-up to the present application, a synthesis for the preparation of imidazolyl-substituted indanes was developed (Mitrenga, M., Dissertation Universität Saarbrücken 1996, Shaker-Verlag, Aachen, Germany (1997)); it is based on the following synthetic scheme:

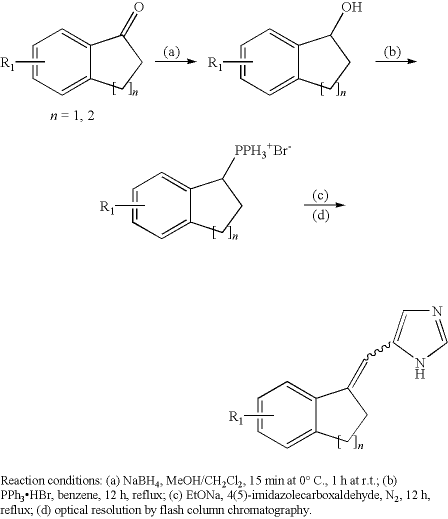

However, in comparative experiments (see Ex. 3), it was found that this reaction is not reproducible for all imidazole derivatives. The difficulty in this reaction evidently resides in the production of the imidazolyl anion by NaOEt. This anion does not seem to be particularly stable. Moreover, the synthesis was only suitable for the preparation of imidazolyl compounds, since the imidazolyl aldehyde employed was employed as a base and at the same time as a reactant.

Therefore, there has been a need for a synthetic process for heteroarylmethylene-substituted indanes and tetralines which is applicable for a broad range of heteroaryls and enables accession to E and Z isomers of the methylidene compounds.

### SUMMARY OF THE INVENTION

It was found that certain aromatic compounds are suitable for the selective inhibition of the 11β-hydroxylase CYP11B1 and/or the aldosterone synthase CYP11B2. Their biological activity with respect to the inhibition of bovine CYP11B and human CYP11B2, CYP11B1, and for establishing the selectivity of human CYP17 (17α-hydroxylase-C17,20-lyase, a key enzyme of the biosynthesis of androgens) and CYP19, was examined. As compared to CYP11B2 inhibitors already described (Hartmann, R. W. et al., Eur. J. Med. Chem. 38: 363-366 (2003)) and to known inhibitors of the corticoid biosynthesis (fadrozole) or steroid biosynthesis (ketoconazole), the compounds presented in the following are more potent and selective.

Further, a suitable synthetic process for these aromatic compounds whose main representatives are imidazolylmethylenetetrahydronaphthalenes and -indanes has been developed.

Thus, the present invention relates to:

(1) the use of a compound having the structure of formula (I)

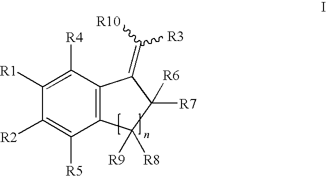

wherein
R¹ and R² are independently selected from H, halogen, CN, hydroxy, nitro, alkyl, alkoxy, alkylcarbonyl, alkylcarbonyloxy, alkylsulfinyl and alkylsulfonyl (the alkyl radicals being straight or branched-chain or cyclic, saturated or unsaturated, and optionally substituted with 1 to 3 radicals R¹²); aryl and heteroaryl radicals and their partially or completely saturated equivalents, optionally substituted with 1 to 3 radicals R¹²; aryloxy- and heteroaryloxy radicals, wherein aryl and heteroaryl have the above meanings, —COOR¹¹, —SO₃R¹¹, —CHO, —CHNR¹¹, —N(R¹¹)₂, —NHCOR¹¹ and —NHS(O)₂R¹¹;
R³ is selected from nitrogen-containing monocyclic or bicyclic heteroaryl radicals and their partially or completely saturated equivalents, optionally substituted with 1 to 3 radicals R¹² and comprising at least one nitrogen atom that is not bound to the methylidene carbon atom and not substituted;
R⁴, R⁵, R⁶, R⁷, R⁸, R⁹ and R¹⁰ are independently selected from H, halogen, CN, hydroxy, nitro, lower alkyl, lower alkoxy, (lower alkyl)carbonyl, (lower alkyl)carbonyloxy, (lower alkyl)carbonylamino, (lower alkyl)sulfonylamino, (lower alkyl)thio, (lower alkyl)sulfinyl and (lower alkyl)sulfonyl (the lower alkyl radicals being straight or branched-chain or cyclic, saturated or unsaturated, and optionally substituted with 1 to 3 radicals R¹²); —N(R¹¹)₂, —COOR¹¹ and —SO₃R¹¹; or
R⁸ or R⁹ together with R⁶ or R⁷ and/or with R⁸ or R⁹ of the neighboring carbon atom form one or two double bonds; or
R⁸ (and R⁹) together with R⁶ (and R⁷) or with R⁸ (and R⁹) of the neighboring carbon atom and the related carbon atoms form a saturated or unsaturated anellated aryl or heteroaryl ring, wherein the atoms of said anellated aryl or heteroaryl ring may be substituted with 1-3 radicals R¹²; or
R⁴ and R¹⁰ together form a methylene, ethylene or ethylidene bridge, wherein the atoms of the bridge may be substituted with one or two radicals R¹²; or
a ring atom in the ortho position of the heteroaryl radical of R³ forms a bond with R⁶ and/or R⁷ directly or through a methylene or methylidene bridge, wherein the bridge atom may be substituted with one or two radicals R¹²;
R¹¹ independently of the occurrence of other R¹¹ radicals is selected from H, lower alkyl (which may be straight or branched-chain or cyclic, saturated or unsaturated, and optionally substituted with 1 to 3 radicals R¹²) and aryl which may be substituted with 1 to 3 radicals R¹²;
R¹² independently of the occurrence of other R¹² radicals is selected from H, hydroxy, —CN, —COOH, —CHO, nitro, amino, mono- and bis-(lower alkyl)amino, lower alkyl, lower alkoxy, (lower alkyl)carbonyl, (lower alkyl)carbonyloxy, (lower alkyl)carbonylamino, (lower alkyl)thio, (lower alkyl)sulfinyl, (lower alkyl)sulfonyl, hydroxy(lower alkyl), hydroxy(lower alkoxy), hydroxy(lower alkyl)carbonyl, hydroxy(lower alkyl)carbonyloxy, hydroxy(lower alkyl)carbonylamino, hydroxy-(lower alkyl)thio, hydroxy(lower alkyl)sulfinyl, hydroxy(lower alkyl)sulfonyl, mono- and bis(hydroxy(lower alkyl)amino and mono- and polyhalogenated (lower alkyl) (wherein the (lower alkyl) radicals may be straight or branched-chain or cyclic, saturated or unsaturated);
n is an integer of from 1 to 3;
or a pharmaceutically acceptable salt thereof
for the treatment of hypercortisolism, diabetes mellitus, heart insufficiency and myocardial fibrosis;
(2) the compound of formula (I) or its pharmaceutically acceptable salts, wherein all the variables have the meaning as stated under (1), with the proviso that:
(a) if n=1, R¹, R² and R⁴-R¹⁰ are hydrogen, then R³ is not 4-imidazolyl or 4-pyridyl;
(b) if n=2, R² and R⁴-R¹⁰ are hydrogen and R¹ is Cl or CN, then R³ is not 4-imidazolyl;
(c) if n=2, R¹ and R⁴-R¹⁰ are hydrogen and R² is CN, then R³ is not 4-imidazolyl;
(d) if n=1, R¹ and R⁴-R¹⁰ are hydrogen and R² is F, Cl, Br or CN, then R³ is not 4-imidazolyl;
(e) if n=2, R¹, R² and R⁴-R¹⁰ are hydrogen, then R³ is not 4-imidazolyl, 4-pyridyl, 4-methyl-3-pyridyl or 3-nitroimidazo[1,2-a]pyrid-2-yl;
(f) if n=1 or 2; three of the radicals R¹, R², R⁴ and R⁵ are independently hydrogen, C₁₋₄-alkyl, C₂₋₄-alkenyl, C₃₋₇-cycloalkyl, hydroxy, C₁₋₄-alkoxy, hydroxy-C₁₋₄-alkyl, halogen, trifluoromethyl, nitro or optionally substituted amino and the fourth radical of R¹, R², R⁴ and R⁵ is hydrogen, R⁶ is hydrogen, R⁷ is hydrogen or C₁₋₄-alkyl, R⁸ is hydrogen, C₁₋₄-alkyl, hydroxy or C₁₋₄-alkoxy, R⁹ and R¹⁰ are independently hydrogen or C₁₋₄-alkyl, then R³ is not 4-imidazolyl;
(g) if n=1 or 2, three of the radicals R¹, R², R⁴ and R⁵ are independently hydrogen, hydroxy, amino, halo-C₁₋₆-alkyl, C₁₋₆-alkyl, C₁₋₆-alkoxy or hydroxy-C₁₋₆-alkyl and the fourth radical of R¹, R², R⁴ and R⁵ is hydrogen, one of the radicals R⁶, R⁷, R⁸ and R⁹ is C₃₋₇-cycloalkyl, C₅₋₇-cycloalkenyl, C₃₋₇-cycloalkylmethyl or C₃₋₇-cycloalkenylmethyl, wherein the methyl radical may be substituted with one or two C₁₋₆-alkyl radicals, two of the radicals R⁶, R⁷, R⁸ and R⁹ are independently hydrogen, hydroxy, C₁₋₆-alkyl, halo-C₁₋₆-alkyl, C₁₋₆-alkoxy or hydroxy-C₁₋₆-alkyl, and the remaining radicals R⁶, R⁷, R⁸ and R⁹ are hydrogen, R¹⁰ is hydrogen or C₁₋₆-alkyl, then R³ is not 4-imidazolyl;
(h) if n=1, R¹ is hydrogen, hydroxy, alkoxy or alkylcarbonyloxy, R² is hydroxy, alkylcarbonyloxy or alkoxy, R⁴-R¹⁰ are hydrogen, then R³ is not 4-pyridyl;
(i) if n=2, R¹ is hydrogen, R² is hydroxy, C₁₋₄-alkoxy or C₁₋₄-alkylcarbonyloxy, R⁴-R⁹ are hydrogen, R¹⁰ is hydrogen or C₁₋₄-alkyl, then R³ is not 4-pyridyl;
(j) if n=1, R¹, R², R⁴, R⁵, R⁸-R¹⁰ are hydrogen, R⁶ and R⁷ are both hydrogen or both methyl, then R³ is not 2-pyridyl;
(k) if n=2, R¹, R², R⁴, R⁵ and R⁸-R¹⁰ are hydrogen, R⁶ and R⁷ are both methyl, then R³ is not 2-pyridyl;
(l) if n=2, R¹ is hydrogen, R² is hydrogen or methoxy, R⁴-R¹⁰ are hydrogen, then R³ is not 4-methyl-3-pyridyl;
or their pharmaceutically acceptable salts;
(3) a process for synthesizing the compounds according to (2), comprising the reduction of compound (II):

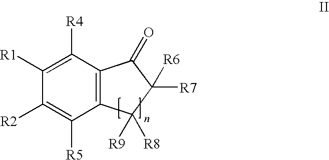

to the corresponding alcohol, followed by a Wittig reaction with compound (III)

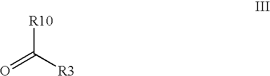

wherein the variables have the meaning as stated under (2), and functional groups in R¹-R¹⁰ may optionally be provided with suitable protective groups;
(4) a pharmaceutical composition containing a compound as defined under (2); and
(5) the use of the compounds as defined under (1) for the selective inhibition of mammal P450 oxygenases, for the inhibition of human or mammal aldosterone synthase or steroid-11β-hydroxylase, especially for the inhibition of human steroid-11β-hydroxylase CYP11B1 or aldosterone synthase CYP11B2, especially for the selective inhibition of CYP11B2 while human CYP11B1 is little affected.

### DETAILED DESCRIPTION OF THE INVENTION

In the compounds of formulas (I), (II) and (III) of the invention, the variables and the expressions used for their characterization have the following meanings:

“Alkyl radicals” and “alkoxy radicals” within the meaning of the invention may be straight or branched-chain or cyclic and saturated or (partially) unsaturated. Preferred alkyl radicals and alkoxy radicals are saturated or have one or more double and/or triple bonds. For straight or branched-chain alkyl radicals, those having from 1 to 10 carbon atoms, especially those having from 1 to 6 carbon atoms, are especially preferred. For the cyclic alkyl radicals, mono- or bicyclic alkyl radicals having from 3 to 15 carbon atoms, especially monocyclic alkyl radicals having from 3 to 8 carbon atoms, are especially preferred.

“Lower alkyl radicals” and “lower alkoxy radicals” within the meaning of the invention are straight or branched-chain or cyclic saturated lower alkyl radicals and lower alkoxy radicals or those having a double or triple bond. For the straight-chain ones, those having from 1 to 6 carbon atoms, especially those having from 1 to 3 carbon atoms, are especially preferred. For the cyclic ones, those having from 3 to 8 carbon atoms are especially preferred.

“Aryls” within the meaning of the present invention comprise mono-, bi- and tricyclic aryl radicals having from 3 to 18 ring atoms which may optionally be anellated with one or more saturated rings. Particularly preferred are anthracenyl, dihydronaphthyl, fluorenyl, hydrindanyl, indanyl, indenyl, naphthyl, naphthenyl, phenanthrenyl, phenyl and tetralinyl.

Unless stated otherwise, “heteroaryl radicals” are mono- or bicyclic heteroaryl radicals having from 3 to 12 ring atoms preferably comprising from 1 to 5 heteroatoms selected from nitrogen, oxygen and sulfur, optionally anellated with one or more saturated rings. The preferred nitrogen-containing monocyclic and bicyclic heteroaryls comprise benzimidazolyl, benzothiazolyl, benzoxazolyl, quinazolinyl, quinolyl, quinoxalinyl, cinnolinyl, dihydroindolyl, dihydroisoindolyl, dihydropyranyl, dithiazolyl, homopiperidinyl, imidazolidinyl, imidazolinyl, imidazolyl, indazolyl, indolyl, isoquinolyl, isoindolyl, isothiazolidinyl, isothiazolyl, isoxazolidinyl, isoxazolyl, morpholinyl, oxadiazolyl, oxazolidinyl, oxazolyl, phthalazinyl, piperazinyl, piperidyl, pteridinyl, purinyl, pyrazolidinyl, pyrazinyl, pyrazolyl, pyrazolinyl, pyridazinyl, pyridyl, pyrimidyl, pyrrolidinyl, pyrrolidin-2-onyl, pyrrolinyl, pyrrolyl, tetrazinyl, tetrazolyl, tetrahydropyrrolyl, thiadiazolyl, thiazinyl, thiazolidinyl, thiazolyl, triazinyl and triazolyl. Particularly preferred are mono- or bicyclic heteroaryl radicals having from 5 to 10 ring atoms preferably comprising from 1 to 3 nitrogen atoms, isoquinolyl, imidazolyl, pyridyl and pyrimidyl being particularly preferred.

“Anellated aryl or heteroaryl rings” within the meaning of the present invention comprise those monocyclic rings with from 5 to 7 ring atoms which are anellated with the neighboring ring through two neighboring ring atoms. They may be saturated or unsaturated. Said anellated heteroaryl rings comprise from 1 to 3 heteroatoms, preferably nitrogen, sulfur or oxygen atoms, more preferably oxygen atoms. Preferred anellated aryl rings are cyclohexyl, cyclohexenyl, cyclopentyl, cyclopentenyl and benzyl, and preferred heteroaryl rings are furanoyl, dihydropyranyl, pyranyl, pyrrolyl, imidazolyl, pyridyl and pyrimidyl.

“Pharmaceutically acceptable salts” within the meaning of the present invention comprise salts of the compounds with organic acids (such as lactic acid, acetic acid, amino acid, oxalic acid etc.), inorganic acids (such as HCl, HBr, phosphoric acid etc.) and, if the compounds have acid substituents, also with organic or inorganic bases. Preferred are salts with oxalic acid and HCl.

Preferred compounds of embodiment (1) of the invention are those of formulas (Ia) to (Ig), the compounds of formulas (Ia), (Ib), (Ic) and (Id) being particularly preferred:

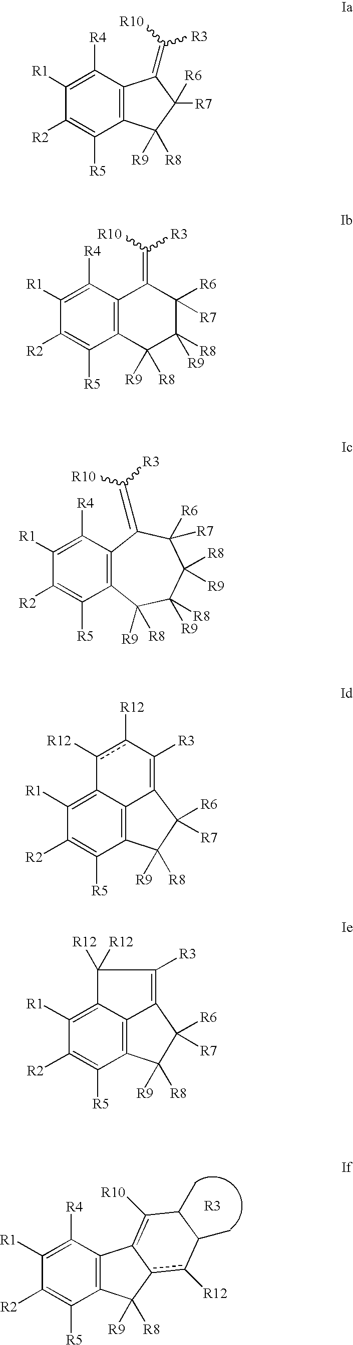

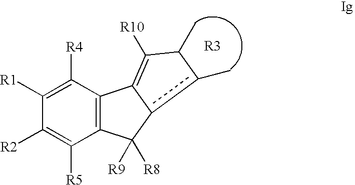

wherein [symbol] is either a single or a double bond, preferably a double bond; and in compound (If) and (Ig), the anellated ring R³ is the residue of the mono- or bicyclic heterocycle R³ as defined above under embodiment (1) of the invention.

A preferred embodiment of compounds (Ia), (Ib) and (Ic) are compounds of the following formula (Ih):

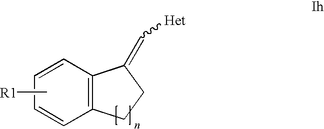

wherein R¹ is H, halogen, CN, O-alkyl, O-alkenyl, O-alkynyl, alkyl, alkenyl or alkynyl, n is 1-3, and Het is a heteroaromatic with 5-10 ring atoms comprising 1-3 nitrogen atoms, and their pharmaceutically acceptable salts.

A particularly preferred embodiment of compounds (Ia), (Ib) and (Ic) are the compounds of the following formula (II):

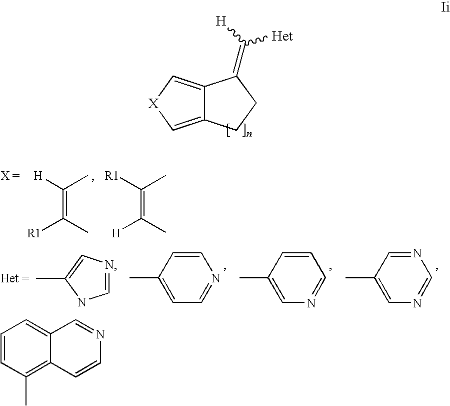

wherein R¹ is H, halogen, CN, O-alkyl, O-alkenyl, O-alkynyl, alkyl, alkenyl or alkynyl, n is 1 or 2, and the double bonds have E or Z configuration, and their pharmaceutically acceptable salts.

Particularly preferred are compounds of formula (I) as defined above under (1) and (2) and those of formulas (Ia) to (Ig) as shown above wherein

(i) R¹ or R² are independently selected from hydrogen, halogen, CN, hydroxy, C₁₋₁₀ alkyl and C₁₋₁₀ alkoxy radicals, wherein said alkyl radicals or alkoxy radicals are straight or branched chain and may be substituted with 1 to 3 radicals R¹²; and/or
(ii) R³ is selected from nitrogen-containing monocyclic heteroaryl radicals with 5-10 ring atoms comprising 1 to 3 nitrogen atoms, especially selected from isoquinolyl, imidazolyl, oxazolyl, pyrazinyl, pyrazolyl, pyridyl, pyrimidyl, pyrrolyl, thiazolyl, triazinyl and triazoyl; and/or
(iii) R⁴, R⁵, R⁶, R⁷, R⁸, R⁹, R¹⁰ are independently selected from H, halogen, CN, hydroxy and C₁₋₆ alkyl and C₁₋₆ alkoxy radicals which may be substituted with 1 to 3 radicals R¹²; and/or
(iv) R¹² is selected from H, halogen, hydroxy, CN, C₁₋₃-alkyl and C₁₋₃-alkoxy; and/or
(v) n is 1 or 2.

Of those, especially preferred are those compounds of formulas (I) and (Ia) to (Ig), especially compounds for formulas (Ia) to (Ic), in which

(i) R¹ or R² is hydrogen;
(ii) the other of substituents R¹ or R² is selected from H, fluorine, chlorine, CN, hydroxy, C₁₋₃-alkyl and C₁₋₃-alkoxy;
(iii) R³ is selected from isoquinolyl, pyridyl, imidazolyl and pyrimidyl; and

### (iv) R⁴, R⁵, R⁶, R⁷, R⁸, R⁹, R¹⁰ are H.

Of those, further preferred are compounds of formula (Id) in which

(i) R¹ or R² is hydrogen;
(ii) the other of substituents R¹ or R² is selected from H, fluorine, chlorine, CN, hydroxy, C₁₋₃-alkyl and C₁₋₃-alkoxy;
(iii) R³ is selected from pyridyl, imidazolyl, isoquinolyl and pyrimidyl;

### (iv) R⁴, R¹, R⁶, R⁷, R⁸, R⁹ and R¹² are H; and

(v) [symbol] is a double bond.

Depending on the position of substituents R³ and R¹⁰, the compounds of formula (I) may be in E or Z configuration. The present invention includes both the mixture of isomers and the isolated E and Z compounds. Also, the compounds (I) have centers of chirality (e.g., the carbon atoms substituted with R⁶/R⁷ and R⁸/R⁹). In this case too, both the mixtures of stereoisomers and the isolated individual compounds are included in the invention.

Preferred compounds of formula (I) are the following compounds:
E,Z-3-(1,2,3,4-tetrahydronaphth-1-ylidenemethyl)-pyridine,
E,Z-3-(6-fluoro-1,2,3,4-tetrahydronaphth-1-ylidenemethyl)-pyridine,
E,Z-3-(6-chloro-1,2,3,4-tetrahydronaphth-1-ylidenemethyl)-pyridine,
E,Z-3-(6-methoxy-1,2,3,4-tetrahydronaphth-1-ylidenemethyl)-pyridine,
E,Z-3-(7-fluoro-1,2,3,4-tetrahydronaphth-1-ylidenemethyl)-pyridine,
E,Z-3-(7-chloro-1,2,3,4-tetrahydronaphth-1-ylidenemethyl)-pyridine,
E,Z-3-(7-methoxy-1,2,3,4-tetrahydronaphth-1-ylidenemethyl)-pyridine,
E,Z-4-(1,2,3,4-tetrahydronaphth-1-ylidenemethyl)-pyridine,
E,Z-4-(6-fluoro-1,2,3,4-tetrahydronaphth-1-ylidenemethyl)-pyridine,
E,Z-4-(6-chloro-1,2,3,4-tetrahydronaphth-1-ylidenemethyl)-pyridine,
E,Z-4-(6-methoxy-1,2,3,4-tetrahydronaphth-1-ylidenemethyl)-pyridine,
E,Z-4-(7-fluoro-1,2,3,4-tetrahydronaphth-1-ylidenemethyl)-pyridine,
E,Z-4-(7-chloro-1,2,3,4-tetrahydronaphth-1-ylidenemethyl)-pyridine,
E,Z-4-(7-methoxy-1,2,3,4-tetrahydronaphth-1-ylidenemethyl)-pyridine,
E,Z-4-(1,2,3,4-tetrahydronaphth-1-ylidenemethyl)-imidazole,
E,Z-4-(6-fluoro-1,2,3,4-tetrahydronaphth-1-ylidenemethyl)-imidazole,
E,Z-4-(6-chloro-1,2,3,4-tetrahydronaphth-1-ylidenemethyl)-imidazole,
E,Z-4-(6-methoxy-1,2,3,4-tetrahydronaphth-1-ylidenemethyl)-imidazole,
E,Z-4-(6-cyano-1,2,3,4-tetrahydronaphth-1-ylidenemethyl)-imidazole,
E,Z-4-(7-fluoro-1,2,3,4-tetrahydronaphth-1-ylidenemethyl)-imidazole,
E,Z-4-(7-chloro-1,2,3,4-tetrahydronaphth-1-ylidenemethyl)-imidazole,
E,Z-4-(7-methoxy-1,2,3,4-tetrahydronaphth-1-ylidenemethyl)-imidazole,
E,Z-3-(1-indanylidenemethyl)-pyridine,
E,Z-3-(5-fluoro-1-indanylidenemethyl)-pyridine,
E,Z-3-(5-chloro-1-indanylidenemethyl)-pyridine,
E,Z-3-(5-bromo-1-indanylidenemethyl)-pyridine,
E,Z-3-(5-methoxy-1-indanylidenemethyl)-pyridine,
E,Z-3-(5-ethoxy-1-indanylidenemethyl)-pyridine,
E,Z-3-(6-fluoro-1-indanylidenemethyl)-pyridine,
E,Z-3-(6-chloro-1-indanylidenemethyl)-pyridine,
E,Z-3-(4-methyl-1-indanylidenemethyl)-pyridine,
E,Z-3-(4-fluoro-1-indanylidenemethyl)-pyridine,
E,Z-3-(4-chloro-1-indanylidenemethyl)-pyridine,
E,Z-3-(7-methoxy-1-indanylidenemethyl)-pyridine,
E,Z-4-(1-indanylidenemethyl)-pyridine,
E,Z-4-(5-fluoro-1-indanylidenemethyl)-pyridine,
E,Z-4-(5-chloro-1-indanylidenemethyl)-pyridine,
E,Z-4-(6-fluoro-1-indanylidenemethyl)-pyridine,
E,Z-4-(6-chloro-1-indanylidenemethyl)-pyridine,
E,Z-5-(1-indanylidenemethyl)-pyrimidine,
E,Z-5-(5-fluoro-1-indanylidenemethyl)-pyrimidine,
E,Z-5-(5-chloro-1-indanylidenemethyl)-pyrimidine,
E,Z-5-(5-methoxy-1-indanylidenemethyl)-pyrimidine,
E,Z-5-(6-fluoro-1-indanylidenemethyl)-pyrimidine,
E,Z-5-(6-chloro-1-indanylidenemethyl)-pyrimidine,
E,Z-5-(6-methoxy-1-indanylidenemethyl)-pyrimidine,
E,Z-4-(1-indanylidenemethyl)-imidazole,
E,Z-4-(5-fluoro-1-indanylidenemethyl)-imidazole,
E,Z-4-(5-chloro-1-indanylidenemethyl)-imidazole,
E,Z-4-(5-bromo-1-indanylidenemethyl)-imidazole,
E,Z-4-(5-methoxy-1-indanylidenemethyl)-imidazole,
E,Z-4-(5-cyano-1-indanylidenemethyl)-imidazole,
E,Z-4-(6-fluoro-1-indanylidenemethyl)-imidazole,
E,Z-4-(6-chloro-1-indanylidenemethyl)-imidazole,
E,Z-4-(6-bromo-1-indanylidenemethyl)-imidazole,
E,Z-4-(6-methoxy-1-indanylidenemethyl)-imidazole,
E,Z-4-(6-cyano-1-indanylidenemethyl)-imidazole and 3-(1,2-dihydroacenaphthylen-3-yl)pyridine.

Of those, especially preferred are:
E,Z-4-(5-chloro-1-indanylidenemethyl)-imidazole,
E,Z-4-(5-fluoro-1-indanylidenemethyl)-imidazole,
E,Z-4-(1,2,3,4-tetrahydronaphth-1-ylidenemethyl)-imidazole,
E,Z-4-(6-cyano-1,2,3,4-tetrahydronaphth-1-ylidenemethyl)-imidazole,
E,Z-4-(7-fluoro-1,2,3,4-tetrahydronaphth-1-ylidenemethyl)-imidazole,
E,Z-4-(7-chloro-1,2,3,4-tetrahydronaphth-1-ylidenemethyl)-imidazole,
E,Z-3-(1-indanylidenemethyl)-pyridine,
E,Z-3-(5-fluoro-1-indanylidenemethyl)-pyridine,
E,Z-3-(5-chloro-1-indanylidenemethyl)-pyridine,
E,Z-3-(4-fluoro-1-indanylidenemethyl)-pyridine,
E,Z-3-(4-chloro-1-indanylidenemethyl)-pyridine,
E,Z-3-(5-methoxy-1-indanylidenemethyl)-pyridine,
E,Z-3-(7-methoxy-1-indanylidenemethyl)-pyridine,
E,Z-3-(5-fluoro-1-indanylidenemethyl)-pyrimidine and
3-(1,2-dihydroacenaphthylen-3-yl)pyridine;
   and especially
Z-4-(5-chloro-1-Indanylidenemethyl)-imidazole,
Z-4-(1,2,3,4-tetrahydronaphth-1-ylidenemethyl)-imidazole,
Z-4-(6-cyano-1,2,3,4-tetrahydronaphth-1-ylidenemethyl)-imidazole,
E-3-(1-indanylidenemethyl)-pyridine,
E-3-(5-fluoro-1-indanylidenemethyl)-pyridine,
E-3-(5-chloro-1-indanylidenemethyl)-pyridine,
E-3-(5-methoxy-1-indanylidenemethyl)-pyridine,
E-3-(4-fluoro-1-indanylidenemethyl)-pyridine,
E-3-(7-methoxy-1-indanylidenemethyl)-pyridine,
E-3-(5-fluoroindanylidenemethyl)-pyrimidine and
3-(1,2-dihydroacenaphthylen-3-yl)pyridine.

In the process according to embodiment (3), the chemical compounds according to the invention can be synthesized by reducing compound (II) to the corresponding alcohol, followed by a Wittig reaction (cf. Ex. 1-3). The process is preferably effected according to the following general synthetic scheme:

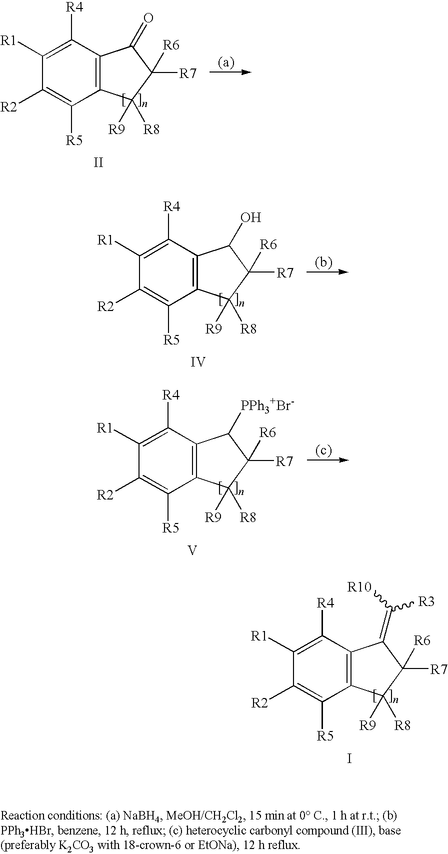

The crucial step of the synthesis is a Wittig reaction using different heterocyclic carbonyl compounds, preferably aldehydes, and suitable salts, especially phosphonium salts, of the bicyclic component. Starting from the corresponding ketones II which are reduced to the corresponding alcohols with a suitable reductant, preferably NaBH₄, alcohol intermediates are formed. These are converted to their phosphonium salts. This is followed by a modified Wittig reaction with a phosphonium salt and heterocyclic carbonyl compound as reactants, a suitable base, especially K₂CO₃, e.g., in dry CH₂Cl₂, and a suitable phase-transfer catalyst, preferably 18-crown-6. For the preparation of imidazole compounds, NaOEt, e.g., in ethanol, without adding a phase-transfer catalyst is preferred as the suitable base.

The mixture of E and Z isomers obtained according to the Wittig reaction can be employed as a mixture or separated into its isomers. The separation is effected by crystallization or chromatographic methods, preferably by column chromatography or flash chromatography. The isomers can be converted to stable salts thereof, preferably to HCl or oxalic acid salts. For the spectroscopic analysis, these are preferably stable hydrochlorides or oxalates, and for the use according to embodiment (1) according to the invention, these are preferably pharmaceutically acceptable salts.

The synthesis according to the invention can be used for the preparation of the E and Z isomers of the compounds according to the invention. By the synthesis according to the invention, the yields can be increased, in part drastically (up to 90%), as compared to previously known methods, in particular, the proportion of Z isomer in the product can be enhanced clearly. Therefore, a preferred application of the synthesis is the preparation of the Z isomers of the compounds according to embodiment (3) of the invention.

In synthesis (3) for the preparation of compounds having substituents or functional groups of the heterocyclic carbonyl compounds that can be deprotonated under the conditions of a Wittig reaction, it is required to provide them with suitable protective groups. Suitable protective groups and their deprotection are available to the skilled person from, e.g., T. W. Green, Protective Groups in Organic Synthesis, Harvard University, John Wiley & Sons (1981). Of course, this means that a downstream deprotection step is necessary when such protective groups are used in process (3) according to the invention. Thus, for example, the synthesis of imidazole derivatives is effected according to the reaction scheme:

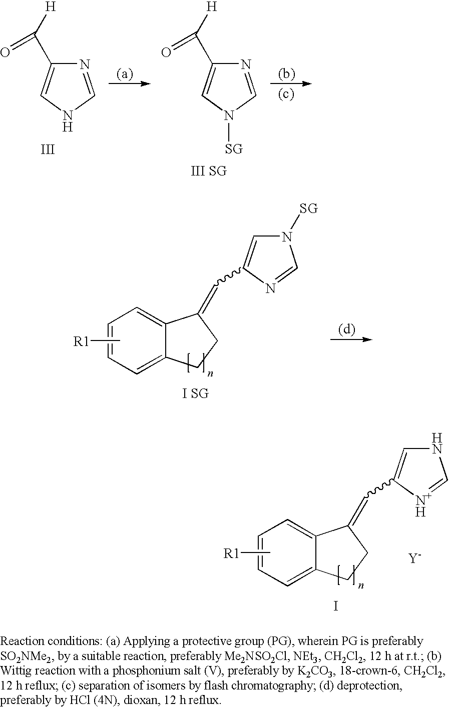

(cf. Example 3, “Alternative Synthesis”). This synthetic process enables the preparation of special imidazole derivatives and their hydrochlorides that, by previously known methods (Mitrenga, M., Dissertation Universität Saarbrücken 1996, Shaker-Verlag, Aachen, Germany (1997)), have been accessible only with a high expenditure or not at all. Preferably, protective groups are applied to the imidazole that remain at the ring during the separation of isomers and thereby clearly facilitate, in particular, the chromatographic separation, which is often difficult for free imidazole groups. These protective groups can be cleaved off by suitable reagents to obtain the final product.

In a four-step synthesis, the compounds of structure (Id) can be prepared from acenaphthene or a suitable derivative thereof, optionally followed by a purification, e.g., by chromatographic separation (see also the following reaction scheme): The nitration of acenaphthene (Chen, M. et al., Ranliao Gongye 38: 21-23 (2001)) and subsequent hydrogenation (Friedman, O. M. et al., J. Am. Chem. Soc. 71: 3010-3013 (1949)) yields, inter alia, 3-aminoacenaphthene. After the following Sandmeyer reaction, the mixture of bromine compounds obtained is isolated and directly reacted with 3-pyridineboronic acid in a Suzuki coupling to yield the desired product. The desired product, the acenaphthene derivative 50, is subsequently isolated, for example, by means of flash column chromatography. The thus prepared oil can be converted to the corresponding hydrochloride to increase its stability.

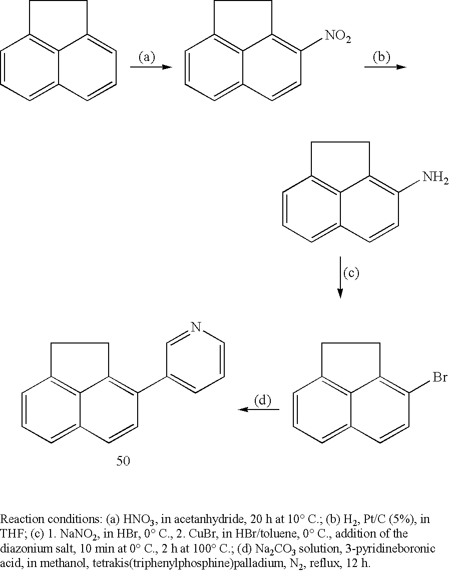

The testing of the compounds according to the invention for usefulness according to embodiment (1) is effected with in vitro test systems, preferably with more than one in vitro test system. The first step of these tests according to the invention includes the testing with non-specific bovine adrenal CYP11B from mitochondria for activity of the test substances (Hartmann, R. et al., J. Med. Chem. 38: 2103-2111 (1995)). The second step includes the testing with human CYP11B enzymes, preferably human CYP11B1 and CYP11B2. These human enzymes can be either expressed recombinantly, especially in Schizosaccharomyces pombe or V79 cells, or in a tested human cell line, especially the adrenocortical tumor cell line NCI-H295R (cf. Ex. 5). For the use of (1) according to the invention, it is particularly preferred to employ substances which show an effect on human CYP11B enzymes, because there is no or only a little correlation between test data with bovine and human enzymes (cf. Ex. 9). For identifying novel therapeutically active compounds according to embodiment (1) for humans, fission yeast and V79MZh cells that recombinantly express CYP11B and CYP11B2 are particularly suitable.

Of the imidazole derivatives, especially suitable for the inhibition of bovine CYP11B according to the invention are the compounds 41b, 42b, 44b, 45a, 45b, 48b, 49b, which show a high percent inhibitory effect on the order of 90% as compared to the non-selective CYP inhibitor ketoconazole (78%) (Table 4).

Of the imidazole derivatives according to embodiment (1) and (2), the Z isomers are particularly suitable for use according to (5), Z-4-(5-chloro-1-indanylidenemethyl)-imidazole 48b being particularly preferred (Ex. 9); the latter is a highly potent CYP11B2 inhibitor (IC₅₀: 4 nM), which has five times the selectivity of CYP11B1 (IC₅₀: 20 nM).

To determine the inhibition of human CYP11B2 by the test compounds, a screening test in recombinant S. pombe, especially S. pombe P1 expressing CYP11B2, can be used (Ex. 5A). Thereafter, for a further examination for the usefulness according to use (5), those compounds are selected, in particular, which show a higher inhibitory effect than the reference fadrozole.

Surprisingly, there is a low correlation or none at all between inhibition values of the bovine and human enzymes.

In a third step, compounds can be tested for usefulness according to (5) in V79 MZh cells (hamster lung fibroblasts) which express either CYP11B1 or CYP11B2 for their activity and selectivity (Ex. 5B). Different inhibition profiles are found: inhibitors which are selective for either CYP11B1 or CYP11B2, and inhibitors able to inhibit both CYP11B enzymes.

For the selective inhibition of CYP11B1 according to embodiment (1) and (2), the imidazole derivatives 41b, 42b, 44b, 45a, 45b, 46a, 48a and 49a, in particular, and the compounds 6a, 8a, 10a, 13b are suitable, and for the selective inhibition of CYP11B2, the imidazole derivatives 48b und 49b as well as many more of the compounds presented herein are suitable (cf. Ex. 6-9). The acenaphthene derivative 50 (“hybrid inhibitor”) was also found to be highly active towards CYP11B2 (IC₅₀=10 nM), and with an IC₅₀=2452 nM, additionally selective towards CYP11B1.

The inhibition of CYP19 by the test compounds can be performed in vitro using human placental microsomes and [1β,2β-³H]testosterone as a substrate (modified according to: Thompson, E. A. Jr. & Siterii, P. K., J. Biol. Chem. 249: 5364-5372 (1974)) (Ex. 4). The chlorine derivative 48b, the strongest CYP11B2 inhibitor, was a weak inhibitor of CYP19 (IC₅₀: 39 nM).

The inhibition of CYP17 by the test substances can be determined in vitro with microsomes from E. coli which recombinantly expresses CYP17 and progesterone as the substrate (Ex. 4). Almost all compounds tested showed no or only a weak inhibition as compared to the reference ketoconazole.

The NCI-H295R cell line is commercially available and is frequently used as a model for the human adrenal cortex. The cells were isolated for the first time in 1980 (Gazdar, A. F. et al., Cancer Res. 50: 5488-5496 (1990)) and contain 5 steroidogenic CYP450 enzymes, including 17-alpha-hydroxylase, CYP11B1 and CYP11B2. Since all the steroidogenic CYP enzymes that occur in the adrenal cortex are expressed in this cell line, it is an important tool in the estimation of the selectivity of inhibitors in vitro. Consequently, an essential difference from the V79 cells is not only the fact that NCI-H295R are human cells, but also the fact that in V79MZh11B1 and V79MZh11B2, only one target enzyme each is recombinantly expressed in a system that is otherwise completely free of CYP enzyme, while NCI-H295R is a substantially more complex model. By using this novel model, the prediction of effects and side effects of compounds on the complex enzymes of the adrenal cortex can become clearly more precise.

The influence of the substances found in the present invention on human CYP11B1 and CYP11B2 in NCI-H295R cells was first tested in an exemplary manner by using only a few compounds (Ex. 10).

In first experiments with a rat model, the in vivo activity of the compounds presented here could be demonstrated. Fadrozole lowers the aldosterone and corticosterone levels in ACTH-stimulated rats (Hausler et al., J. Steroid Biochem. 34: 567-570 (1989)). Some of the compounds presented here, such as 1a, 5a und 48a, in vivo showed a similar behavior to that of fadrozole.

Compound 50 was tested in V79 cells for inhibition of CYP11B1 and CYP11B2. Compound 50 inhibits the human aldosterone synthase in the low nanomolar range and additionally exhibits only a very weak inhibition of human CYP11B1. The substance is not only highly potent, but also very selective.

The substances of formula (I) which are suitable for use according to embodiment (5) can serve for the development of a medicament which improves the life quality of patients suffering from heart insufficiency or myocardial fibrosis and can significantly reduce the mortality. The results of the present invention clearly show that it is possible to develop inhibitors for the target enzyme CYP11B2 which are highly active, but which have a low influence on CYP11B1, which shares a great structural and functional homology with CYP11B2, and vice versa.

Further, the substances of formula (I) which are suitable for use according to embodiment (5) can serve for the development of a medicament which improves the life quality of patients suffering from hypercortisolism or diabetes mellitus and can significantly reduce the mortality. The results of the present invention clearly show that it is possible to develop inhibitors for the target enzyme CYP11B2 which are highly active, but which have a low influence on CYP11B1, which shares a great structural and functional homology with CYP11B2.

The compounds according to the invention are suitable in vitro and in vivo as individual compounds and in combination with other active substances and auxiliary agents, for example, for the inhibition of human and mammal P450 oxygenases, especially for the inhibition of human or mammal aldosterone synthase, more especially for the inhibition of human aldosterone synthase CYP11B2 while human CYP11B1 is little affected, and conversely for the inhibition of CYP11B1 while human CYP11B2 is little affected. The compounds selective for CYP11B2 can be employed for the preparation of medicaments for the therapy of heart insufficiency, myocardial (cardiac) fibrosis, (congestive) heart failure, hypertension and primary hyperaldosteronism in humans and mammals. The compounds selective for CYP11B1 can be employed for the preparation of medicaments for the therapy of hypercortisolism and diabetes mellitus.

These medicaments or the pharmaceutical compositions according to embodiment (4) of the invention may contain other active substances as well as appropriate auxiliary agents and carriers in addition to the compounds according to the invention. Appropriate auxiliary agents and carriers are determined by the skilled person as a function of the field of application and dosage form.

In addition, the invention includes a process and the use of the compound according to the invention for the prevention, deceleration of the progress or therapy of one of the following diseases or clinical pictures: diabetes mellitus, hypercortisolism, hypertension, congestive heart failure, kidney failure, especially chronic kidney failure, restenosis, atherosclerosis, nephropathy, coronary heart diseases, increased formation of collagen, fibrosis, respectively associated or not with occurrence of hypertension, by administering a pharmaceutical formulation according to the invention.

In a preferred embodiment, this process is suitable for the prevention, deceleration of the progress or therapy of myocardial fibrosis, congestive heart failure or congestive heart insufficiency and comprises the administration of an effective dose of an aldosterone synthase inhibitor according to the invention or a pharmaceutically acceptable salt thereof to the afflicted human or mammal.

In a further preferred embodiment, this process is suitable for the prevention, deceleration of the progress or therapy of stress-dependent therapy-resistant diabetes mellitus or hypercortisolism and comprises the administration of an effective dose of a steroid hydroxylase inhibitor according to the invention, especially steroid-11β-hydroxylase inhibitor, or a pharmaceutically acceptable salt thereof to the afflicted human or mammal.

The preferred route of administration for the above mentioned processes is oral application, wherein the content of active substance is to be adapted by the skilled person to the respective therapy and patient.

The invention is further illustrated by means of the following Examples which do not limit, however, the process according to the invention.

### EXAMPLES

Material and analytical methods: Melting point measurements were performed on a Mettler FP1 or Stuart Scientific SMP.3 melting point apparatus and are uncorrected. IR spectra from powders were recorded on a Bruker Vector 33 FT-infrared spectrometer or as KBr or NaCl pellets on a Perkin-Elmer 398-infrared spectrometer. ¹H NMR spectra were recorded on a Bruker AW-80 (80 MHz), AM-400 (400 MHz) or DRX-500 (500 MHz) instrument. Chemical shifts are stated in parts per million (ppm), TMS being the internal standard for recordings in DMSO-d₆ and CDCl₃. All coupling constants (J) are stated in Hz. Elemental analyses were performed at the Lehrstuhl für Anorganische Chemie, Universitat des Saarlandes, Germany. The reagents and solvents are derived from commercial sources and were used without further purification. Flash column chromatography (FCC) was performed over silica gel 60 (40-63 μm), the course of the reaction was monitored by means of thin-layer chromatography over ALUGRAM SIL G/UV₂₅₄ plates (Macherey-Nagel, Düren, Germany).

### Example 1

### Synthesis of the Compounds 1 to 38

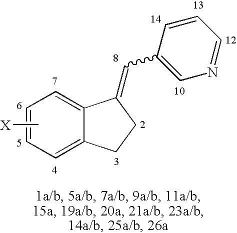

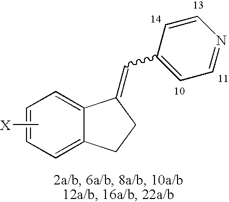

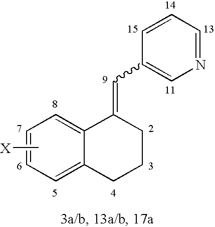

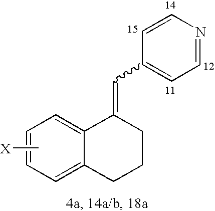

| No. | X | Isomer |
|---|---|---|
| 1a | H | E |
| 1b | H | Z |
| 2a | H | E |
| 2b | H | Z |
| 3a | H | E |
| 3b | H | Z |
| 4a | H | E |
| 5a | 5-F | E |
| 5b | 5-F | Z |
| 6a | 5-F | E |
| 6b | 5-F | Z |
| 7a | 5-Cl | E |
| 7b | 5-Cl | Z |
| 8a | 5-Cl | E |
| 8b | 5-Cl | Z |
| 9a | 5-Br | E |
| 9b | 5-Br | Z |
| 10a | 5-Br | E |
| 10b | 5-Br | Z |
| 11a | 5-OCH₃ | E |
| 11b | 5-OCH₃ | Z |
| 12a | 5-OCH₃ | E |
| 12b | 5-OCH₃ | Z |
| 13a | 6-OCH₃ | E |
| 13b | 6-OCH₃ | Z |
| 14a | 6-OCH₃ | E |
| 14b | 6-OCH₃ | Z |
| 15a | 6-OCH₃ | E |
| 16a | 6-OCH₃ | E |
| 16b | 6-OCH₃ | Z |
| 17a | 6,7-(OCH₃)₂ | E |
| 18a | 6,7-(OCH₃)₂ | E |
| 19a | 5-OEt | E |
| 19b | 5-OEt | Z |
| 20a | 5-OBn | E |
| 21a | 6-CH₃ | E |
| 21b | 6-CH₃ | Z |
| 22a | 6-CH₃ | E |
| 22b | 6-CH₃ | Z |
| 23a | 4-CH₃ | E |
| 23b | 4-CH₃ | Z |
| 24a | 4-F | E |
| 24b | 4-F | Z |
| 25a | 4-Cl | E |
| 25b | 4-Cl | Z |
| 26a | 7-OMe | E |

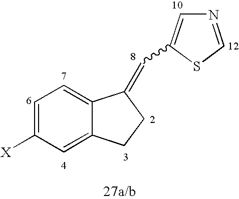

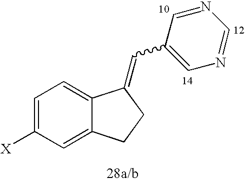

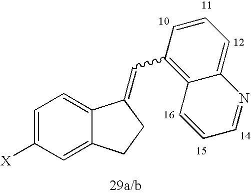

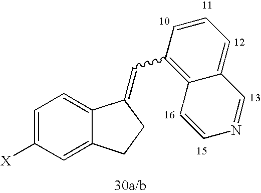

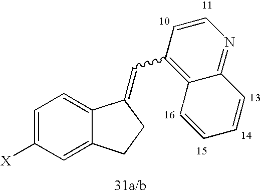

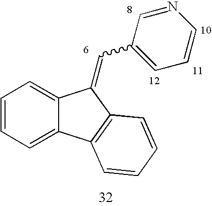

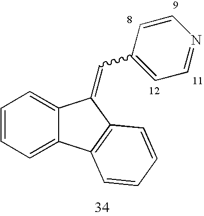

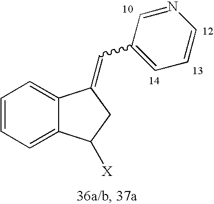

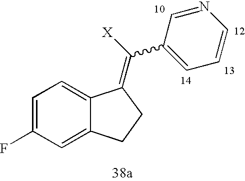

| No. | X | Isomer |
|---|---|---|
| 27a | 5-OMe | E |
| 27b | 5-OMe | Z |
| 28a | 5-F | E |
| 28b | 5-F | Z |
| 29a | 5-F | E |
| 29b | 5-F | Z |
| 30a | 5-F | E |
| 30b | 5-F | Z |
| 31a | 5-F | E |
| 31b | 5-F | Z |
| 32 | — | — |
| 34 | — | — |
| 36a | 3-CH₃ | E |
| 36b | 3-CH₃ | Z |
| 37a | 3-Phenyl | E |
| 38a | CH₃ | E |

The synthesis was effected according to the general synthetic scheme:

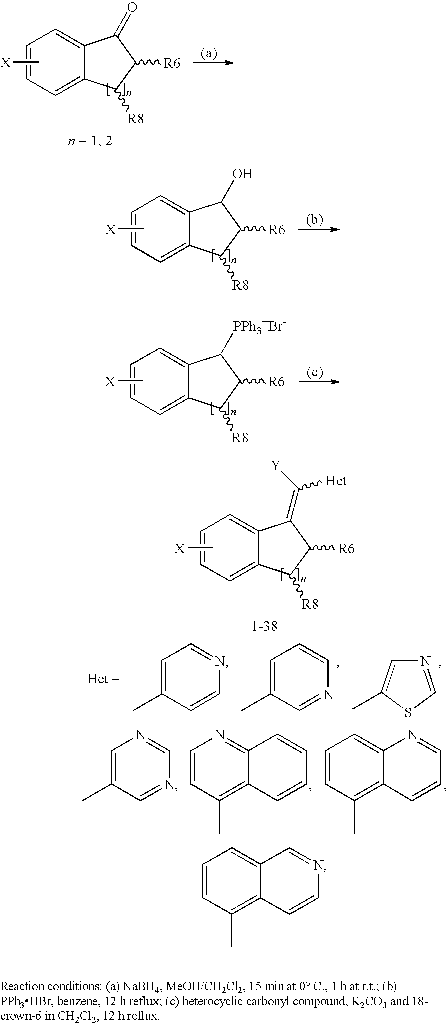

The crucial step of the synthesis was a Wittig reaction using different heterocyclic aldehydes and phosphonium salts of the bicyclic component. Starting from the corresponding ketones which were reduced to the corresponding alcohol with NaBH₄, the indanol and tetrahydronaphthol intermediates were converted to their phosphonium salts using PPh₃.HBr in benzene. This was followed by a modified Wittig reaction with a phosphonium salt and heterocyclic aldehyde as reactants, K₂CO₃ as the base in dry CH₂Cl₂, and a few mg of 18-crown-6 as a phase-transfer catalyst.

The mixture of E and Z isomers obtained according to the Wittig reaction was separated by flash column chromatography. The isomers were converted to stable hydrochlorides or oxalates and characterized by NMR.

A) Synthesis of the commercially unavailable precursors: The following compounds were prepared by known synthetic methods:

### 5-Ethoxyindane-1-one (19i) and 5-(benzyloxy)indane-1-one (20i)

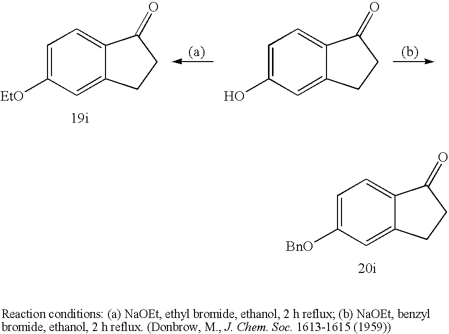

### 6-Methylindane-1-one (21i) and 7-Methoxyindane-1-one (26i)

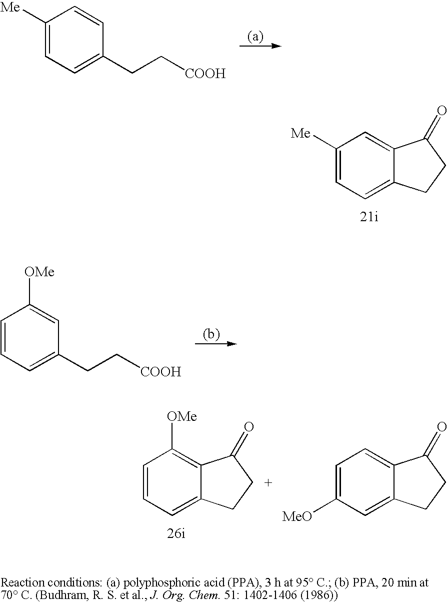

For preparing the 4-substituted indanones, 3-(2-fluorophenyl)propanoic acid (Houghton, R. P. et al., J. Chem. Soc. Perkin. Trans. 1 5: 925-931 (1984)) was synthesized in two steps as the starting material: Knoevenagel reaction of malonic acid with 2-fluorobenzaldehyde (Rabjohn, M., Org. Synth. Collective 327-329 (1963)), followed by a catalytic reduction of the 3-(2-fluorophenyl)-acrylic acid produced (24iv) (Luo, J. K. et al., J. Heterocycl. Chem. 27: 2047-2052 (1990)) with PtO₂.H₂O (Musso, D. L. et al., J. Med. Chem. 46: 399-408 (2003)). The 3-(2-fluorophenyl)propanoic acid (24iii) and the commercially available 3-(2-chlorophenyl)propanoic acid were converted to the acid chlorides 3-(2-fluorophenyl)propanoyl chloride (24ii) and 3-(2-chlorophenyl)propanoyl chloride (25ii) and finally cyclized with AlCl₃ (Musso, D. L. et al., J. Med. Chem. 46: 399-408 (2003)) to form 4-fluoroindane-1-one (24i) (Olivier, M. & Marechal, E., Bull. Soc. Chim. Fr., 3092-3095 (1973)) and 4-chloroindane-1-one (25i) (Takeuchi, R. & Yasue, H., J. Org. Chem. 58: 5386-5392 (1993)):

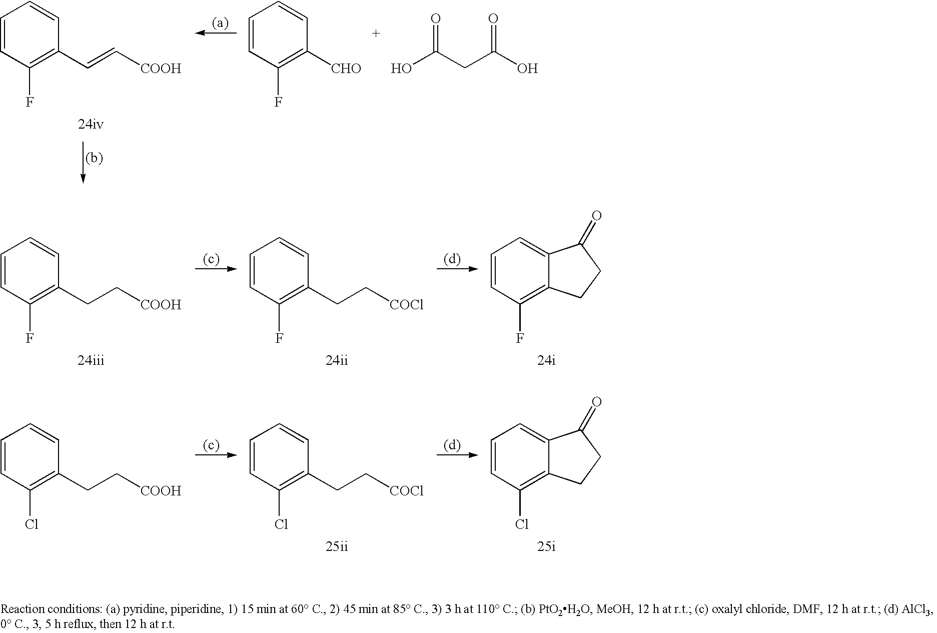

1,3-Thiazole-5-carbaldehyde (27i) was prepared in two steps (Dondoni, A. et al., Synthesis 11: 998-1001 (1987)):

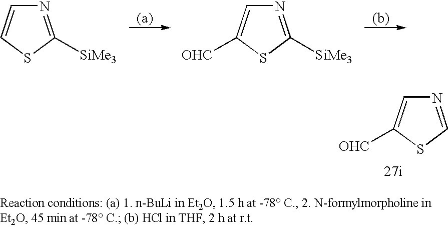

Pyrimidine-5-carbaldehyde (28i) was prepared by analogy with Rho et al. (slightly modified) (Rho, T. & Abuh, Y. F., Synth. Comm. 24: 253-256 (1994)):

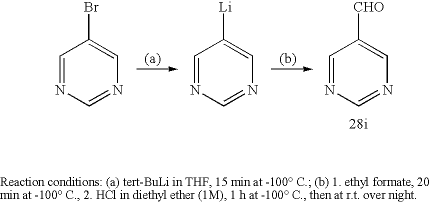

B) Synthesis of compounds 1-38: 50 mmol of ketone was dissolved in a mixture of methanol (100 ml) and THF (100 ml), and 1.89 g of NaBH₄ (50 mmol) was added in small portions with cooling to 0° C. After 10 min at 0° C., the solution was stirred at room temperature (r.t.) for 1 h. The product was extracted with water and ethyl ester. The organic phase was washed first with 1 N HCl, then with a saturated NaHCO₃ solution and finally with water. After drying over MgSO₄, the solvent was removed in vacuo.

The thus obtained alcohol was then directly converted to the phosphonium salt. Thus, 40 mmol of the alcohol and 13.7 g of triphenylphosphonium bromide (40 mmol) (Hercouet, A. & Le Corre, M., Synth. Comm. 157-158 (1988)) was suspended in 50 ml of benzene and refluxed for 12 h under a nitrogen atmosphere. The precipitate was filtered off and dried. The solid was suspended in dry diethyl ether and stirred for 10 min. The phosphonium salt was filtered off and washed with diethyl ether.

For the synthesis of the title compounds, a suspension of 5 mmol of phosphonium salt, 5 mmol of the heterocyclic carbonyl compound (nicotinaldehyde, isonicotinaldehyde, 27i, 28i, quinoline-4-carbaldehyde, quinoline-5-carbaldehyde, isoquinoline-4-carbaldehyde or 1-pyridine-3-ylethanone), 50 mmol of K₂CO₃ and 150-200 mg of 18-crown-6 in 25 ml of dry CH₂Cl₂ was refluxed for 12 h under a nitrogen atmosphere. The reaction mixture was poured into water and repeatedly extracted with CH₂Cl₂. The combined organic phases were dried over MgSO₄, and the solvent was removed in vacuo. After purification with chromatographic methods, the free base was either dissolved in acetone and admixed with an excess of oxalic acid in acetone to obtain the oxalate, or it was dissolved in dry diethyl ether and admixed with an excess of HCl in diethyl ether to obtain the hydrochloride.

In most cases, this synthesis yields E and Z isomers, only with substituents in 7-position on the aromatic ring of the indane or tetraline nucleus, only the non-sterically hindered E isomer was formed. The yields were up to 90%.

### C) Purification Conditions, Yield and Characterization of the Title Compounds:

### 3-[(E)-2,3-Dihydro-1H-indane-1-ylidenemethyl]pyridine Hydrochloride (1a)

Purification: Flash column chromatography (FCC) (EtOAc:hexane, 1:1). Yield 53%, white solid, m.p. 235° C. ¹H NMR (500 MHz, DMSO-d₆) δ 3.10-3.15 (m, 4H, H-2, H-3); 7.20 (s, 1H, H-8); 7.33-7.35 (m, 2H, H-5, H-6); 7.39-7.41 (m, 1H, H-4); 7.76-7.78 (m, 1H, H-7); 7.89-7.92 (m, 1H, H-13); 8.45 (d, ³J=8.2 Hz, 1H, H-14); 8.65 (dd, ³J=5.4 Hz, ⁴J=1.3 Hz, 1H, H-12); 8.90 (s, 1H, H-10). IR cm⁻¹: νₘₐₓ 2411, 1636, 1551, 1471, 825, 806, 751. Anal. (C₁₅H₁₃N.HCl.0.3H₂O) C, H, N.

### 3-[(Z)-2,3-Dihydro-1H-indane-1-ylidenemethyl]pyridine Hydrochloride (1b)

Purification: FCC (EtOAc:hexane, 1:1). Yield 22%, white solid, m.p. 229° C. ¹H NMR (500 MHz, DMSO-d₆) δ 2.91-2.95 (m, 2H, H-2); 2.98-3.01 (m, 2H, H-3); 6.63 (s, 1H, H-8); 7.00-7.06 (m, 2H, H-5, H-6); 7.25-7.40 (m, 2H, H-4, H-7); 7.88 (dd, ³J=5.5 Hz, ³J=8.0 Hz, 1H, H-13); 8.34 (d, ³J=7.4 Hz, 1H, H-14); 8.73 (d, ³J=5.4 Hz, 1H, H-12); 8.81 (s, 1H, H-10). IR cm⁻¹: νₘₐₓ 3022, 2934, 2841, 2427, 1609, 1548, 1455, 1016, 902, 815, 760, 749. Anal. (C₁₅H₁₃N.HCl) C, H, N.

### 4-[(E)-2,3-Dihydro-1H-indane-1-ylidenemethyl]pyridine Oxalate (2a)

Purification: FCC (acetone:petrol ether, 3:10). Yield 33%, yellow solid, m.p. 167° C. ¹H NMR (400 MHz, DMSO-d₆) δ 3.10-3.15 (m, 4H, H-2, H-3); 7.10 (s, 1H, H-8); 7.29-7.40 (m, 3H, H-4, H-5, H-6); 7.55 (d, ³J=6.1 Hz, 2H, H-10, H-14); 7.78-7.80 (m, 1H, H-7); 8.59 (d, ³J=6.1 Hz, 2H, H-11, H-13). IR (KBr) cm⁻¹: νₘₐₓ 1660, 1610, 1510, 900, 830, 810, 760, 750, 730, 710. Anal. (C₁₅H₁₃N.C₂H₂O₄) C, H, N.

### 4-[(Z)-2,3-Dihydro-1H-indane-1-ylidenemethyl]pyridine oxalate (2b)

Purification: FCC (acetone:petrol ether, 3:10). Yield 22%, light yellow solid, m.p. 140° C. ¹H NMR (400 MHz, DMSO-d₆) δ 2.88-2.91 (m, 2H, H-2); 2.95-2.99 (m, 2H, H-3); 6.58 (s, 1H, H-8); 7.03 (t, ³J=7.7 Hz, 1H, H-5); 7.19-7.26 (m, 2H, H-4, H-6); 7.35 (d, ³J=7.6 Hz, 1H, H-7); 7.40 (d, ³J=6.0 Hz, 2H, H-10, H-14); 8.57 (d, ³J=6.0 Hz, 2H, H-11, H-13). IR (KBr) cm⁻¹: νₘₐₓ 3080, 3040, 1610, 1500, 900, 840, 820, 760, 750, 700; Anal. (C₁₅H₁₃N.C₂H₂O₄) C, H, N.

### 3-[(E)-3,4-Dihydronaphthalene-1(2H)-ylidenemethyl]pyridine Hydrochloride (3a)

Purification: FCC (EtOAc:hexane, 1:1). Yield 46%, white solid, m.p. 209° C. ¹H NMR (500 MHz, DMSO-d₆) δ 1.76-1.81 (m, 2H, H-3); 2.77-2.83 (m, 4H, H-2, H-4); 7.19-7.29 (m, 4H, H-5, H-6, H-7, H-8); 7.83-7.85 (m, 1H, H-14); 7.97 (s, 1H, H-9); 8.48 (d, ³J=8.2 Hz, 1H, H-15); 8.73 (d, ³J=5.7 Hz, 1H, H-13); 8.90 (s, 1H, H-11). IR cm⁻¹: νₘₐₓ 3056, 3019, 2953, 2278, 1621, 1569, 1460, 1351, 860, 818, 789, 756. Anal. (C₁₆H₁₅N.HCl) C, H, N.

### 3-[(Z)-3,4-Dihydronaphthalene-1(2H)-ylidenemethyl]pyridine Hydrochloride (3b)

Purification: FCC (EtOAc:hexane, 1:1). Yield 18%, white solid, m.p. 206° C. ¹H NMR (500 MHz, DMSO-d₆) δ 1.98 (m, ³J=6.6 Hz, 2H, H-3); 2.55 (t, ³J=6.6 Hz, 2H, H-2); 2.88 (t, ³J=6.7 Hz, 2H, H-4); 6.52 (s, 1H, H-9); 6.88-6.96 (m, 2H, H-6, H-7); 7.18-7.24 (m, 2H, H-5, H-8); 7.74 (d, ³J=8.1 Hz, 1H, H-14); 8.31 (d, ³J=8.3 Hz, 1H, H-15); 8.59-8.62 (m, 2H, H-13, H-11). IR cm⁻¹: νₘₐₓ 3046, 2936, 1524, 1458, 813, 757. Anal. (C₁₆H₁₅N.HCl) C, H, N.

### 4-[(E)-3,4-Dihydronaphthaliene-1(2H)-ylidenemethyl]pyridine (4a)

Purification: Recrystallization from hexane. Yield 43%, light green crystals, m.p. 66° C. ¹H NMR (400 MHz, DMSO-d₆) δ 1.63-1.69 (m, 2H, H-3); 2.78-2.86 (m, 4H, H-2, H-4); 6.92 (s, 1H, H-9); 7.13-7.15 (m, 1H, H-7); 7.20-7.26 (m, 4H, H-5, H-6, H-11, H-15); 7.68-7.71 (m, 1H, H-8); 8.58 (dd, ³J=4.7 Hz, ⁴J=1.4 Hz, 2H, H-12, H-14). IR (KBr) cm⁻¹: νₘₐₓ 3080, 3040, 1610, 1500, 840, 820, 760, 750, 700; Anal. (C₁₆H₁₅N) C, H, N.

### 3-[(E)-(5-Fluoro-2,3-dihydro-1H-indane-1-ylidene)methyl]pyridine Hydrochloride (5a)

Purification: FCC (EtOAc:hexane, 1:5). Yield 24%, white solid, m.p. 245° C. ¹H NMR (500 MHz, DMSO-d₆) δ 3.11-3.14 (m, 2H, H-2); 3.16-3.20 (m, 2H, H-3); 7.15-7.19 (m, 2H, H-8, H-6); 7.24 (dd, ³J=8.9 Hz, ⁴J=2.5 Hz, 1H, H-4); 7.80 (dd, ³J=8.5 Hz, ⁴J=5.3 Hz, 1H, H-7). 7.94 (dd, ³J=8.2 Hz, ³J=5.3 Hz, 1H, H-13); 8.49 (d, ³J=7.9 Hz, 1H, H-14); 8.68 (d, ³J=5.3 Hz, 1H, H-12); 8.90 (s, 1H, H-10). IR cm⁻¹: νₘₐₓ 3017, 2399, 1637, 1591, 1484, 1240, 936, 859. Anal. (C₁₅H₁₂NF.HCl.0.5H₂O) C, N, H: calc. 5.21. found 4.59.

### 3-[(Z)-(5-Fluoro-2,3-dihydro-1H-indane-1-ylidene)methyl]pyridine Hydrochloride (5b)

Purification: FCC (EtOAc:hexane, 1:5). Yield 19%, beige solid, m.p. 245° C. ¹H NMR (500 MHz, DMSO-d₆) δ 3.11-3.14 (m, 2H, H-2); 3.16-3.19 (m, 2H, H-3); 6.27 (s, 1H, H-8); 7.08-7.13 (m, 1H, H-6); 7.35 (dd, ³J=8.7 Hz, ⁴J=2.5 Hz, 1H, H-4); 7.39 (dd, ³J=8.3 Hz, ⁴J=5.2 Hz, 1H, H-7); 7.94 (m, 1H, H-13); 8.42 (d, ³J=7.9 Hz, 1H, H-14); 8.77 (d, ³J=5.4 Hz, 1H, H-12), 8.91 (s, 1H, H-10). IR cm⁻¹: νₘₐₓ 3050, 3014, 2395, 1552, 1244, 1224, 933, 858, 813, 705. Anal. (C₁₅H₁₂NF.HCl) H, N, C: calc. 68.84. found 68.27.

### 4-[(E)-(5-Fluoro-2,3-dihydro-1H-indane-1-ylidene)methyl]pyridine Hydrochloride (6a)

Purification: FCC (EtOAc:hexane, 2:3). Yield 15%, yellow solid, m.p. 224° C. ¹H NMR (500 MHz, DMSO-d₆) δ 3.17-3.19 (m, 2H, H-2); 3.27-3.30 (m, 2H, H-3); 7.24 (dt, ³J=8.9 Hz, ⁴J=2.5 Hz, 1H, H-6); 7.31 (dd, ³J=8.9 Hz, ⁴J=2.5 Hz, 1H, H-4); 7.33 (s, 1H, H-8); 7.94 (dd, ³J=8.5 Hz, ⁴J (H,F)=5.3 Hz, 1H, H-7); 8.00 (d, ³J=6.9 Hz, 2H, H-10, H-14); 8.78 (d, ³J=6.6 Hz, 2H, H-11, H-13). IR cm⁻¹: νₘₐₓ 2361, 1583, 1511, 1247, 1209, 833, 805. Anal. (C₁₅H₁₂NF.HCl.0.5H₂O) C, H, N.

### 4-[(Z)-(5-Fluoro-2,3-dihydro-1H-indane-1-ylidene)methyl]pyridine Hydrochloride (6b)

Purification: FCC (EtOAc:hexane, 2:3). Yield 18%, yellow solid, m.p. 223° C. ¹H NMR (500 MHz, DMSO-d₆) δ 3.17-3.19 (m, 2H, H-2); 3.26-3.30 (m, 2H, H-3); 6.38 (s, 1H, H-8); 7.10 (dt, ³J=8.5 Hz, ⁴J=2.5 Hz, 1H, H-6); 7.33 (m, 2H, H-4, H-7); 7.98 (m, 2H, H-10, H-14); 8.81 (d, ³J=6.9 Hz, 2H, H-11, H-13). IR cm⁻¹: νₘₐₓ 3046, 2646, 1596, 1510, 1497, 1331, 1248, 1210, 860, 818, 808. Anal. (C₁₅H₁₂NF.HCl.0.3H₂O) C, H, N.

### 3-[(E)-(5-Chloro-2.3-dihydro-1H-indane-1-ylidene)methyl]pyridine Hydrochloride (7a)

Purification: FCC (EtOAc:hexane, 1:1). Yield 52%, white solid, m.p. 234° C. ¹H NMR (500 MHz, DMSO-d₆) δ 3.11-3.18 (m, 4H, H-2, H-3); 7.25 (s, 1H, H-8); 7.39 (dd, ³J=8.4 Hz, ⁴J=2.0 Hz, 1H, H-6); 7.48 (s, 1H, H-4); 7.78 (d, ³J=8.5 Hz, 1H, H-7); 7.97 (m, 1H, H-13); 8.53 (d, ³J=8.2 Hz, H-14); 8.70 (d, ³J=5.4 Hz, 1H, H-12); 8.93 (s, 1H, H-10). IR cm⁻¹: νₘₐₓ 3087, 2924, 2377, 1635, 1548, 1201, 1179, 1073, 919, 864, 825, 801. Anal. (C₁₅H₁₂NCl.HCl) C, H, N.

### 3-[(Z)-(5-Chloro-2,3-dihydro-1H-indane-1-ylidene)methyl]pyridine Hydrochloride (7b)

Purification: FCC (EtOAc:hexane, 1:5). Yield 21%, white solid, m.p. 238° C. ¹H NMR (500 MHz, DMSO-d₆) δ 3.08-3.15 (m, 4H, H-2, H-3); 6.91 (dd, ³J=8.5 Hz, ⁴J=2.4 Hz, 1H, H-6); 6.97 (d, ⁴J=2.2 Hz, 1H, H-4); 7.05 (s, 1H, H-8); 7.68 (d, ³J=8.5 Hz, 1H, H-7). 7.96 (dd, ³J=8.2 Hz, ³J=5.6 Hz, 1H, H-13); 8.51 (d, ³J=8.5 Hz, H-14); 8.65 (d, ³J=5.4 Hz, 1H, H-12); 8.88 (d, ⁴J=1.9 Hz, 1H, H-10). IR cm⁻¹: νₘₐₓ 3003, 2955, 2630, 1619, 1590, 1499, 1199, 1189, 1072, 875, 815, 790, 750. Anal. (C₁₅H₁₂NCl.HCl.0.3H₂O) C, H, N.

### 4-[(E)-(5-Chloro-2,3-dihydro-1H-indane-1-ylidene)methyl]pyridine Hydrochloride (8a)

Purification: FCC (EtOAc:hexane, 1:1). Yield 29%, yellow solid, m.p. 213° C. ¹H NMR (500 MHz, DMSO-d₆) δ 3.17-3.19 (m, 2H, H-2); 3.27-3.29 (m, 2H, H-3); 7.39 (t, ⁴J=2.5 Hz, 1H, H-8); 7.44 (m, 1H, H-6); 7.55 (d, ⁴J=1.6 Hz, 1H, H-4); 7.91 (d, ³J=8.5 Hz, 1H, H-7). 7.02 (d, ³J=6.8 Hz, 2H, H-10, H-14); 8.79 (d, ³J=6.8 Hz, 2H, H-11, H-13). IR cm⁻¹: νₘₐₓ 2359, 1589, 1498, 1268, 1198, 897, 877, 827, 803, 753. Anal. (C₁₅H₁₂NCl.HCl.1.6H₂O) C, H, N.

### 4-[(Z)-(5-Chloro-2,3-dihydro-1H-indane-1-ylidene)methyl]pyridine Hydrochloride (8b)

Purification: FCC (EtOAc:hexane, 1:1). Yield 19%, white solid, m.p. 200° C. ¹H NMR (500 MHz, DMSO-d₆) δ 2.96-3.04 (m, 4H, H-2, H-3); 6.76 (s, 1H, H-8); 7.13 (dd, ³J=8.4 Hz, ⁴J=2.1 Hz, 1H, H-6); 7.40 (d, ³J=8.2 Hz, 1H, H-7); 7.50 (s, 1H, H-4); 7.95 (d, ³J=6.6 Hz, 2H, H-10, H-14); 8.79 (d, ³J=6.6 Hz, H-11, H-13). IR cm⁻¹: νₘₐₓ 3066, 2955, 2630, 1619, 1590, 1499, 1199, 1189, 1072, 875, 815, 790, 750. Anal. (C₁₅H₁₂NCI.HCl.0.4H₂O) C, H, N.

### 3-[(E)-(5-Bromo-2,3-dihydro-1H-indane-1-ylidene)methyl]pyridine Hydrochloride (9a)

Purification: FCC (EtOAc:hexane, 1:1). Yield 30%, white solid, m.p. 241° C. ¹H NMR (500 MHz, DMSO-d₆) δ 3.10-3.17 (m, 4H, H-2, H-3); 7.26 (s, 1H, H-8); 7.52 (dd, ³J=8.2 Hz, ⁴J=1.8 Hz, 1H, H-6); 7.62 (d, ⁴J=1.6 Hz, 1H, H-4); 7.71 (d, ³J=8.2 Hz, 1H, H-7); 7.94 (dd, ³J=8.2 Hz, ³J=5.3 Hz, 1H, H-13); 8.49 (d, ³J=8.5 Hz, 1H, H-14); 8.69 (dd, ³J=5.4 Hz, ⁴J=1.3 Hz, 1H, H-12); 8.91 (d, ⁴J=1.6 Hz, 1H, H-10). IR cm⁻¹: νₘₐₓ 3086, 2389, 1635, 1548, 1529, 1065, 919, 861, 822, 802. Anal. (C₁₅H₁₂NBr.HCl) C, H, N.

### 3-[(Z)-(5-Bromo-2,3-dihydro-1H-indane-1-ylidene)methyl]pyridine Hydrochloride (9b)

Purification: FCC (EtOAc:hexane, 1:5). Yield 18%, white solid, m.p. 243° C. ¹H NMR (500 MHz, DMSO-d₆) δ 3.41 (m, 2H, H-2); 4.08 (m, 2H, H-3); 6.30 (s, 1H, H-8); 7.34 (d, ³J=8.2 Hz, 1H, H-7); 7.46 (dd, ³J=8.0 Hz, ⁴J=1.7 Hz, 1H, H-6); 7.68 (d, ⁴J=1.6 Hz, 1H, H-4). 7.84 (dd, ³J=8.0 Hz, ³J=5.5 Hz, 1H, H-13); 8.29 (d, ³J=7.9 Hz, 1H, H-14); 8.72 (d, 3J=5.3 Hz, 1H, H-12); 8.84 (s, 1H, H-10). IR cm⁻¹: νₘₐₓ 3069, 3003, 2515, 2361, 1558, 965, 910, 844, 817. Anal. (C₁₅H₁₂NBr.HCl) C, H, N.

### 4-[(E)-(5-Bromo-2,3-dihydro-1H-indane-1-ylidene)methyl]pyridine Hydrochloride (10a)

Purification: FCC (EtOAc:hexane, 1:1). Yield 28%, yellow solid, m.p. 266° C. ¹H NMR (500 MHz, DMSO-d₆) δ 3.16-3.19 (m, 2H, H-2); 3.24-3.27 (m, 2H, H-3); 7.38 (t, ⁴J=2.2 Hz, 1H, H-8); 7.57 (dd, ³J=8.5 Hz, ⁴J=1.9 Hz, 1H, H-6); 7.69 (s, 1H, H-4); 7.83 (d, 3J=8.2 Hz, 1H, H-7); 7.97 (d, ³J=6.6 Hz, 2H, H-10, H-14); 8.77 (d, ³J=6.6 Hz, 2H, H-11, H-13). IR cm⁻¹: νₘₐₓ 2702, 1608, 1586, 1509, 1199, 828, 807. Anal. (C₁₅H₁₂NBr.HCl) C, H, N.

### 4-[(Z)-(5-Bromo-2,3-dihydro-1H-indane-1-ylidene)methyl]pyridine Hydrochloride (10b)

Purification: FCC (EtOAc:hexane, 1:1). Yield 19%, yellow solid, m.p. 256° C. ¹H NMR (500 MHz, DMSO-d₆) δ 2.96-2.98 (m, 2H, H-2); 3.01-3.04 (m, 2H, H-3); 6.75 (s, 1H, H-8); 7.57 (m, 1H, H-6); 7.65 (s, 1H, H-4); 7.83 (d, ³J=8.2 Hz, 1H, H-7); 7.91 (d, ³J=6.6 Hz, 2H, H-10, H-14); 8.78 (d, ³J=6.8 Hz, 2H, H-11, H-13). IR cm⁻¹: νₘₐₓ 2481, 1625, 1608, 1587, 1507, 1497, 119, 864, 820. Anal. (C₁₅H₁₂NBr.HCl) C, H, N.

### 3-[(E)-(5-Methoxy-2,3-dihydro-1H-indane-1-ylidene)methyl]pyridine Hydrochloride (11a)

Purification: FCC (EtOAc:hexane, 1:1). Yield 42%, light yellow solid, m.p. 230° C. ¹H NMR (500 MHz, DMSO-d₆) δ 3.08-3.15 (m, 4H, H-2, H-3); 3.80 (s, 3H, OCH₃); 6.91 (dd, 3=8.5 Hz, ⁴J=2.5 Hz, 1H, H-6); 6.97 (d, ⁴J=2.5 Hz, 1H, H-4); 7.05 (t, ⁴J=2.2 Hz, 1H, H-8); 7.68 (d, ³J=8.5 Hz, 1H, H-7); 7.96 (dd, ³J=8.2 Hz, ³J=5.7 Hz, H-13); 8.51 (d, ³J=8.5 Hz, 1H, H-14); 8.65 (d, ³J=5.4 Hz, 1H, H-12); 8.89 (s, 1H, H-10). IR cm⁻¹: νₘₐₓ 2838, 2362, 1632, 1602, 1545, 1490, 1310, 1258, 1227, 1110, 833, 798. Anal. (C₁₆H₁₅₀N.HCl) C, H, N.

### 3-[(Z)-(5-Methoxy-2,3-dihydro-1H-indane-1-ylidene)methyl]pyridine Hydrochloride (11b)

Purification: FCC (EtOAc:hexane, 1:5). Yield 18%, yellow solid, m.p. 220° C. ¹H NMR (500 MHz, DMSO-d₆) δ 3.08-3.15 (m, 4H, H-2, H-3); 3.80 (s, 3H, OCH₃); 6.91 (dd, ³J=8.5 Hz, ⁴J=2.5 Hz, 1H, H-6); 6.97 (d, ⁴J=2.4 Hz, 1H, H-4); 7.04 (s, 1H, H-8); 7.68 (d, ³J=8.8 Hz, 1H, H-7); 7.94 (dd, ³J=8.2 Hz, ³J=5.7 Hz, H-13); 8.49 (d, ³J=8.2 Hz, 1H, H-14); 8.64 (d, ³J=5.7 Hz, 1H, H-12); 8.87 (d, ⁴J=1.9 Hz, 1H, H-10). IR cm⁻¹: νₘₐₓ 2844, 2361, 1632, 1602, 1545, 1489, 1309, 1256, 1227, 1109, 1021, 832, 797. Anal. (C₁₆H₁₅ON.HCl.H₂O) C, N, H: calc. 6.22. found 5.34.

### 4-[(E)-(5-Methoxy-2,3-dihydro-1H-indane-1-ylidene)methyl]pyridine Hydrochloride (12a)

Purification: FCC (EtOAc:hexane, 1:1). Yield 250%, yellow solid, m.p. 243° C. ¹H NMR (500 MHz, DMSO-d₆) δ 3.14-3.16 (m, 2H, H-2); 3.24-3.27 (m, 2H, H-3); 3.83 (s, 3H, OCH₃); 6.96 (dd, ³J=8.8 Hz, ⁴J=2.2 Hz, 1H, H-6); 7.03 (d, ⁴J=2.2 Hz, 1H, H-4); 7.19 (t, ⁴J=2.2 Hz, 1H, H-8); 7.81 (d, ³J=8.8 Hz, 1H, H-7); 7.92 (d, ³J=6.9 Hz, 2H, H-10, H-14); 8.71 (d, ³J=6.9 Hz, 2H, H-11, H-13). IR cm⁻¹: νₘₐₓ 2834, 2427, 1580, 1501, 1249, 1092, 825, 802. Anal. (C₁₆H₁₅ON.HCl) C, H, N.

### 4-[(Z)-(5-Methoxy-2,3-dihydro-1H-indane-1-ylidene)methyl]pyridine Hydrochloride (12b)

Purification: FCC (EtOAc:hexane, 1:1). Yield 18%, yellow solid, m.p. 238° C. ¹H NMR (500 MHz, DMSO-d₆) δ 3.14-3.16 (m, 2H, H-2); 3.24-3.27 (m, 2H, H-3); 3.83 (s, 3H, OCH₃); 6.97 (dd, ³J=8.7 Hz, ⁴J=2.2 Hz, 1H, H-6); 7.03 (d, ⁴J=2.2 Hz, 1H, H-4); 7.19 (s, 1H, H-8); 7.82 (d, ³J=8.5 Hz, 1H, H-7); 7.93 (d, ³J=6.9 Hz, 2H, H-10, H-14); 8.71 (d, ³J=6.9 Hz, 2H, H-11, H-13). IR cm⁻¹: νₘₐₓ 2428, 1581, 1502, 1250, 1094, 869, 825, 802. Anal. (C₁₆H₁₅₀N.HCl.0.5H₂O) C, H, N.

### 3-[(E)-(6-Methoxy-3,4-dihydronaphthalene-1(2H)-ylidene)methyl]pyridine Hydrochloride (13a)

Purification: FCC (EtOAc:hexane, 1:1). Yield 36%, yellow solid, m.p. 163° C. ¹H NMR (500 MHz, DMSO-d₆) δ 1.75-1.78 (m, 2H, H-3); 2.73-2.77 (m, 2H, H-2); 2.79-2.81 (m, 2H, H-4); 3.78 (s, 3H, OCH₃); 6.76 (d, ⁴J=2.5 Hz, 1H, H-5); 6.84 (dd, ³J=8.8 Hz, ⁴J=2.8 Hz, 1H, H-7); 7.10 (s, 1H, H-9); 7.79 (d, ³J=8.8 Hz, 1H, H-8); 7.90 (dd, ³J=7.9 Hz, ³J=5.7 Hz, 1H, H-14); 8.40 (d, ³J=8.2 Hz, 1H, H-15); 8.67 (dd, ³J=5.6 Hz, ⁴J=1.3 Hz, 1H, H-13); 8.84 (d, ⁴J=1.6 Hz, 1H, H-11). IR cm⁻¹: νₘₐₓ 2386, 1602, 1586, 1543, 1502, 1236, 1124, 1035, 899, 848, 833, 801. Anal. (C₁₇H₁₇₀N.HCl) C, H, N.

### 3-[(Z)-(6-Methoxy-3,4-dihydronaphthalene-1(2H)-ylidene)methyl]pyridine Hydrochloride (13b)

Purification: FCC (EtOAc:hexane, 1:5). Yield 4%, beige solid, m.p. 151° C. ¹H NMR (500 MHz, DMSO-d₆) δ 2.22-2.26 (m, 2H, H-3); 2.69-2.72 (m, 2H, H-2); 3.71 (s, 3H, OCH₃); 5.80 (s, 1H, H-9); 3.92 (m, 2H, H-4); 6.67 (dd, ³J=8.5 Hz, ⁴J=2.7 Hz, 1H, H-7); 6.77 (d, ³J=2.8 Hz, 1H, H-8); 7.14 (d, ³J=8.5 Hz, 1H, H-5); 7.76 (m, 1H, H-13); 8.18 (m, 1H, H-14); 8.65 (m, 1H, H-12); 8.75 (s, 1H, H-10). IR cm⁻¹: νₘₐₓ 2360, 2342, 1609, 1554, 1496, 1249, 1139, 823, 804, 787. Anal. (C₁₇H₁₇ON.HCl.0.5H₂O) C, H, N.

### 4-[(E)-(6-Methoxy-3,4-dihydronaphthalene-1(2H)-ylidene)methyl]pyridine Hydrochloride (14a)

Purification: FCC (EtOAc:hexane, 1:1). Yield ⁷⁹%, yellow solid, m.p. 173° C. ¹H NMR (500 MHz, DMSO-d₆) δ 1.80 (m, 2H, H-3); 2.82 (t, ³J=6.2 Hz, 2H, H-2); 2.89 (t, ³J=5.4 Hz, 2H, H-4); 3.80 (s, 3, OCH₃); 6.80 (d, ⁴J=2.5 Hz, 1H, H-5); 6.87 (dd, ³J=8.8 Hz, ⁴J=2.5 Hz, 1H, H-7); 7.23 (s, 1H, H-9); 7.89 (d, ³J=8.8 Hz, 1H, H-8); 7.94 (d, ³J=6.6 Hz, 2H, H-11, H-15); 8.75 (d, ³J=6.9 Hz, 2H, H-12, H-14). IR cm⁻¹: νₘₐₓ 3044, 1943, 2843, 2429, 1626, 1504, 1258, 1234, 1189, 1178, 1032, 881, 853, 831, 809. Anal. (C₁₇H₁₇₀N.HCl.0.3H₂O) C, H, N.

### 4-[(Z)-(6-Methoxy-3,4-dihydronaphthalene-1(2H)-ylidene)methyl]pyridine Hydrochloride (14b)

Purification: FCC (EtOAc:hexane, 1:1). Yield 100%, yellow solid, m.p. 198° C. ¹H NMR (500 MHz, DMSO-d₆) δ 2.28 (m, 2H, H-3); 2.74 (t, ³J=8.0 Hz, 2H, H-2); 3.71 (s, 3H, OCH₃); 4.06 (m, 2H, H-4); 5.91 (t, ⁴J=4.5 Hz, 1H, H-9); 6.65 (dd, 1H, ³J=8.5 Hz, ⁴J=2.5 Hz, H-7); 6.77 (d, ⁴J=2.5 Hz, 1H, H-5); 7.05 (d, ³J=8.5 Hz, 1H, H-8); 7.82 (d, ³J=6.6 Hz, 2H, H-11, H-15); 8.73 (d, ³J=6.3 Hz, 2H, H-12, H-14). IR cm⁻¹: νₘₐₓ 3041, 2935, 2823, 1631, 1595, 1565, 1494, 1253, 1139, 820, 797. Anal. (C₁₇H₁₇₀N.HCl.0.6H₂O) C, H, N.

### 3-[(E)-(6-Methoxy-2,3-dihydro-1H-indane-1-ylidene)methyl]pyridine Hydrochloride (15a)

Purification: FCC (EtOAc:hexane, 1:1). Yield 19%, white solid, m.p. 222° C. ¹H NMR (500 MHz, DMSO-d₆) δ 3.02-3.05 (m, 2H, H-2); 3.14-3.17 (m, 2H, H-3); 3.82 (s, 3H, OCH₃); 6.94 (dd, ³J=8.4 Hz, ⁴J=2.4 Hz, 1H, H-6); 7.23 (t, ⁴J=2.5 Hz, 1H, H-8); 7.29 (d, ³J=8.2 Hz, 1H, H-4); 7.32 (d, ⁴J=2.5 Hz, 1H, H-7); 7.93 (dd, ³J=8.2 Hz, ³J=5.5 Hz, 1H, H-13); 8.49 (d, ³J=8.2 Hz, 1H, H-14); 8.67 (d, ³J=5.6 Hz, 1H, H-12); 8.90 (d, ⁴J=1.9 Hz, 1H, H-10) IR cm⁻¹: νₘₐₓ 2980, 2846, 2643, 1636, 1606, 1555, 1489, 1298, 1283, 1222, 1026, 908, 867, 796. Anal. (C₁₆H₁₅ON.HCl.0.4H₂O) C, H, N.

### 4-[(E)-(6-Methoxy-2,3-dihydro-1H-indane-1-ylidene)methyl]pyridine Hydrochloride (16a)

Purification: FCC (EtOAc:hexane, 1:1). Yield 20%, yellow solid, m.p. 220° C. ¹H NMR (500 MHz, DMSO-d₆) δ 3.09-3.11 (m, 2H, H-2); 3.27-3.28 (m, 2H, H-3); 3.83 (s, 3H, OCH₃); 7.04 (dd, ³J=8.4 Hz, ⁴J=2.4 Hz, 1H, H-5); 7.36 (d, ³J=8.5 Hz, 1H, H-4); 7.40 (s, 1H, H-8); 7.45 (d, ⁴J=2.5 Hz, 1H, H-7); 8.01 (d, ³J=6.9 Hz, 2H, H-10, H-14); 8.79 (d, ³J=8.5 Hz, 2H, H-11, H-13). IR cm⁻¹: νₘₐₓ 3051, 3001, 2736, 1604, 1508, 1497, 1222, 1200, 1020, 893, 806. Anal. (C₁₆H₁₅ON.HCl.0.8H₂O) C, H, N.

### 4-[(Z)-(6-Methoxy-2,3-dihydro-1H-indane-1-ylidene)methyl]pyridine Hydrochloride (16b)

Purification: FCC (EtOAc:hexane, 1:1). Yield 13%, yellow solid, m.p. 207° C. ¹H NMR (500 MHz, DMSO-d₆) δ 3.03-3.05 (m, 2H, H-2); 3.20-3.27 (m, 2H, H-2); 3.78 (s, 3H, OCH₃); 6.35 (s, 1H, H-8); 6.73 (d, ³J=8.2 Hz, 1H, H-5); 6.98 (d, ³J=8.2 Hz, 1H, H-4); 7.30 (s, 1H, H-7); 7.91-7.96 (d, ³J=7.0 Hz, 2H, H-10, H-14); 8.72-8.77 (d, ³J=7.0 Hz, 2H, H-11, H-13). IR cm⁻¹: νₘₐₓ 3005, 2946, 2359, 1638, 1609, 1507, 1475, 1289, 1219, 1178, 1026, 808. Anal. (C₁₆H₁₅ON.HCl) C, H, N.

### 3-[(E)-(6,7-Dimethoxy-3,4-dihydronaphthalene-1(2H)-ylidene)methyl]pyridine Hydrochloride (17a)

Purification: FCC (EtOAc:hexane, 1:1). Yield 65%, yellow solid, m.p. 197° C. ¹H NMR (500 MHz, DMSO-d₆) δ 1.74-1.79 (m, 2H, H-3); 2.73-2.77 (m, 4H, H-2, H-4); 3.78 (s, 3H, OCH₃); 3.82 (s, 3H, OCH₃); 6.77 (s, 1H, H-9); 7.19 (s, 1H, H-5); 7.37 (s, 1H, H-8); 8.03 (m, 1H, H-13); 8.55 (m, 1H, H-14); 8.74 (d, ³J=5.4 Hz, 1H, H-12); 8.92 (s, 1H, H-10). IR cm⁻¹: νₘₐₓ 2931, 2835, 2419, 1714, 1603, 1586, 1550, 1514, 1466, 1454, 1254, 1215, 1139, 1028, 1016, 871, 855, 833, 800. Anal. (C₁₈H₁₉O₂N.HCl 0.8H₂O) C, H, N.

### 4-[(E)-(6,7-Dimethoxy-3,4-dihydronaphthalene-1(2H)-ylidene)methyl]pyridine Hydrochloride (18a)

Purification: FCC (EtOAc:hexane, 1:1). Yield 68%, yellow solid, m.p. 197° C. ¹H NMR (500 MHz, DMSO-d₆) δ 1.77-1.82 (m, 2H, H-3); 2.77-2.79 (m, 2H, H-2); 2.86-2.89 (m, 2H, H-4); 3.80 (s, 3H, OCH₃); 3.83 (s, 3H, OCH₃); 6.81 (s, 1H, H-9); 7.09 (s, 1H, H-5); 7.43 (s, 1H, H-8); 8.00 (d, ³J=6.8 Hz, 2H, H-11, H-15); 8.78 (d, ³J=6.8 Hz, 2H, H-12, H-14). IR cm⁻¹: νₘₐₓ 3027, 2930, 2360, 1627, 1597, 1571, 1503, 1358, 1257, 1218, 1192, 1141, 1025, 872, 844, 786. Anal. (C₁₈H₁₉O₂N.HCl.1.1H₂O) C, H, N.

### 3-[(E)-(5-Ethoxy-2,3-dihydro-1H-indane-1-ylidene)methyl]pyridine Hydrochloride (19a)

Prepared from (19i). Purification: FCC (EtOAc:hexane, 1:1). Yield 43%, yellow solid, m.p. 225° C. ¹H NMR (500 MHz, DMSO-d₆) δ 1.34 (t, ³J=6.9 Hz, 3H, OCH₂CH₃); 3.07-3.14 (m, 4H, H-2, H-3); 4.06 (q, ³J=6.9 Hz, 2H, OCH₂CH₃); 6.89 (dd, ³J=8.8 Hz, ⁴J=2.5 Hz, 3H, H-6); 6.95 (s, 1H, H-8); 7.04 (t, ⁴J=2.2 Hz, 1H, H-4); 7.67 (d, ³J=8.5 Hz, 1H, H-7); 7.94 (dd, ³J=8.2 Hz, ³J=5.4 Hz, 1H, H-13); 8.49 (d, ³J=8.2 Hz, 1H, H-14); 8.64 (d, ³J=5.4 Hz, 1H, H-12); 8.88 (s, 1H, H-10). IR cm⁻¹: νₘₐₓ 3057, 3031, 2984, 2595, 1632, 1590, 1550, 1475, 1247, 1093, 1044, 824, 805. Anal. (C₁₇H₁₇ON.HCl.0.3H₂O) C, H, N.

### 3-[(Z)-(5-Ethoxy-2,3-dihydro-1H-indane-1-ylidene)methyl]pyridine Hydrochloride (19b)

Prepared from (19i). Purification: FCC (EtOAc:hexane, 1:1). Yield 11%, yellow solid, m.p. 219° C. ¹H NMR (500 MHz, DMSO-d₆) δ 1.34 (t, ³J=6.9 Hz, 3H, OCH₂CH₃); 3.06-3.14 (m, 4H, H-2, H-3); 4.07 (q, ³J=6.9 Hz, 2H, OCH₂CH₃); 6.89 (dd, ³J=8.8 Hz, ⁴J=2.2 Hz, 3H, H-6); 6.94 (s, 1H, H-8); 7.02 (t, ⁴J=2.2 Hz, 1H, H-4); 7.66 (d, ³J=8.5 Hz, 1H, H-7); 7.89 (dd, ³J=8.5 Hz, ³J=5.0 Hz, 1H, H-13); 8.43 (d, ³J=8.5 Hz, 1H, H-14); 8.62 (s, 1H, H-12); 8.86 (s, 1H, H-10). IR cm⁻¹: νₘₐₓ 3032, 2984, 2921, 2880, 2595, 1632, 1590, 1552, 1475, 1247, 1092, 1045, 825, 806. Anal. (C₁₇H₁₇₀N.HCl.0.2H₂O) C, H, N.

### 3-{(E)-[5-(Benzyloxy)-2,3-dihydro-1H-indane-1-ylidene]methyl}pyridine Hydrochloride (20a)

Prepared from (20i). Purification: FCC (EtOAc:hexane, 1:3). Yield 13%, yellow solid, m.p. 209° C. ¹H NMR (500 MHz, DMSO-d₆) δ 3.08-3.16 (m, 4H, H-2, H-3); 5.16 (s, 2H, OBn); 6.99-7.08 (m, 3H, H-4, H-6, H-8); 7.33-7.47 (m, 5H, OBn); 7.69 (d, ³J=8.8 Hz, 1H, H-7); 8.02 (dd, ³J=8.2 Hz, ³J=5.7 Hz, 1H, H-13); 8.57 (d, ³J=7.9 Hz, 1H, H-14); 8.68 (d, ³J=5.7 Hz, 1H, H-12); 8.91 (s, 1H, H-10).

IR cm⁻¹: νₘₐₓ 3385, 3097, 3033, 2914, 2505, 1624, 1595, 1531, 1243, 1226, 1153, 1091, 996, 829, 774. Anal. (C₂₂H₁₉ON.HCl.0.2H₂O) C, H, N.

### 3-[(E)-(6-Methyl-2,3-dihydro-1H-indane-1-ylidene)methyl]pyridine Hydrochloride (21a)

Prepared from (21i). Purification: FCC (EtOAc:hexane, 1:4). Yield 37%, beige solid, m.p. 245° C. ¹H NMR (500 MHz, DMSO-d₆) δ 2.37 (s, 3H, CH₃); 3.06-3.15 (m, 4H, H-2, H-3); 7.18 (d, ³J=7.6 Hz, H-5); 7.23 (s, 1H, H-8); 7.29 (d, ³J=7.9 Hz, 1H, H-4); 7.59 (s, 1H, H-7); 8.06 (dd, ³J=8.2 Hz, ⁴J=5.5 Hz, 1H, H-13); 8.63 (d, ³J=8.2 Hz, 1H, H-14); 8.73 (d, ³J=5.4 Hz, 1H, H-12); 8.95 (s, 1H, H-10). IR cm⁻¹: νₘₐₓ 2658, 1636, 1600, 1552, 888. Anal. (C₁₆H₁₅N.HCl.0.8H₂O) C, H, N.

### 3-[(Z)-(6-Methyl-2,3-dihydro-1H-indane-1-ylidene)methyl]pyridine Hydrochloride (21b)

Prepared from (21i). Purification: FCC (EtOAc:hexane, 1:5). Yield 9%, white solid, m.p. 241° C. ¹H NMR (500 MHz, DMSO-d₆) δ 2.36 (s, 3H, CH₃); 3.40-3.16 (m, 4H, H-2, H-3); 7.16-7.29 (m, 3H, H-8, H-4, H-5); 7.58 (s, 1H, H-7); 7.96 (dd, ³J=8.2 Hz, ³J=5.7 Hz, 1H, H-13); 8.52 (d, ³J=8.5 Hz, 1H, H-14); 8.68 (d, ³J=5.0 Hz, 1H, H-12); 8.92 (s, 1H, H-10). IR cm⁻¹: νₘₐₓ 2420, 1637, 1617, 1598, 1549, 895, 817, 786. Anal. (C₁₆H₁₅N.HCl.0.3H₂O) C, H, N.

### 4-[(E)-(6-Methyl-2,3-dihydro-1H-indane-1-ylidene)methyl]-pyridine Hydrochloride (22a)

Prepared from (21i). Purification: FCC (EtOAc:hexane, 1:4). Yield 42%, light yellow solid, m.p. 200° C. ¹H NMR (500 MHz, DMSO-d₆) δ 2.38 (s, 3H, CH₃); 3.11-3.15 (m, 2H, H-2); 3.24-3.28 (m, 2H, H-3); 7.27-7.35 (m, 2H, H-4, H-5); 8.03 (d, ³J=6.7 Hz, 2H, H-10, H-14); 8.78 (d, ³J=6.7 Hz, 2H, H-11, H-13); 8.97 (d, ³J=5.8 Hz, 1H, H-7). IR cm⁻¹: νₘₐₓ 1591, 1506, 1496, 887, 794. Anal. (C₁₆H₁₅N.HCl.2.6H₂O) C, H, N.

### 4-[(Z)-(6-Methyl-2,3-dihydro-1H-indane-1-ylidene)methyl]pyridine Hydrochloride (22b)

Prepared from (21i). Purification: FCC (EtOAc:hexane, 1:4). Yield 10%, yellow solid, m.p. 228° C. ¹H NMR (500 MHz, DMSO-d₆) δ 2.38 (s, 3H, CH₃); 3.11-3.14 (m, 2H, H-2); 3.24-3.27 (m, 2H, H-3); 6.69 (s, 1H, H-8); 7.16-7.35 (m, 3H, H-4, H-5, H-7); 7.93 (d, ³J=6.6 Hz, 1H, H-14); 7.99 (d, ³J=6.8 Hz, 1H, H-10); 8.76 (d, ³J=6.6 Hz, 2H, H-11, H-13). IR cm⁻¹: νₘₐₓ 2917, 2460, 1596, 1510, 1204, 880, 813. Anal. (C₁₆H₁₅N.HCl.0.5H₂O) C, H, N.

### 3-[(E)-(4-Methyl-2,3-dihydro-1H-indane-1-ylidene)methyl]pyridine Hydrochloride (23a)

Purification: FCC (EtOAc:hexane, 1:1). Yield 48%, yellow solid, m.p. 231° C. ¹H NMR (500 MHz, DMSO-d₆) δ 2.25 (s, 3H, CH₃); 2.96-3.10 (m, 2H, H-2, H-3); 7.03 (t, ⁴J=2.5 Hz, 1H, H-8); 7.09 (d, ³J=7.3 Hz, 1H, H-5); 7.19 (t, ³J=7.6 Hz, 1H, H-6); 7.41 (dd, ³J=7.8 Hz, ³J=4.7 Hz, 1H, H-13); 7.55 (d, ³J=7.6 Hz, 1H, H-7); 7.90 (d, ³J=8.2 Hz, 1H, H-14); 8.45 (dd, ³J=4.7 Hz, ⁴J=1.6 Hz, 1H, H-12); 8.70 (d, ⁴J=4.4 Hz, 1H, H-10). IR cm⁻¹: νₘₐₓ 3013, 2408, 1635, 1552, 938, 885, 825, 784. Anal. (C₁₆H₁₅N.HCl.0.3H₂O)H, N, C: calc. 74.56. found 75.52.

### 3-[(Z)-(4-Methyl-2,3-dihydro-1H-indane-1-ylidene)methyl]pyridine Hydrochloride (23b)

Purification: FCC (EtOAc:hexane, 1:1). Yield 43%, yellow solid, m.p. 173° C. ¹H NMR (500 MHz, DMSO-d₆) δ 2.25 (s, 3H, CH₃); 2.91 (m, 2H, H-2, H-3); 6.59 (s, 1H, H-8); 6.84 (d, J=7.8 Hz, 1H, H-5); 6.92 (t, ³J=7.8 Hz, 1H, H-6); 7.05 (d, ³J=7.6 Hz, 1H, H-7); 7.35-7.40 (m, 1H, H-13); 7.78 (d, ³J=7.8 Hz, 1H, H-14); 8.51 (d, ³J=4.7 Hz, 1H, H-12); 8.55 (d, ⁴J=2.2 Hz, 1H, H-10). IR cm⁻¹: νₘₐₓ 2982, 2933, 1614, 1232, 856, 764. Anal. (C₁₆H₁₅N.HCl) C, H, N.

### 3-[(E)-(4-Fluoro-2,3-dihydro-1H-indane-1-ylidene)methyl]pyridine Hydrochloride (24a)

Prepared from (24i). Purification: FCC (EtOAc:hexane, 1:1). Yield 20%, yellow solid, m.p. 246° C. ¹H NMR (500 MHz, DMSO-d₆) δ 3.07-3.15 (m, 4H, H-2, H-3); 7.14 (t, ⁴J=2.5 Hz, 1H, H-8); 7.31-7.36 (m, 2H, H-5, H-6); 7.43 (dd, ³J=7.8 Hz, ³J=4.7 Hz, 1H, H-13); 7.72 (dd, ³J=7.3 Hz, ⁴J=1.6 Hz, 1H, H-7); 7.91 (d, ³J=7.8 Hz, 1H, H-14); 8.43 (dd, ³J=4.7 Hz, ⁴J=1.6 Hz, 1H, H-12); 8.71 (d, ⁴J=2.5 Hz, 1H, H-10). IR cm⁻¹: νₘₐₓ 3097, 3062, 2405, 1639, 1551, 1130, 873, 786. Anal. (C₁₆H₁₅NF.HCl.1.4H₂O) C, H, N.

### 3-[(Z)-(4-Fluoro-2,3-dihydro-1H-indane-1-ylidene)methyl]pyridine Hydrochloride (24b)

Prepared from (24i). Purification: FCC (EtOAc:hexane, 1:1). Yield 9%, yellow solid, m.p. 213° C. ¹H NMR (500 MHz, DMSO-d₆) δ 2.94-3.01 (m, 4H, H-2, H-3); 6.68 (s, 1H, H-8); 6.92 (d, ³J=7.6 Hz, 1H, H-5); 7.05 (t, ³J=7.8 Hz, 1H, H-6); 7.30 (d, ³J=7.8 Hz, 1H, H-7); 7.41-7.44 (m, 1H, H-13); 7.75 (d, ³J=7.8 Hz, 1H, H-14); 8.50-8.54 (m, 2H, H-10, H-12). IR cm⁻¹: νₘₐₓ 3075, 2919, 2431, 1727, 1610, 1546, 1455, 1128, 780. Anal. (C₁₆H₁₅NF.HCl.1.4H₂O) C, H, N.

### 3-[(E)-(4-Chloro-2,3-dihydro-1H-indane-1-ylidene)methyl]pyridine Hydrochloride (25a)

Prepared from (25i). Purification: FCC (EtOAc:hexane, 1:1). Yield 35%, white solid, m.p. 244° C. ¹H NMR (500 MHz, DMSO-d₆) δ 2.86-2.94 (m, 4H, H-2, H-3); 6.87 (t, ³J=8.8 Hz, 1H, H-6); 6.92 (s, 1H, H-8); 7.09-7.12 (m, 1H, H-5); 7.21 (dd, ³J=7.8 Hz, ³J=4.7 Hz, 1H, H-13); 7.38 (d, ³J=7.6 Hz, 1H, H-7); 7.70 (d, ³J=7.8 Hz, 1H, H-14); 8.21 (d, ³J=4.7 Hz, 1H, H-12); 8.50 (s, 1H, H-10). IR cm⁻¹: νₘₐₓ 2403, 1553, 1474, 1240, 936, 785. Anal. (C₁₅H₁₂NCl.HCl) C, H, N.

### 3-[(Z)-(4-Chloro-2,3-dihydro-1H-indane-1-ylidene)methyl]pyridine Hydrochloride (25b)

Prepared from (25i). Purification: FCC (EtOAc:hexane, 1:1). Yield 11%, yellow solid, m.p. 208° C. ¹H NMR (500 MHz, DMSO-d₆) δ 2.99 (m, 4H, H-2, H-3); 6.69 (s, 1H, H-8); 6.81 (m, 1H, H-6); 7.06-7.09 (m, 2H, H-5, H-7); 7.43-7.45 (m, 1H, H-13); 7.76 (d, ³J=7.8 Hz, 1H, H-14); 8.52-8.55 (m, 2H, H-10, H-12). IR cm⁻¹: νₘₐₓ 3042, 2394, 1638, 1551, 1473, 1239, 858, 781. Anal. (C₁₅H₁₂NCl.HCl) C, H, N.

### 3-[(E)-(7-Methoxy-2,3-dihydro-1H-indane-1-ylidene)methyl]pyridine Hydrochloride (26a)

Prepared from (26i). Purification: FCC (EtOAc:hexane, 1:1). Yield 90%, yellow solid, m.p. 238° C. ¹H NMR (500 MHz, DMSO-d₆) δ 3.06 (s, 3H, OCH₃); 2.94 (s, 4H, H-2, H-3); 6.94 (d, ³J=8.2 Hz, 1H, H-6); 6.97 (d, ³J=7.3 Hz, 1H, H-4); 7.30 (t, ³J=7.7 Hz, 1H, H-5); 7.54 (s, 1H, H-8); 7.88 (m, 1H, H-13); 8.41 (d, ³J=7.3 Hz, 1H, H-14); 8.62 (d, ³J=5.2 Hz, 1H, H-12); 8.88 (s, 1H, H-10). IR cm⁻¹: νₘₐₓ 2917, 2460, 1596, 1510, 1204, 880, 813. IR cm⁻¹: νₘₐₓ 3003, 2839, 2363, 2083, 1605, 1584, 1481, 1468. 1455, 1303, 1067, 894, 794. Anal. (C₁₆H₁₅ON.HCl.1.2H₂O) C, H, N.

### 5-[(E)-(5-Methoxy-2,3-dihydro-1H-indane-1-ylidene)methyl]-1,3-thiazole Hydrochloride (27a)

Prepared from (27i). Purification: FCC (EtOAc:hexane, 1:3). Yield 28%, beige solid, m.p. 199° C. ¹H NMR (500 MHz, DMSO-d₆) δ 2.90-2.93 (m, 2H, H-2); 3.07-3.10 (m, 2H, H-3); 3.78 (s, 3H, OCH₃); 6.85 (dd, ³=8.6 Hz, ⁴J=2.4 Hz, 1H, H-6); 6.92 (s, 1H, H-4); 7.25 (s, 1H, H-8); 7.62 (d, ³J=8.5 Hz, 1H, H-7); 7.94 (s, 1H, H-10); 9.04 (s, 1H, H-12). IR cm⁻¹: νₘₐₓ 3087, 2924, 2377, 1635, 1548, 1201, 1179, 1073, 919, 864, 825, 801. Anal. (C₁₄H₁₃ONS.HCl) C, H, N.

### 5-[(Z)-(5-Methoxy-2,3-dihydro-1H-indane-1-ylidene)methyl]-1,3-thiazole Hydrochloride (27b)

Prepared from (27i). Purification: FCC (EtOAc:hexane, 1:3). Yield 9%, white solid, m.p. 211° C. ¹H NMR (500 MHz, DMSO-d₆) δ 2.89-2.93 (m, 2H, H-2); 3.07-3.10 (m, 2H, H-3); 3.78 (s, 3H, OCH₃); 6.85 (dd, ³J=8.5 Hz, ⁴J=2.5 Hz, 1H, H-6); 6.92 (s, 1H, H-4); 7.25 (s, 1H, H-8); 7.62 (d, ³J=8.5 Hz, 1H, H-7); 7.93 (s, 1H, H-10); 9.02 (s, 1H, H-12). IR cm⁻¹: νₘₐₓ 2940, 2361, 1598, 1549, 1492, 1318, 1298, 1253, 1110, 1032, 822, 798, 782, 770. Anal. (C₁₄H₁₃ONS.HCl.0.4H₂O) C, H, N.

### 5-[(E)-(5-Fluoro-2,3-dihydro-1H-indane-1-ylidene)methyl]pyrimidine Hydrochloride (28a)

Prepared from (28i). Purification: FCC (EtOAc:hexane, 1:1). Yield 44%, yellow solid, m.p. 212° C. ¹H NMR (500 MHz, DMSO-d₆) δ 3.08-3.15 (m, 4H, H-2, H-3); 6.99 (t, ⁴J=2.5 Hz, 1H, H-8); 7.14 (m, 1H, H-6); 7.21 (dd, ³J=9.1 Hz, ⁴J=2.5 Hz, 1H, H-4); 7.78 (dd, ³J=8.5 Hz, ⁴J=5.4 Hz, 1H, H-7); 8.93 (s, 2H, H-10, H-14); 9.03 (s, 1H, H-12). IR cm⁻¹: νₘₐₓ 3074, 2970, 2322, 1638, 1569, 1528, 1483, 901, 859, 845. Anal. (C₁₄H₁₁N₂F.HCl) C, H, N.

### 5-[(Z)-(5-Fluoro-2,3-dihydro-1H-indane-1-ylidene)methyl]pyrimidine Hydrochloride (28b)

Prepared from (28i). Purification: FCC (EtOAc:hexane, 1:1). Yield 13%, yellow solid, m.p. 204° C. ¹H NMR (500 MHz, DMSO-d₆) δ 2.96 (m, 4H, H-2, H-3); 6.51 (s, 1H, H-8); 6.88 (m, 1H, H-6); 6.98 (dd, ³J=8.8 Hz, ⁴J=5.4 Hz, 1H, H-7); 7.78 (dd, ³J=9.1 Hz, ⁴J=2.2 Hz, 1H, H-4); 8.77 (s, 2H, H-10, H-14); 9.12 (s, 1H, H-12). IR cm⁻¹: νₘₐₓ 3102, 3055, 2955, 2923, 2854, 2244, 1729, 1637, 1586, 1476, 1411, 868, 830, 757, 702. Anal. (C₁₄H₁₁N₂F.HCl) C, H, N.

### 5-[(E)-(5-Fluoro-2,3-dihydro-1H-indane-1-ylidene)methyl]quinoline Hydrochloride (29a)

Purification: FCC (EtOAc:hexane, 1:1). Yield 28%, yellow solid, m.p. 228° C. ¹H NMR (500 MHz, DMSO-d₆) δ 2.82 (m, 4H, H-2, H-3); 6.94 (dt, ³J=9.1 Hz, 43=2.2 Hz, 1H, H-6); 6.99 (dd, ³J=9.1 Hz, ⁴J=2.5 Hz, 1H, H-4); 7.36 (dd, ³J=8.5 Hz, ⁴J=4.1 Hz, 1H, H-15); 7.46 (s, 1H, H-8); 7.54-7.60 (m, 2H, H-7, H-10); 7.73 (d, ³J=8.2 Hz, 1H, H-16); 7.78 (dd, ³J=8.5 Hz, ⁴J=5.6 Hz, 1H, H-11); 8.49 (d, ³J=8.5 Hz, 1H, H-12); 8.72 (m, 1H, H-14). IR cm⁻¹: νₘₐₓ 2925, 2854, 1578, 1356, 1244, 813. Anal. (C₁₉H₁₄NF.HCl.0.5H₂O) C, H, N.

### 5-[(Z)-(5-Fluoro-2,3-dihydro-1H-indane-1-ylidene)methyl]quinoline Hydrochloride (29b)

Purification: FCC (EtOAc:hexane, 1:1). Yield 12%, yellow solid, m.p. 185° C. ¹H NMR (500 MHz, DMSO-d₆) δ 3.17-3.24 (m, 4H, H-2, H-3); 6.53 (dd, ³J=8.5 Hz, ⁴J=5.6 Hz, 1H, H-7); 6.78 (dt, ³J=9.1 Hz, ⁴J=2.5 Hz, 1H, H-6); 7.05 (s, 1H, H-8); 7.28 (dd, ³J=9.1 Hz, ⁴J=2.5 Hz, 1H, H-4); 7.64 (dd, ³J=8.5 Hz, ⁴J=4.1 Hz, 1H, H-15); 7.71 (d, 3J=6.9 Hz, 1H, H-10); 7.93 (t, ³J=7.3 Hz, 1H, H-11); 8.16 (d, J=8.5 Hz, 1H, H-16); 8.52 (d, ³J=9.1 Hz, 1H, H-12); 9.07 (m, 1H, H-14). IR cm⁻¹: νₘₐₓ 2930, 2852, 1563, 1340, 1250, 979, 813. Anal. (C₁₉H₁₄NF.HCl) C, H, N.

### 5-[(E)-(5-Fluoro-2,3-dihydro-1H-indane-1-ylidene)methyl]isoquinoline Hydrochloride (30a)

Purification: FCC (EtOAc:hexane, 1:1). Yield 28%, yellow solid, m.p. 234° C. ¹H NMR (500 MHz, DMSO-d₆) δ 3.03 (s, 4H, H-2, H-3); 7.13 (dt, ³J=9.1 Hz, ⁴J=2.2 Hz, 1H, H-6); 7.17 (dd, ³J=9.1 Hz, ⁴J=2.2 Hz, 1H, H-4); 7.62 (s, 1H, H-8); 7.70 (t, ³J=7.6 Hz, 1H, H-11); 7.91 (d, ³J=7.3 Hz, 1H, H-10); 7.97-8.02 (m, 2H, H-7, H-12); 8.12 (d, ³J=6.0 Hz, 1H, H-16); 8.52 (d, ³J=6.0 Hz, 1H, H-15); 9.31 (s, 1H, H-13). IR cm⁻¹: νₘₐₓ 3045, 2490, 1645, 1602, 1481, 1243, 825. Anal. (C₁₉H₁₄NF.HCl.0.4H₂O) C, H, N.

### 5-[(Z)-(5-Fluoro-2,3-dihydro-1H-indane-1-ylidene)methyl]isoquinoline Hydrochloride (30b)

Purification: FCC (EtOAc:hexane, 1:1). Yield 9%, yellow solid, m.p. 201° C. ¹H NMR (500 MHz, DMSO-d₆) δ 3.09-3.16 (m, 4H, H-2, H-3); 6.48 (dd, ³J=8.5 Hz, ⁴J=5.4 Hz, 1H, H-7); 6.70 (dt, ³J=9.1 Hz, ⁴J=2.5 Hz, 1H, H-6); 6.95 (s, 1H, H-8); 7.21 (dd, ³J=9.1 Hz, 4J=2.5 Hz, 1H, H-4); 7.76-7.83 (m, 2H, H-10, H-11); 7.86 (d, ³J=6.0 Hz, 1H, H-16); 8.18 (d, ³J=8.2 Hz, 1H, H-12); 8.53 (d, ³J=6.0 Hz, 1H, H-15); 9.43 (s, 1H, H-13). IR cm⁻¹: νₘₐₓ 3675, 2969, 2901, 2498, 1730, 1647, 1471, 1065, 818. Anal. (C₁₉H₁₄NF.HCl) C, H, N.

### 4-[(E)-(5-Fluoro-2,3-dihydro-1H-indane-1-ylidene)methyl]quinoline Hydrochloride (31a)

Purification: FCC (EtOAc:hexane, 1:1). Yield 34%, yellow solid, m.p. 241° C. ¹H NMR (500 MHz, DMSO-d₆) δ 3.29-3.38 (m, 4H, H-2, H-3); 7.41 (m, 1H, H-6); 7.46 (d, ³J=9.1 Hz, 1H, H-4); 7.86-7.89 (m, 2H, H-7, H-15); 7.95 (s, 1H, H-8); 8.01 (t, ³J=8.2 Hz, 1H, H-14); 8.26-8.31 (m, 2H, H-10, H-16); 8.61 (d, ³J=8.2 Hz, 1H, H-13); 9.13 (d, 3J=4.4 Hz, 1H, H-11). IR cm⁻¹: νₘₐₓ 2929, 2360, 2341, 1574, 1244, 836, 759. Anal. (C₁₉H₁₄NF.HCl.0.2H₂O) C, H, N.

### 4-[(E)-(5-Fluoro-2,3-dihydro-1H-indane-1-ylidene)methyl]quinoline Hydrochloride (31b)

Purification: FCC (EtOAc:hexane, 1:1). Yield 24%, yellow solid, m.p. 229° C. ¹H NMR (500 MHz, DMSO-d₆) δ 3.27-3.3819 (m, 4H, H-2, H-3); 6.71 (m, 1H, H-7); 6.82 (dt, ³J=9.1 Hz, ⁴J=2.5 Hz, 1H, H-6); 7.06 (s, 1H, H-8); 7.32 (dd, ³J=9.1 Hz, ⁴J=2.5 Hz, 1H, H-4); 7.64 (d, ³J=4.4 Hz, 1H, H-10); 7.72 (t, ³J=7.3 Hz, 1H, H-15); 7.94 (t, ³J=8.5 Hz, 1H, H-14); 8.19 (d, ³J=8.5 Hz, 1H, H-16); 8.23 (d, ³J=8.5 Hz, 1H, H-13); 9.05 (d, ³J=4.4 Hz, 1H, H-11). IR cm⁻¹: νₘₐₓ 2928, 2593, 1581, 1244, 856, 829, 761. Anal. (C₁₉H₁₄NF.HCl.0.3H₂O) C, H, N.

### 3-(9H-Fluorene-9-ylidenemethyl)pyridine Hydrochloride (32)

Purification: FCC (EtOAc:hexane, 1:1). Yield 35%, yellow solid, m.p. 241° C. ¹H NMR (500 MHz, DMSO-d₆) δ 7.14 (m, 1H, arom-H); 7.31 (m, 1H, arom-H); 7.44 (m, 3H, H-6, arom-H); 7.89 (m, 3H, arom-H); 7.99 (m, 2H, arom-H, H-11); 8.58 (d, ³J=7.9 Hz, 1H, H-12); 8.88 (dd, ³J=4.1 Hz, ⁴J=1.2 Hz, 1H, H-10); 9.08 (d, ⁴J=1.9 Hz, 1H, H-8). IR cm⁻¹: νₘₐₓ 3042, 3007, 2955, 2423, 1540, 1442, 809, 767, 722. Anal. (C₁₉H₁₃N.HCl) C, H, N.

### 4-(9H-Fluorene-9-ylidenemethyl)pyridine Hydrochloride (34)

Purification: FCC (EtOAc:hexane, 1:1). Yield 57%, yellow solid, m.p. 258° C. ¹H NMR (500 MHz, DMSO-d₆) δ 7.15 (m, 1H, arom-H); 7.39-7.50 (m, 4H, arom-H, H-6); 7.89 (m, 2H, arom-H); 7.94 (s, 1H, arom-H); 8.00 (m, 2H, arom-H); 8.16 (d, ³J=6.6 Hz, 2H, H-8, H-12); 8.94 (d, ³J=6.6 Hz, 2H, H-10, H-11). IR cm⁻¹: νₘₐₓ 3050, 3004, 2950, 1627, 1585, 1498, 1481, 807, 775, 727. Anal. (C₁₉H₁₃N.HCl.0.3H₂O) C, H, N.

### 3-[(E)-(3-Methyl-2,3-dihydro-1H-indane-1-ylidene)methyl]pyridine Hydrochloride (36a)

Purification: FCC (EtOAc:hexane, 2:3). Yield 36%, yellow solid, m.p. 191.3° C. ¹H NMR (500 MHz, DMSO-d₆) δ 1.28 (d, ³J=6.8 Hz, 3H, CH₃); 2.66 (m, 1H, H-3); 3.34-3.40 (m, 2H, H-2); 7.18 (s, 1H, H-8); 7.30-7.40 (m, 3H, H-4, H-5, H-6); 7.70 (d, ³J=7.8 Hz, 1H, H-7); 7.92 (m, 1H, H-13); 8.48 (d, ³J=8.6 Hz, 1H, H-14); 8.64 (d, 3J=5.4 Hz, 1H, H-12) 8.88 (s, 1H, H-10). IR cm⁻¹: νₘₐₓ 2957, 2538, 1634, 1551, 1474, 762. Anal. (C₁₆H₁₅N.HCl) C, H, N.

### 3-[(Z)-(3-Methyl-2,3-dihydro-1H-indane-1-ylidene)methyl]pyridine Hydrochloride (36b)

Purification: FCC (EtOAc:hexane, 2:3). Yield 14%, white solid, m.p. n.d. ¹H NMR (500 MHz, DMSO-d₆) δ 1.32 (d, ³J=6.8 Hz, 3H, CH₃); 2.48 (m, 1H, H-3); 3.06-3.32 (m, 2H, H-2); 6.60 (s, 1H, H-8); 7.00 (m, 2H, H-5, H-6); 7.16-7.43 (m, 3H, H-4, H-7, H-13); 7.95 (m, 1H, H-14); 8.42 (m, 1H, H-12); 8.74-8.95 (m, 1H, H-10). IR cm⁻¹: νₘₐₓ 2955, 2865, 2424, 1610, 1546, 1454, 818, 765. Anal. (C₁₆H₁₅N—HCl) C, H, N.

### 3-[(E)-(3-Phenyl-2,3-dihydro-1H-indane-1-ylidene)methyl]pyridine Hydrochloride (37a)

Purification: FCC (EtOAc:hexane, 2:8). Yield 140%, yellow solid, m.p. 188° C. ¹H NMR (500 MHz, DMSO-d₆) δ 3.05-3.10 (m, 1H, H-2); 3.63-3.68 (m, 1H, H-2); 4.57-4.59 (m, 1H, H-3); 7.00 (t, ⁴J=2.4 Hz, 1H, H-8); 7.10-7.40 (m, 8H, H-4, H-5, H-6, Phenyl); 7.57 (dd, 3J=8.1 Hz, 3J=5.1 Hz, 1H, H-13); 7.71 (d, ³J=7.3 Hz, 1H, H-7); 8.07 (d, ³J=8.3 Hz, 1H, H-14); 8.46 (d, ³J=6.3 Hz, 1H, H-12); 8.74 (s, 1H, H-10). IR cm⁻¹: νₘₐₓ 3024, 2863, 2471, 1639, 1542, 811, 766, 757. Anal. (C₂₁H₁₇N.HCl) H, N, C: calc. 78.86. found 80.00.

### 3-[(1E)-1-(2,3-Dihydro-1H-indane-1-ylidene)ethyl]pyridine Hydrochloride (38a)

Purification: FCC (EtOAc:hexane, 1:1). Yield 60%, yellow solid, m.p. 187° C. ¹H NMR (500 MHz, DMSO-d₆) δ 2.33 (t, ⁴J=1.9 Hz, 3H, CH₃); 2.63-2.84 (m, 4H, H-2, H-3); 7.10 (dt, ³J=9.5 Hz, ⁴J=2.5 Hz, 1H, H-4); 7.17 (dd, ³J=9.1 Hz, ⁴J=2.5 Hz, 1H, H-6); 7.41 (m, 1H, H-13); 7.70-7.76 (m, 2H, H-7, H-14); 8.46 (dd, ³J=5.0 Hz, ⁴J=1.6 Hz, 1H, H-12); 8.55 (dd, ⁴J=2.5 Hz, ⁴J=0.6 Hz, 1H, H-10). IR cm⁻¹: νₘₐₓ 3029, 2961, 2925, 2360, 1730, 1547, 1475, 1250, 1084, 1019, 933, 859, 825, 816. Anal. (C₁₆H₁₅N.HCl.0.6H₂O) C, H, N.

### Example 2

### Synthesis of imidazolylmethylene-tetrahydronaphthalenes und-indanes

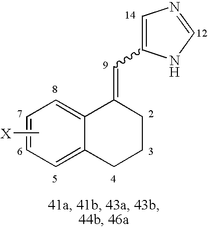

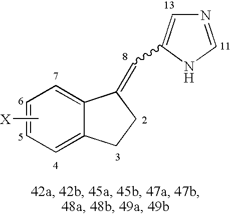

| No. | X | Isomer |
|---|---|---|
| 41a | H | E |
| 41b | H | Z |
| 42a | H | E |
| 42b | H | Z |
| 43a | 7-CN | E |
| 43b | 7-CN | Z |
| 44b | 6-CN | Z |
| 45a | 5-CN | E |
| 45b | 5-CN | Z |
| 46a | 7-Cl | E |
| 47a | 5-F | E |
| 47b | 5-F | Z |
| 48a | 5-Cl | E |
| 48b | 5-Cl | Z |
| 49a | 5-Br | E |
| 49b | 5-Br | Z |

The general synthesis was effected according to the following synthetic scheme:

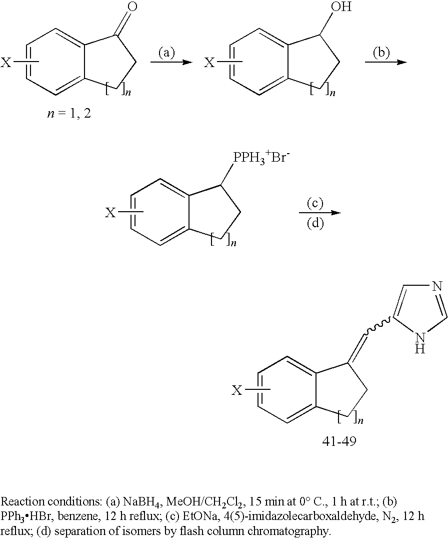

A) Synthesis of the Commercially Unavailable Precursors: The Following Compounds were Prepared by Known Synthetic Methods:

7-Hydroxy-1-tetralone (43iii), 6-hydroxytetralone (44iii), 5-hydroxyindane-1-one (45iii) (all according to: Woo, L. W. et al. J. Med. Chem. 41: 1068-1083 (1998)), 8-oxo-5,6,7,8-tetrahydronaphth-2-yl-trifluoromethylsulfonate (431i) (Almansa, C. et al., Synth. Commun. 23: 2965-2971 (1993); Gerlach, U. & Wollmann, T., Tetrahedron Letters 33: 5499-5502 (1992)), 5-oxo-5,6,7,8-tetrahydronaphth-2-yl-trifluoromethylsulfonate (44ii), 1-oxo-2,3-dihydro-1H-indene-5-yl-trifluoromethylsulfonate (45ii) (Almansa, C. et al., Synth. Commun. 23: 2965-2971 (1993)), 8-oxo-5,6,7,8-tetrahydronaphthalene-2-carbonitrile (43i), 5-oxo-5,6,7,8-tetrahydronaphthalene-2-carbonitrile (44i) (Almansa, C. et al., Synth. Commun. 23: 2965-2971 (1993)), 1-oxoindane-5-carbonitrile (45i) (Almansa, C. et al., Synth. Commun. 23: 2965-2971 (1993); Arnold, D. R. et al., Can. J. Chem. 73: 307-318 (1995)), 7-chloro-3,4-dihydro-2H-naphth-1-one (46i) (Kerr, C. A. & Rae, I. D., Aust. J. Chem. 31: 341-346 (1978); Skraup, S. & Schwamberger, E., Liebigs Ann. Chem. 462: 135-158 (1928); Martin, E. L., J. Am. Chem. Soc. 58: 1438-1442 (1936); Koo, J., J. Am. Chem. Soc. 75: 1891-1895 (1953)).

The synthesis was effected according to the following reaction schemes:

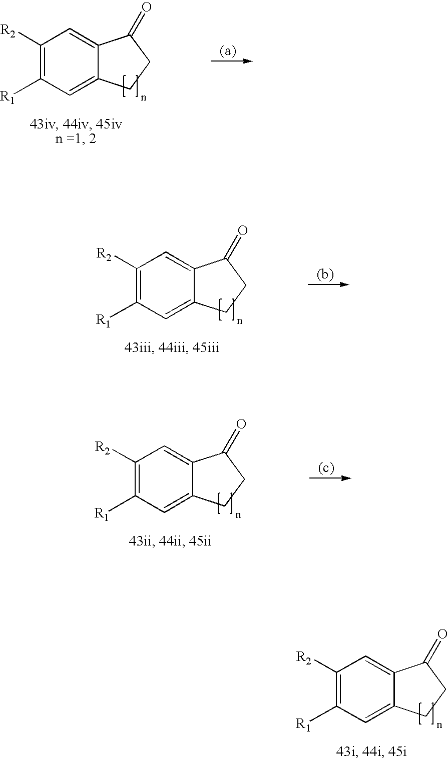

| | R₁ | R₂ | n |
|---|---|---|---|
| 43iv | H | OMe | 2 |
| 44iv | OMe | H | 2 |
| 45iv | OMe | H | 1 |
| 43iii | H | OH | 2 |
| 44iii | OH | H | 2 |
| 45iii | OH | H | 1 |
| 43ii | H | OTf | 2 |
| 44ii | OTf | H | 2 |
| 45ii | OTf | H | 1 |
| 43i | H | CN | 2 |
| 44i | CN | H | 2 |
| 45i | CN | H | 1 |

Reaction conditions:
(a) AlCl₃, benzene, 3 h reflux;
(b) trifluoromethanesulfonic anhydride, dry pyridine, 15 min at 0-5° C., then 2 h at r.t.;
(c) Zn, PPh₃, KCN, Ni(PPh₃)₂Cl₂, MeCN, 2 h at 60° C.

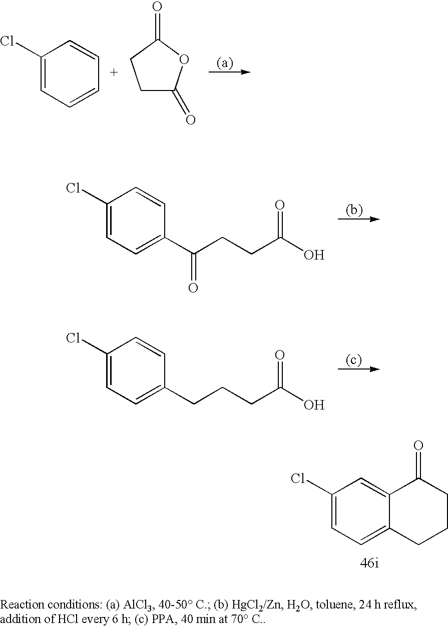

### Purification Conditions, Yield and Characterization of Precursors:

1-Oxo-2,3-dihydro-1H-indene-5-yl-trifluoromethylsulfonate (451i). Purification: bulb tube distillation. Yield 82%, yellow oil. ¹H NMR (400 MHz, CDCl₃) δ 2.50-2.81 (m, 2H, H-2); 3.00-3.40 (m, 2H, H-3); 7.20-7.50 (m, 2H, H-4, H-6); 7.95 (d, ³J=8.5 Hz, 1H, H-7). IR (NaCl) cm⁻¹: νₘₐₓ 3060, 2920, 1720, 1610, 1590, 1210, 1140, 1090, 930.

8-Oxo-5,6,7,8-tetrahydronaphthalene-2-carbonitrile (43i). Purification: Crystallization from ligroin. Yield 670%, yellow crystals, m.p. 154° C. ¹H NMR (400 MHz, CDCl₃) δ 2.16-2.22 (m, 2H, H-3); 2.71 (t, ³J=6.2 Hz, 2H, H-4); 3.05 (t, ³J=6.2 Hz, 2H, H-2); 7.42 (d, ³J=8.0 Hz, 1H, H-5); 7.71 (dd, ³J=8.0 Hz, ⁴J=1.8 Hz, 1H, H-6); 8.29 (d, ⁴J=1.8 Hz, 1H, H-8). IR (KBr) cm⁻¹: νₘₐₓ 3060, 3020, 2220, 1680, 1600, 1490, 1430, 1420, 1320, 1280, 1170, 1140, 1030, 920, 830.

B) General synthetic method for compounds 41-49: In a mixture of methanol (110 ml) and dichloromethane (55 ml), 50 mmol of the ketone was dissolved. 1.89 g of NaBH₄ (50 mmol) was added in small portions, cooling the reaction mixture to 0° C. After 15 min at 0° C., the mixture was stirred at room temperature for 1 h, then diluted with water and extracted with diethyl ether. The organic phase was washed first with 1 N HCl, then with a saturated NaHCO₃ solution and finally with water. It was dried over MgSO₄, followed by removing the solvent in vacuo.

The alcohol obtained was converted to the phosphonium salt. Thus, 40 mmol of the alcohol and 13.7 g of triphenylphosphonium bromide (40 mmol) was suspended in 25 ml of benzene and refluxed for 12 h under nitrogen. The precipitate was filtered off and dried, then taken up in dry diethyl ether and stirred for 10 min. The phosphonium salt was finally filtered off and washed with acetone.

A sodium ethanolate solution was prepared by dissolving 0.5 g of sodium (22 mmol) in 20 ml of ethanol, and 2.1 g of imidazole-4(5)-carbaldehyde (22 mmol) was added. The solution was heated slightly for some minutes until it became clear. In a second flask, 20 mmol of phosphonium salt was suspended in 14 ml of ethanol and refluxed under nitrogen atmosphere. The imidazolide solution was added in small portions through a septum within 2 h, and the reaction mixture was refluxed for another 12 h. After cooling down to room temperature, the solid was filtered off and discarded. The filtrate was concentrated, the residue was taken up in 100 ml of water and repeatedly extracted with diethyl ether. The combined organic phases were filtered off over Celite, dried over MgSO₄ and concentrated in vacuo. After being purified with chromatographic methods, the free base was either dissolved in acetone and admixed with an excess of oxalic acid in acetone to obtain the oxalate, or it was dissolved in dry diethyl ether and admixed with an excess of HCl in diethyl ether to obtain the hydrochloride.

### C) Purification Conditions, Yield and Characterization of the Title Compounds:

5-[(E)-3,4-Dihydronaphth-1(2H)-ylidenemethyl]-1H-imidazole (41a). Purification: FCC (acetone) and recrystallization (acetone); yield 14%, colorless crystals, m.p. 136-138° C. ¹H NMR (400 MHz, DMSO-d₆, free base) δ 1.72-1.82 (m, 2H, H-3); 2.68-2.73 (m, 2H, H-2); 2.77-2.82 (m, 2H, H-4); 6.94 (s, 1H, H-9); 7.10-7.22 (m, 4H, H-5, H-7, H-14); 7.66 (d, ³J=7.8 Hz, 1H, H-8); 7.69 (s, 1H, H-12). IR (KBr, free base) cm⁻¹: νₘₐₓ 3110, 3055, 3020, 2935, 2865, 2830, 1666, 1621, 1597, 1481, 1453, 1441, 1430, 951, 943, 765. Anal. (C₁₄H₁₄N₂, free base) C, H, N.

5-[(Z)-3,4-Dihydronaphth-1(2H)-ylidenemethyl]-1H-imidazolium oxalate (41b). Purification: FCC (CHCl₃:DMF, 9:2). yield 50%, white solid, m.p. 187° C. ¹H NMR (400 MHz, DMSO-d₆) δ 1.87-1.94 (m, 2H, H-3); 2.46-2.50 (m, 2H, H-2); 2.81-2.84 (m, 2H, H-4); 6.24 (s, 1H, H-9); 7.00-7.20 (m, 4H, H-5, H-6, H-7, H-14); 7.37 (d, ³J=7.8 Hz, 1H, H-8); 8.31 (s, 1H, H-12). IR (KBr) cm⁻¹: νₘₐₓ 1610, 1480, 1450, 920, 850, 760. Anal. (C₁₄H₁₄N₂.C₂H₂O₄) C, H, N.

5-[(E)-2,3-Dihydro-1H-indane-1-ylidenemethyl]-1H-imidazole (42a). Purification: FCC (acetone); yield 28%, white solid, m.p. 148-151° C. ¹H NMR (400 MHz, DMSO-d₆, free base) δ 2.89-2.96 (m, 2H, H-2); 3.00-3.08 (m, 2H, H-3); 6.92 (t, ⁴J=2.5 Hz, 1H, H-8); 7.13-7.25 (m, 3H, H-5, H-6, H-13); 7.30 (d, ³J=6.5 Hz, 1H, H-4); 7.57 (d, ³J=6.8 Hz, 1H, H-7); 7.69 (s, 1H, H-11). IR (KBr, free base) cm⁻¹: νₘₐₓ 3055, 3020, 2960, 2920, 2840, 1643, 1601, 1460, 985, 757. Anal. (C₁₃H₁₂N₂.C₂H₂O₄) C, H, N.

5-[(Z)-2,3-Dihydro-1H-ind-1-ylidenemethyl]-1H-imidazolium oxalate (42b). Purification: FCC (CHCl₃:DMF, 9:2); yield 100%, white solid, m.p. 196° C. ¹H NMR (400 MHz, DMSO-d₆) δ 2.85-2.94 (m, 4H, H-2, H-3); 6.36 (s, 1H, H-8); 7.14 (t, ³J=7.4 Hz, 1H, H-5); 7.24 (t, ³J=7.4 Hz, 1H, H-6); 7.31 (d, ³J=7.4 Hz, 1H, H-4); 7.37 (s, 1H, H-13); 8.13 (d, ³J=7.4 Hz, 1H, H-7); 8.30 (s, 1H, imidazole-H-11). IR (KBr) cm⁻¹: νₘₐₓ 1610, 1450, 750, 720. Anal. (C₁₃H₁₂N₂.0.75 C₂H₂O₄) C, H, N.

(8E)-8-(1H-Imidazole-5-ylmethylene)-5,6,7,8-tetrahydronaphthalene-2-carbonitrile hydrochloride (43a). From compound 43i. Purification: FCC (CHCl₃:DMF, 9:2); yield 11%, green crystals, m.p. 217° C. ¹H NMR (400 MHz, DMSO-d₆) δ 1.80-1.84 (m, 2H, H-2); 2.71-2.74 (m, 2H, H-2); 2.81-2.84 (m, 2H, H-4); 7.10 (s, 1H, H-9); 7.42 (d, 3J=7.9 Hz, 1H, H-5); 7.68 (d, ³J=7.9 Hz, 1H, H-6); 7.82 (s, 1H, H-14); 8.13 (s, 1H, H-8); 9.11 (s, 1H, H-12). IR (KBr, free base) cm⁻¹: νₘₐₓ 2940, 2220, 1620, 1600, 1490, 1000, 900, 880, 840, 820. Anal. (C₁₅H₁₃N₃, free base) C, H, N.

(8Z)-8-(1H-Imidazole-5-ylmethylene)-5,6,7,8-tetrahydronaphthalene-2-carbonitrile oxalate (43b). From compound 43i. Purification: FCC (CHCl₃:DMF, 9:2); yield 30%, white solid, m.p. 197° C. ¹H NMR (400 MHz, DMSO-d₆) δ 1.90-1.92 (m, 2H, H-3); 2.47-2.51 (m, 2H, H-2); 2.88-2.90 (m, 2H, H-4); 6.37 (s, 1H, H-9); 7.19 (s, 1H, H-14); 7.36 (d, ³J=8.0 Hz, 1H, H-5); 7.58 (dd; ³J=8.0 Hz, ⁴J=1.6 Hz, 1H, H-6); 7.95 (s, 1H, H-8); 8.13 (s, 1H, H-12). IR (KBr) cm⁻¹: νₘₐₓ 2840, 2240, 1600, 1600, 1000, 940, 930, 850, 820, 710. Anal. (C₁₅H₁₃N₃.C₂H₂O₄) C, H, N.

(5Z)-5-(1H-Imidazole-5-ylmethylene)-5,6,7,8-tetrahydronaphthalene-2-carbonitrile oxalate (44b). From compound 44i. Purification: FCC (CHCl₃:DMF, 9:2); yield 30%, white solid, m.p. 206° C. ¹H NMR (400 MHz, DMSO-d₆) δ 1.86-1.93 (m, 2H, H-3); 2.47-2.50 (m, 2H, H-2); 2.83-2.86 (m, 2H, H-3); 6.41 (s, 1H, H-9); 7.18 (s, 1H, H-14); 7.45 (dd, ³J=8.1 Hz, ⁴J=1.6 Hz, 1H, H-7); 7.66-7.68 (m; 2H, arom-H5, H-8); 8.12 (s, 1H, H-12). IR (KBr) cm⁻¹: νₘₐₓ 2220, 1610, 850, 710. Anal. (C₁₅H₁₃N₃.C₂H₂O₄) C, H, N.

(1E)-1-(1H-Imidazole-5-ylmethylene)indane-5-carbonitrile oxalate (45a). From compound 45i. Purification: FCC (CHCl₃:DMF, 9:2); yield 390%, white solid, m.p. 217° C. ¹H NMR (400 MHz, DMSO-d₆) δ 2.96-2.99 (m, 2H, H-2); 3.14-3.17 (m, 2H, H-3); 7.16 (s, 1H, H-8); 7.72-7.79 (m, 3H, H-4, H-6, H-7); 7.83 (s, 1H, H-13); 9.17 (s, 1H, H-11). IR (KBr) cm⁻¹: νₘₐₓ 3080, 2220, 1600, 830. Anal. (C₁₄H₁₂N₃.HCl) C, H, N: calc. 16.31. found 15.71.

(1Z)-1-(1H-Imidazole-5-ylmethylene)indane-5-carbonitrile oxalate (45b). From compound 45i. Purification: FCC (CHCl₃:DMF, 9:2); yield 100%, white solid, m.p. 207° C. ¹H NMR (400 MHz, DMSO-d₆) δ 2.89-2.99 (m, 4H, H-2, H-3); 6.57 (s, 1H, H-8); 7.36 (s, 1H, H-4); 7.60 (d, ³J=8.2 Hz, 1H, H-6); 7.72 (s, 1H, H-13); 8.02 (s, 1H, H-11); 9.13 (d; ³J=8.2 Hz, 1H, H-7). IR (KBr) cm⁻¹: νₘₐₓ 2220, 1620, 890, 830, 780, 720. Anal. (C₁₄H₁₂N₃.0.8C₂H₂O₄) C, H, N.

5-[(E)-(6-Chloro-3,4-dihydronaphth-1(2H)-ylidene)methyl]-1H-imidazolium chloride (46a). From compound 46i. Purification: FCC (CHCl₃:DMF, 9:2); yield 35%, nacreous crystals, m.p. 203° C. ¹H NMR (400 MHz, DMSO-d₆, free base) δ 1.73-1.80 (m, 2H, H-3); 2.67-2.70 (m, 2H, H-2); 2.80-2.84 (m, 2H, H-4); 6.99 (s, 1H, H-9); 7.16-7.25 (m, 3H, H-5, H-6, H-14); 7.67 (s, 1H, H-8); 7.76 (s, 1H, H-12). IR (KBr, free base) cm⁻¹: νₘₐₓ 3060, 2940, 2840, 1590, 1120, 950, 870, 850, 840, 800. Anal. (C₁₄H₁₃ClN₂.0.2H₂O) C, H, N.

5-[(E)-(5-Fluoro-2,3-dihydro-1H-indene-1-ylidenemethyl]-1H-imidazolium chloride (47a). Purification: FCC (CHCl₃:DMF, 9:2); yield 49%, beige crystals, m.p. 245° C. ¹H NMR (400 MHz, DMSO-d₆) δ 2.96-3.01 (m, 2H, H-2); 3.10-3.13 (m, 2H, H-3); 6.91 (s, 1H, H-8); 7.11-7.16 (m, 1H, H-6); 7.21 (d, ³J(H,F)=9.0 Hz, 1H, H-4); 7.64 (dd, ³J=8.5 Hz, ⁴H(H,F)=5.3 Hz, 1H, H-7); 7.69 (s, 1H, H-13); 9.14 (s, 1H, H-11). IR (KBr) cm⁻¹: νₘₐₓ 3080, 1600, 1590, 1090, 940, 870. Anal. (C₁₃H₁₁FN₂.HCl) C, H, N.

5-[(Z)-(5-Fluoro-2,3-dihydro-1H-indene-1-ylidene)methyl]-1H-imidazolium oxalate (47b). Purification: FCC (CHCl₃:DMF, 9:2); yield 15%, white solid, m.p. 205° C. ¹H NMR (400 MHz, DMSO-d₆) δ 2.88-2.93 (m, 4H, H-2, H-3); 6.33 (s, 1H, H-8); 6.98 (m, 1H, H-6); 7.14 (d, ³J(H,F)=9.1 Hz, 1H, H-4); 7.39 (s, 1H, H-13); 8.27-8.31 (m, 2H, H-7, H-11). IR (KBr) cm⁻¹: νₘₐₓ 1600, 1480, 1220, 930, 860, 830, 720. Anal. (C₁₃H₁₁FN₂.C₂H₂O₄) H, N, C: calc. 59.21. found 59.70.

5-[(E)-(5-Chloro-2,3-dihydro-1H-indene-1-ylidene)methyl]-1H-imidazolium chloride (48a). Purification: FCC (CHCl₃:DMF, 9:2); yield 37%, white solid, m.p. 238° C. ¹H NMR (400 MHz, DMSO-d₆) δ 2.95-2.97 (m, 2H, H-2); 3.10-3.13 (m, 2H, H-3>; 6.94 (s, 1H, H-8); 7.34 (d, J=8.3 Hz, 1H, H-6); 7.46 (s, 1H, H-4); 7.64 (d, ³J=8.3 Hz, 1H, H-7); 7.72 (s, 1H, H-13); 9.11 (s, 1H, H-11). IR (KBr) cm⁻¹: νₘₐₓ 3080, 1600, 1470, 1290, 1200, 1110, 1070, 830, 610. Anal. (C₁₃H₁₁ClN₂.HCl) C, H, N.

5-[(Z)-(5-Chloro-2,3-dihydro-1H-indene-1-ylidene)methyl]-1H-imidazolium oxalate (48b). Purification: FCC (CHCl₃:DMF, 9:2); yield 170%, white solid, m.p. 218° C. ¹H NMR (400 MHz, DMSO-d₆) δ 2.87-2.96 (m, 4H, H-2, H-3); 6.39 (s, 1H, H-8); 7.20 (dd, ³J=8.4 Hz, ⁴J=2.0 Hz, 1H, H-6); 7.37-7.38 (m, 2H, H-4, H-13); 8.24 (s, 1H, H-11); 8.50 (d, ³J=8.4 Hz, 1H, H-7). IR (KBr) cm⁻¹: νₘₐₓ 1600, 1470, 1210, 880, 860, 830, 710. Anal. (C₁₃H₁₁ClN₂.C₂H₂O₄) C, H, N.

5-[(E)-(5-Bromo-2,3-dihydro-1H-indene-1-ylidene)methyl]-1H-imidazolium chloride (49a). Purification: FCC (CHCl₃:DMF, 9:2); yield 470%, white solid, m.p. 228° C. ¹H NMR (400 MHz, DMSO-d₆) δ 2.42-2.61 (m, 2H, H-2); 3.10-3.12 (m, 2H, H-3); 7.00 (s, 1H, H-8); 7.47 (d, ³J=8.3 Hz, 1H, H-6); 7.54-7.59 (m, 2H, H-4, H-7); 7.71 (s, 1H, H-13); 9.15 (s, 1H, H-11). IR (KBr) cm⁻¹: νₘₐₓ 3080, 1600, 1590, 1470, 830. Anal. (C₁₃H₁₁BrN₂.HCl) H, N, C: calc. 50.11. found 49.69.

5-[(Z)-(5-Bromo-2,3-dihydro-1H-indene-1-ylidene)methyl]-1H-imidazolium oxalate (49b). Purification: FCC (CHCl₃:DMF, 9:2); yield 110%, white solid, m.p. 218° C. ¹H NMR (400 MHz, DMSO-d₆) δ 2.87-2.95 (m, 4H, H-2, H-3); 6.40 (s, 1H, H-8); 7.32-7.35 (m, 2H, H-6, H-13); 7.50 (s, 1H, H-4); 8.12 (s, 1H, H-11); 8.53 (d, ³J=8.4 Hz, 1H, H-7). IR (KBr) cm⁻¹: νₘₐₓ 1620, 1460, 1400, 1100, 1070, 820, 780, 720. Anal. (C₁₃H₁₁BrN₂.0.8C₂H₂O₄) C, H, N.

### Example 3

### Alternative Preparation Method for Imidazole Compounds

A) Comparative synthesis: Synthesis of imidazolyl-substituted indanes according to Mitrenga (Mitrenga, M., Dissertation Universität Saarbrücken 1996, Shaker-Verlag, Aachen, Germany (1997))

The synthesis was effected as described under Ex. 2B (“general synthesis”) according to the reaction scheme:

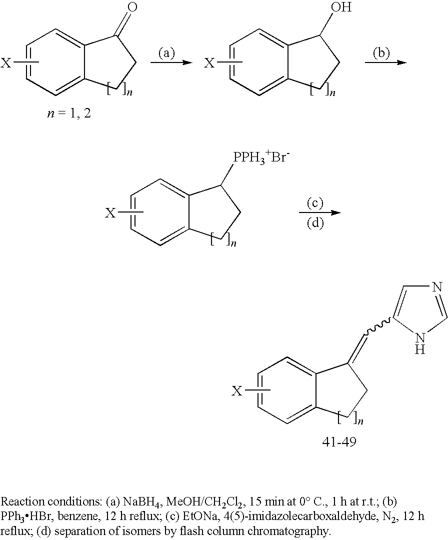

However, firstly, this synthesis was suitable only for the preparation of imidazolyl compounds since the imidazolyl aldehyde employed was employed as the base and at the same time as a reactant. The yields for the Z isomer were always smaller than 20%, in most cases even smaller than 10%.

Secondly, this synthesis was surprisingly not suitable for the routine preparation of 42a/b and 48a/b: in comparative experiments, no product could be isolated. The difficulty of this reaction presumably resides in the preparation of the imidazolyl anion by NaOEt. This anion does not seem to be particularly stable. Therefore, for performing the reaction in an always successful way, very dry conditions under protective gas would be necessary, since the anion decays already when transferred semi-inertly from one flask to another.

Moreover, the yields according to the preparation method shown under B) were usually clearly higher as compared to the general method.

### B) Alternative Preparation Method for Compounds (42a, 42b, 48a, 48b) as Hydrochlorides

The alternative synthesis was effected according to the synthetic scheme

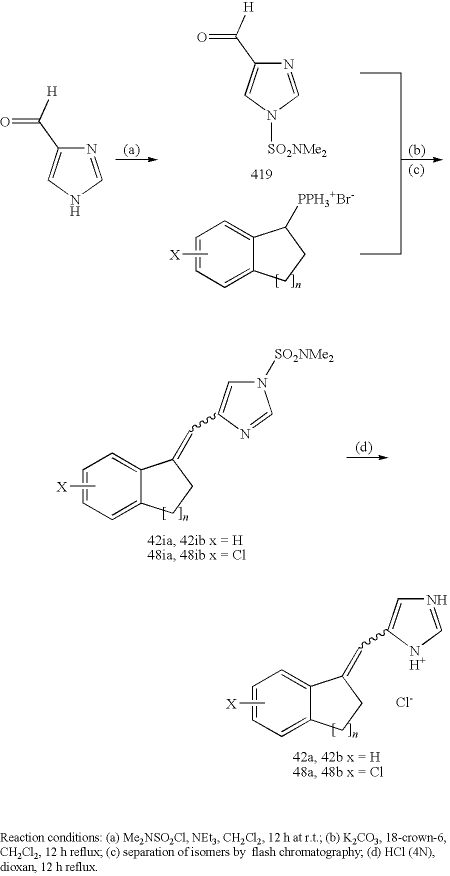

The reaction has the advantage that the imidazole substituent is protected, and the separation of the isomers by flash chromatography can be effected more easily than with unprotected imidazoles (which “smear” on the column, and more complicated mixtures of mobile solvents are required to separate the products neatly). The separation of the protective groups is effected without problems and quantitatively by HCl, and the desired salt directly precipitates at the end of the reaction. By this method, the yield could be enhanced clearly as compared to the general method.

Performance: 4-Formyl-N,N-dimethyl-1H-imidazolyl-1-sulfonamide (419) was prepared as described (Kim, J. et al., J. Heterocycl. Chem. 32: 611-620 (1995); Chadwick, D. J. & Ngochindo, R. I., J. Chem. Soc. Perkin Trans. I 3: 481-486 (1984)). A suspension of 5 mmol of phosphonium salt (see general synthetic method), 5 mmol of the corresponding alcohol, 50 mmol of K₂CO₃ and 150-200 mg of 18-crown-6 in 25 ml of dry dichloromethane was refluxed for 12 h under nitrogen. The reaction mixture was then poured into water and repeatedly extracted with dichloromethane. The combined organic phases were dried over MgSO₄, and the solvent was removed in vacuo. After having been purified, 2.5 mmol of the sulfonamide (42ia/42ib, 48ia/481b) was taken up in some ml of dioxan, and 75 ml of 4 N HCl was added. The mixture was refluxed with stirring over night. Upon cooling to room temperature, the hydrochloride precipitated and could be filtered off and washed with dry diethyl ether (quantitative yield, based on the sulfonic acid amide).

C) Purification Conditions, Yield and Characterization of the Title Compounds when Prepared According to the Alternative Method B):

5-[(E)-2,3-Dihydro-1H-indene-1-ylidenemethyl]-1H-imidazolyl-1-sulfonic acid dimethylamide (421a). Purification: FCC (EtOAc:hexane, 3:2). Yield 43%, yellow solid, m.p. 122-123° C. ¹H NMR (500 MHz, DMSO-d₆) δ 2.92 (s, 6H, H-methyl); 3.12 (m, 4H, H-2, H-3); 6.97 (s, 1H, H-8); 7.28-7.32 (m, 2H, H-4, H-6); 7.38-7.40 (m, 1H, H-5); 7.61 (s, 1H, H-13); 7.70-7.73 (m, 1H, H-7); 8.28 (d, ⁴J=1.3 Hz, 1H, H-11). IR (powder) cm⁻¹: νₘₐₓ 3122, 2929, 2360, 1684, 1469, 1384, 1169, 1082, 724. Anal. (C₁₅H₁₇N₃SO₂) C, H, N.

5-[(Z)-2,3-Dihydro-1H-indene-1-ylidenemethyl]-1H-imidazolyl-1-sulfonic acid dimethylamide (42ib). Purification: FCC (EtOAc:hexane, 3:2). Yield 16%, yellow solid, m.p. 81° C. ¹H NMR (500 MHz, DMSO-d₆) δ 2.82 (s, 6H, H-methyl); 2.83-2.93 (m, 4H, H-2, H-3); 6.37 (s, 1H, H-8); 7.14-7.22 (m, 2H, H-4, H-6); 7.26 (m, 1H, H-5); 7.61 (s, 1H, H-13); 8.25 (s, 1H, H-11); 8.82 (d, J=7.6 Hz, 1H, H-7). IR (powder) cm⁻¹: νₘₐₓ 3122, 2929, 2361, 1461, 1388, 1176, 1081, 962, 725. Anal. (C₁₅H₁₇N₃SO₂) H, N, C: calc. 59.38. found 60.06.

5-[(E)-(5-Chloro-2,3-dihydro-1H-indene-1-ylidene)methyl]-1H-imidazolyl-1-sulfonic acid dimethylamide (48ia). Purification: FCC (EtOAc:hexane, 3:2). Yield 47%, yellow solid, m.p. 159° C. ¹H NMR (500 MHz, DMSO-d₆) δ 2.84 (s, 6H, H-methyl); 3.06 (m, 4H, H-2, H-3); 6.92 (s, 1H, H-8); 7.27 (dd, ³J=8.2 Hz, ⁴J=1.9 Hz, 1H, H-6); 7.39 (d, ⁴J=1.9 Hz, 1H, H-4); 7.56 (s, 1H, H-13); 7.66 (d, ³J=8.5 Hz, 1H, H-7); 8.22 (s, 1H, H-11). IR (powder) cm⁻¹: νₘₐₓ 3133, 2939, 2360, 1467, 1379, 1165, 1088, 726. Anal. (C₁₅H₁₆N₃SO₂Cl) C, H, N.

5-[(Z)-(5-Chloro-2,3-dihydro-1H-indene-1-ylidene)methyl]-1H-imidazolyl-1-sulfonic acid dimethylamide (48ib). Purification: FCC (EtOAc:hexane, 3:2). Yield 20%, yellow solid, m.p. 135° C. ¹H NMR (500 MHz, DMSO-d₆) δ 2.83 (s, 6H, H-methyl); 2.88-2.97 (m, 4H, H-2, H-3); 6.42 (s, 1H, H-8); 7.24 (dd, ³J=8.5 Hz, ⁴J=1.9 Hz, 1H, H-6); 7.36 (d, ⁴J=1.9 Hz, 1H, H-4); 7.66 (s, 1H, H-13); 8.28 (d, ⁴J=1.3 Hz, 1H, H-11); 9.00 (d, ³J=8.5 Hz, 1H, H-7). IR (powder) cm⁻¹: νₘₐₓ 3124, 2923, 2361, 1465, 1386, 1174, 1080, 962, 724. Anal. (C₁₅H₁₆N₃SO₂Cl) C, H, N.

5-[(E)-2,3-Dihydro-1H-indane-1-ylidenemethyl]-1H-imidazole hydrochloride (42a as hydrochloride). Prepared from 5-[(E)-2,3-dihydro-1H-indane-1-ylidenemethyl]-1H-imidazole-1-sulfonic acid dimethylamide (42ai). Purification: Precipitation of the salt with 4 N HCl, washing the filtrate with dry diethyl ether. Yield: quantitative, based on (42ai). Beige needles, m.p. 247° C.; ¹H NMR (500 MHz, DMSO-d₆) δ 2.99-3.02 (m, 2H, H-2); 3.17-3.20 (m, 2H, H-3); 6.97 (s, 1H, H-8); 7.35-7.40 (m, 2H, H-5, H-6); 7.46 (d, ³J=6.6 Hz, 1H, H-7); 7.70 (d, ³J=7.9 Hz, 1H, H-4); 7.78 (s, 1H, H-13); 9.17 (s, 1H, H-11); 14.60 (s, 2H, NH). IR cm⁻¹: νₘₐₓ 3381, 3165, 3082, 2989, 2822, 2362, 2686, 2651, 1519, 1267, 1142, 841, 815, 748. Anal. (C₁₃H₁₂N₂.HCl.0.5H₂O) C, H, N.

5-[(Z)-2,3-Dihydro-1H-indane-1-ylidenemethyl]-1H-imidazole hydrochloride (42b as hydrochloride). Prepared from 5-[(Z)-2,3-dihydro-1H-indane-1-ylidenemethyl]-1H-imidazole-1-sulfonic acid dimethylamide (42bi). Purification: Precipitation of the salt with 4 N HCl, washing the filtrate with dry diethyl ether. Yield: quantitative, based on (42bi); white solid, m.p. 244° C.; ¹H NMR (500 MHz, DMSO-d₆) δ 3.10-3.13 (m, 2H, H-2); 3.29-3.32 (m, 2H, H-3); 7.09 (t, ⁴J=2.5 Hz, 1H, H-8); 7.47-7.52 (m, 2H, H-5, H-6); 7.56-7.58 (m, 1H, H-7); 7.81-7.83 (m, 1H, H-4); 7.90 (s, 1H, H-13); 9.28 (s, 1H, H-11); 14.75 (s, 2H, NH). IR (KBr) cm⁻¹: νₘₐₓ 3080, 2818, 1651, 1605, 1479, 1178, 1097, 840. Anal. (C₁₃H₁₂N₂.HCl.HCl) C, H, N: calc., 11.17. found, 11.95.

5-[(E)-(5-Chloro-2,3-dihydro-1H-indene-1-ylidene)methyl]-1H-imidazole hydrochloride (48a as hydrochloride). Prepared from 5-[(E)-2,3-dihydro-1H-indane-1-ylidenemethyl]-1H-imidazole-1-sulfonic acid dimethylamide (48ai). Purification: Precipitation of the salt with 4 N HCl, washing the filtrate with dry diethyl ether. Yield: quantitative, based on (48ai). Yellow solid, m.p. 245° C.; ¹H NMR (500 MHz, DMSO-d₆) δ 3.01-3.05 (m, 2H, H-2); 3.10-3.13 (m, 2H, H-3); 6.96 (t, ⁴J=2.5 Hz, 1H, H-8); 7.43 (dd, ³J=8.2 Hz, ⁴J=1.9 Hz, 1H, H-6); 7.54 (d, ⁴J=1.6 Hz, 1H, H-4); 7.74 (d, ³J=8.2 Hz, 1H, H-7); 7.79 (s, 1H, H-13); 9.13 (s, 1H, H-11). IR cm⁻¹: νₘₐₓ 3080, 1600, 1470, 1290, 1200, 1110, 1070, 830, 610. IR (KBr) cm⁻¹: νₘₐₓ 3087, 2998, 2931, 2772, 2615, 1603, 1467, 1069, 831, 816. Anal. (C₁₃H₁₁ClN₂.HCl) C, H, N.

5-[(Z)-(5-Chloro-2,3-dihydro-1H-indane-1-ylidene)methyl]-1H-imidazole hydrochloride (48b as hydrochloride). Prepared from 5-[(Z)-2,3-dihydro-1H-indane-1-ylidenemethyl]-1H-imidazole-1-sulfonic acid dimethylamide (48bi). Purification: Precipitation of the salt with 4 N HCl, washing the filtrate with dry diethyl ether. Yield: quantitative, based on (48ai). Yellow solid, m.p. 243° C.; ¹H NMR (400 MHz, DMSO-d₆) δ 2.81-2.84 (m, 2H, H-2); 2.98-3.00 (m, 2H, H-3); 6.76 (t, ⁴J=2.5 Hz, 1H, H-8); 7.22 (dd, ³J=8.5 Hz, ⁴J=1.9 Hz, 1H, H-6); 7.34 (d, ⁴J=1.6 Hz, 1H, H-4); 7.53 (d, ³J=8.5 Hz, 1H, H-7); 7.60 (s, 1H, H-13); 8.96 (s, 1H, H-11). IR (KBr) cm⁻¹: νₘₐₓ 3086, 2998, 2931, 2771, 2614, 1601, 1467, 1068, 830, 816. Anal. (C₁₃H₁₁ClN₂.HCl) C, H, N.

D) Purification Conditions, Yield and Characterization of the Title Compounds when Prepared According to the Comparative Synthesis A):

5-[(E)-2,3-Dihydro-1H-indane-1-ylidenemethyl]-1H-imidazole (42a). Purification: FCC (acetone); yield 28%, white solid, m.p. 148-151° C. ¹H NMR (400 MHz, DMSO-d₆, free base) δ 2.89-2.96 (m, 2H, H-2); 3.00-3.08 (m, 2H, H-3); 6.92 (t, ⁴J=2.5 Hz, 1H, H-8); 7.13-7.25 (m, 3H, H-5, H-6, H-13); 7.30 (d, ³J=6.5 Hz, 1H, H-4); 7.57 (d, ³J=6.8 Hz, 1H, H-7); 7.69 (s, 1H, H-11). IR (KBr, free base) cm⁻¹: νₘₐₓ 3055, 3020, 2960, 2920, 2840, 1643, 1601, 1460, 985, 757. Anal. (C₁₃H₁₂N₂.C₂H₂O₄) C, H, N.

5-[(Z)-2,3-Dihydro-1H-ind-1-ylidenemethyl]-1H-imidazolium oxalate (42b). Purification: FCC (CHCl₃:DMF, 9:2). Yield 100%, white solid, m.p. 196° C. ¹H NMR (400 MHz, DMSO-d₆) δ 2.85-2.94 (m, 4H, H-2, H-3); 6.36 (s, 1H, H-8); 7.14 (t, ³J=7.4 Hz, 1H, H-5); 7.24 (t, ³J=7.4 Hz, 1H, H-6); 7.31 (d, ³J=7.4 Hz, 1H, H-4); 7.37 (s, 1H, H-13); 8.13 (d, ³J=7.4 Hz, 1H, H-7); 8.30 (s, 1H, imidazole-H-11). IR (KBr) cm⁻¹: νₘₐₓ 1610, 1450, 750, 720. Anal. (C₁₃H₁₂N₂.0.75C₂H₂O₄) C, H, N.

5-[(E)-(5-Chloro-2,3-dihydro-1H-indene-1-ylidene)methyl]-1H-imidazolium chloride (48a). Purification: FCC (CHCl₃:DMF, 9:2). Yield 37%, white solid, m.p. 238° C. ¹H NMR (400 MHz, DMSO-d₆) δ 2.95-2.97 (m, 2H, H-2); 3.10-3.13 (m, 2H, H-3); 6.94 (s, 1H, H-8); 7.34 (d, ³J=8.3 Hz, 1H, H-6); 7.46 (s, 1H, H-4); 7.64 (d, ³J=8.3 Hz, 1H, H-7); 7.72 (s, 1H, H-13); 9.11 (s, 1H, H-11). IR (KBr) cm⁻¹: νₘₐₓ 3080, 1600, 1470, 1290, 1200, 1110, 1070, 830, 610. Anal. (C₁₃H₁₁ClN₂.HCl) C, H, N.

5-[(Z)-(5-Chloro-2,3-dihydro-1H-indene-1-ylidene)methyl]-1H-imidazolium oxalate (48b). Purification: FCC (CHCl₃:DMF, 9:2). Yield 17%, white solid, m.p. 218° C. ¹H NMR (400 MHz, DMSO-d₆) δ 2.87-2.96 (m, 4H, H-2, H-3); 6.39 (s, 1H, H-8); 7.20 (dd, ³J=8.4 Hz, ⁴J=2.0 Hz, 1H, H-6); 7.37-7.38 (m, 2H, H-4, H-13); 8.24 (s, 1H, H-11); 8.50 (d, ³J=8.4 Hz, 1H, H-7). IR (KBr) cm⁻¹: νₘₐₓ 1600, 1470, 1210, 880, 860, 830, 710. Anal. (C₁₃H₁₁ClN₂.C₂H₂O₄) C, H, N.

### Example 4

### General Working Protocol for the Synthesis of Compound 50

A solution of 20 9 of 1,2-dihydroacenaphthylene (130 mmol) in 250 ml of acetanhydride was stirred at 10° C. (cryostate). After slowly adding 10.4 ml (150 mmol) of concentrated nitric acid dropwise, the reaction mixture was stirred at 10° C. for another 20 h. After completion of the reaction and filtration of the yellow precipitate, recrystallization was effected in ethanol/ethyl acetate. The nitration product containing 3-nitro-1,2-dihydro-acenaphthylene was purified from the educt by means of column chromatography (FCC: EtOAc/hexane, 1:1). Yield of the nitration product: 71%, yellow solid.

Subsequently to the nitration, the hydrogenation of the nitration product was performed immediately. Thus, 13.8 g of the nitration product (69.3 mmol) was suspended in 250 ml of THF together with 1.3 g (10% by weight) of platinum on active charcoal (5%), and H₂ gas was applied at room temperature until no consumption of hydrogen could be established anymore. After filtrating the solution, the solvent was distilled off. The reaction was monitored by preparative TLC (twice developed in a mixture of EtOAc/hexane 1:1), and the two strongly fluorescing spots could be analyzed by means of ¹H NMR. The remaining solution was subjected to flash column chromatography in EtOAc/hexane (1:1). Again, only a mixture containing 1,2-dihydro-acenaphthylene-3-amine could be obtained. Yield of the mixture: 88%, brown solid.

1 2-Dihydro-acenaphthylene-3-amine. Purification: FCC (EtOAc:hexane, 1:1). ¹H NMR (500 MHz, DMSO-d₆) δ 3.09-3.12 (m, 2H, H-1); 3.29-3.32 (m, 2H, H-2); 5.10 (s, 2H, NH₂); 6.95-6.97 (d, ³J=8.5 Hz, 1H, H-7); 7.06-7.11 (m, 2H, H-3, H-4); 7.39-7.41 (m, 2H, H-5, H-6).

In the subsequent Sandmeyer reaction, 2.5 g of the amine mixture (15.7 mmol) was dissolved in 8.3 ml of conc. HBr and cooled down to 0° C. With vigorous stirring, about 7 ml of a 2.5 M NaNO₂ solution (17.25 g in 100 ml of water) was slowly added dropwise without exceeding a temperature of 5° C., until the reaction of the solution with iodine-starch paper was positive. Excess nitrous acid had to be destroyed with some spatula tip-fuls of urea. In a second flask, 3.0 g of copper bromide (21 mmol) was dissolved in 15 ml of conc. HBr at 0° C., and a layer of 30 ml of toluene was placed on top. It was required that the previously prepared solution of the diazonium salt was added quickly to the copper bromide solution, and that the mixture was allowed to stand at 0° C. with vigorous stirring for 10 min. After heating the reaction solution at 100° C. and reaction with reflux for 2 hours, the mixture was diluted with toluene and water and repeatedly extracted. The organic phase was dried over MgSO₄, and the solvent was distilled off. The obtained mixture of products which contained 3-bromo-1,2-dihydro-acenaphthylene could be isolated as a brown oil.

Yield of the mixture of products: 53%, brown oil.

In the subsequent Suzuki coupling, 0.425 g of the mixture (4.3 mmol), 15 ml of a 2 M Na₂CO₃ solution, 0.268 g of 3-pyridineboronic acid (5.148 mmol) and 0.106 g of tetrakis(triphenylphosphine)palladium (0.215 mmol) were suspended in methanol and boiled under reflux for 12 h under a nitrogen atmosphere. After extracting the mixture with dichloromethane and water and drying the organic phase over MgSO₄, the solvent was distilled off. From the thus obtained raw product, 3-(1,2-dihydroacenaphthylene-3-yl)pyridine (50) was isolated by FCC (EtOAc/hexane 1:1 as the mobile solvent). The light brown oil obtained was dissolved in anhydrous ether and admixed with HCl in ether. The hydrochloride precipitated quantitatively and could be filtered off.

3-(1,2-Dihydroacenaphthylene-3-yl)pyridine (50). Purification: FCC (EtOAc:hexane, 1:1). Yield: 360%, beige solid, m.p. 208° C. ¹H NMR (500 MHz, DMSO-d₆): δ=3.38-3.41 (m, 2H, H-2); 3.51-3.53 (m, 2H, H-3); 7.37-7.79 (m, 6H, H-3, H-4, H-5, H-6, H-7, H-13); 8.07 (dt, ³J=7.9 Hz, ⁴J=1.5 Hz, 1H, H-14); 8.58 (dd, ³J=4.8 Hz, ³J=1.5 Hz, 1H, H-12); 8.88 (d, ³J=2.4 Hz, 1H, H-10). IR cm⁻¹: νₘₐₓ 2971, 2901, 1612, 1578, 1550, 1449, 1329, 1261, 1065, 1048, 805. Anal. (C₂₁H₁₇N.HCl): 232.16 [M+H]⁺.

### Example 5

### Enzyme Test Systems for Testing Compounds for Inhibition of CYP Enzymes In Vitro

The following CYP enzymes were prepared and tested by the methods described: human CYP17 (recombinantly expressed in E. coli) (Hutschenreuter, T. U. et al., J. Enzyme Inhib. Med. Chem. 19: 17-32 (2004)), human placental CYP19 (Hartmann, R. W. & Batzl, C., J. Med. Chem. 29: 1362-1369 (1986)) and bovine adrenal CYP11B (Hartmann, R. W. et al., J. Med. Chem. 38: 2103-2111 (1995)).

A) Isolation of the CYP17-Containing Membrane Fraction from E. coli pJL17/OR

The recombinantly altered E. coli strain pJL17/OR, in which human CYP17 and rat NADPH-P450-reductase were coexpressed, was grown and stored according to the method of Ehmer et al. (Ehmer, P. B. et al., J. Steroid Biochem. Mol. Biol. 75: 57-63 (2000)). For isolating the membrane fraction, 5 ml of the bacterial cell suspension with an OD₅₇₈ of 50 was washed with phosphate buffer (0.05 M; pH 7.4; 1 mM MgCl₂; 0.1 mM EDTA and 0.1 mM DTT). The bacteria were removed by centrifugation and resuspended in 10 ml of ice cold TES buffer (0.1 M tris-acetate; pH 7.8; 0.5 mM EDTA; 0.5 M sucrose). Four milligrams of lysozyme in 10 ml of ice cold water was added to obtain a final concentration of 0.2 mg/ml. This was followed by incubation for 30 minutes under continued shaking on ice. The spheroblasts were obtained by a renewed centrifugation step at 12,000 g for 10 min and again resuspended in 3 ml of ice cold phosphate buffer (composition see above, additionally 0.5 mM PMSF).

After freezing and thawing, the cells were lysed on ice with an ultrasonic disintegrator. The whole cells and the cell debris were centrifuged off at 3000 g for 7 min. The supernatant was again centrifuged at 50,000 g for 20 min at 4° C. A membrane pellet sedimented and was resuspended in 2 ml of phosphate buffer (composition see above) with 200% glycerol by means of an Ultra-Turrax mixer. The protein concentration was determined by the method of Lowry et al. (Lowry, O. H. et al., J Biol Chem 193: 265-275 (1951)). Aliquots with an approximate protein concentration of 5 mg/ml were stored at −70° C. until use.

### B) Isolation of CYP19 (Aromatase)

The enzyme was obtained from the microsome fraction of fresh human placenta (St. Josephs Krankenhaus, Saarbrücken-Dudweiler, Germany) according to the method of Thompson and Siiteri (Thompson, E. A. & Siiteri, P. K., J. Biol. Chem. 249: 5364-5372 (1974)). The isolated microsomes were suspended in a minimum volume of phosphate buffer (0.05 M; pH 7.4; 200% glycerol). In addition, DTT (10 mM) and EDTA (1 mM) were added to protect the enzyme from degradation reactions. The protein concentration was determined according to Lowry et al. (Lowry, O. H. et al., J. Biol. Chem. 193: 265-275 (1951)) and should be about 35 mg/ml after the processing.

C) Processing of bovine CYP11B

Bovine adrenal glands obtained freshly after slaughtering were stored in ice cold Tris-sucrose buffer (0.25 M sucrose, 0.05 M Tris; pH 7.4) until processed. After removing the appending adipose tissue, the adrenal medulla was carefully separated from the adrenal cortex using scissors. The pieces of adrenal cortex were coarsely comminuted with scissors, washed with Tris-sucrose buffer, weighed and finely comminuted in the above buffer (2 ml per g of tissue) with a hand mixer. Thereafter, the tissue was homogenized with an Ultra-Turrax mixer. For separating coarse cell debris and the nuclei, the homogenizate was centrifuged twice for 15 min at 900 g and 4° C. The supernatant was subsequently centrifuged for 35 min at 11,000 g for recovering the mitochondrial fraction. For purifying the mitochondrial fraction, the precipitate was resuspended in Tri-sucrose buffer and again centrifuged at 11,000 g for 35 min. This washing step was performed two times in total. After the last centrifugation, the pellet was resuspended in Tris-sucrose buffer containing 0.001 M EDTA and frozen at −70° C. Before the enzyme was employed in a CYP11B inhibition assay, the mitochondrial suspension was diluted with 18-hydroxylase buffer (0.05 M Tris, 1.2 mM MgCl₂, 6.0 nM KCl, 140 mM NaCl, 2.5 mM CaCl₂) to a protein concentration of 5 mg/ml (Ayub, M. & Levell, M. J., 3. Steroid Biochem. 32: 515-524 (1989)). The protein determination was performed according to Lowry (Lowry, O. H. et al., J. Biol. Chem. 193: 265-275 (1951)).

### D) Determination of Percent Inhibition of CYP17

A solution of 6.25 nmol of progesterone (in 5 μl of MeOH) was dissolved in 140 μl of phosphate buffer (0.05 M; pH 7.4; 1 mM MgCl₂; 0.1 mM EDTA and 0.1 mM DTT) and preincubated for 5 min at 37° C. together with 50 μl of NADPH-regenerating system (phosphate buffer with 10 mM NADP^{⊕}, 100 mM glucose-6-phosphate and 2.5 units of glucose-6-phosphate dehydrogenase) and inhibitor (in 5 μl of DMSO). Control incubations were performed in parallel with 5 μl DMSO without urea. The reaction was started by adding 50 μl of a membrane suspension diluted 1 to 5 in phosphate buffer (0.8 to 1 mg of protein per ml). After thoroughly mixing the components, the mixture was incubated at 37° C. for 30 min. The reaction was quenched by adding 50 μl of 1 N HCl.

The steroids were extracted with 1 ml of EtOAc. After a centrifugation step (5 min at 2,500 g), 900 μl of the organic phase was transferred into an Eppendorf vessel with 250 μl of the incubation buffer and 50 μl of 1 N HCl and again shaken. After the centrifugation, 800 μl of the organic phase was removed, placed into a new vessel and evaporated to dryness. The samples were dissolved in 50 μl of a water-methanol mixture (1:1) and analyzed by HPLC. The substrate conversion was calculated from the ratio of the areas of the product peaks (17α-hydroxyprogesterone and 16α-hydroxyprogesterone) to that of the substrate peak. The activity of the inhibitors was calculated from the reduced substrate conversion after the addition of inhibitors in accordance with the following formula:

$$\% inhibition = [ [ \frac{\sum peak areas ( inhibitor incubation )}{\sum peak areas ( control incubation )} ] - 1 ] \cdot ( - 100 )$$

### E) Determination of the IC₅₀ of CYP19

The assay was performed by approximate analogy with the test methods described by Foster et al. and Graves and Salahanick; a detailed description can be found in Hartmann and Batzl 1986 (Foster, A. B. et al., J Med Chem 26: 50-54 (1983); Graves, P. E. & Salhanick, H. A., Endocrinology 105: 52-57 (1979); Hartmann, R. W. & Batzl, C., J. Med. Chem. 29: 1362-1369 (1986)). The enzyme activity was monitored by measuring the ³H₂O formed from [1β-³H]androstenedione during the aromatization. Each reaction vessel contained 15 nM of radioactively labeled [1β-³H]androstenedione (corresponding to 0.08 μCi) and 485 nM of unlabeled androstenedione, 2 mM NADP^{⊕}, 20 mM glucose-6-phosphate, 0.4 units of glucose-6-phosphate dehydrogenase and inhibitor (0-100 μM) in phosphate buffer (0.05 M; pH 7.4). The compounds to be tested were dissolved in DMSO and diluted with buffer to the desired concentration. The final DMSO concentration of the control and inhibitor incubations was about 20%. Each vessel was preincubated in a water bath at 30° C. for 5 min. The reaction was started by adding the microsomal protein (0.1 mg). The total volume of each mixture was 200 μl. After 14 min, the reaction was quenched by adding 200 μl of ice cold 1 mM HgCl₂ solution. Two hundred microliters of a 2% aqueous suspension of dextran-coated charcoal, DCC) was added for absorbing the steroids, and the vessels were shaken for 20 min. Thereafter, the charcoal was centrifuged off at 1500 g for 5 min. The radioactive water present in the supernatant (³H₂O) was assayed by scintillation measurement using an LKB-Wallac beta counter. The calculation of the IC₅₀ values was effected by a semilogarithmic plot of the percent inhibition against the inhibitor concentration. From this plot, the molar concentration at which 50% inhibition occurred was read.

### F) Determination of the Percent Inhibition of Bovine CYP11B

The substrate corticosterone was dissolved in methanol and diluted with Tris buffer to a final concentration of 200 μM. The inhibitors were also dissolved in methanol and diluted with buffer to a final concentration of 1 μM. The concentration of methanol in the incubation was 2.4% in an incubation volume of 0.5 ml. Corticosterone (200 μM) was incubated with inhibitor (1 μM) and mitochondrial enzyme (0.5 mg/0.5 ml) with the addition of a regenerating system consisting of NADP⁺ (1 mM), glucose-6-phosphate (7 mM) and glucose-6-Phosphate dehydrogenase (1 IU/0.5 ml). Up to 5 inhibitors per test could be incubated in duplicate. Four control and 2 blind reactions contained a corresponding volume of buffer and methanol (2%) instead of the inhibitor solution. After preincubation for 5 minutes (30° C.; water bath), the reaction was started by adding mitochondrial suspension. The enzymatic reaction was quenched after an incubation time of 10 min by adding 250 μl of 1 N HCl. The blind values were admixed with 250 μl of HCl before the enzyme was pipetted in. The steroids were separated off by shaking with 1 ml of ethyl acetate (10 min). After centrifugation (15,000 g, 10 min), 900 μl of the organic phase was shaken with 250 μl 1 N NaOH (10 min), and 800 μl of supernatant was again washed with 250 μl of buffer. After evaporating 700 μl of the organic phase, the steroids were taken up in 20 μl of distilled methanol, and 10 μl each of the methanolic solution was separated by means of HPLC (stationary phase: Nucleosil 120-5 C18 column with a 7 μm precolumn of 1 cm length; mobile solvent: 50% methanol in water; flow rate: 1.1 ml/min; detection: UV detector). 18-OH-corticosterone (retention time: 10 min) and corticosterone (retention time: 21 min) were separated. The height of the 18-OH-corticosterone peak was used for evaluation. The percent inhibition of the 18-hydroxylation of corticosterone by the inhibitors was based on the mediums of the control incubation, taking the blind values into account. Each inhibitor was tested at least two times for its 18-hydroxylase inhibitory activity at a concentration of 1 μM. The determination of the amounts produced of 18-OH-corticosterone for testing the incubation time and the substrate saturation was effected by means of a straight calibration line.

### Example 6

### Biological Test Systems for the Testing of Compounds for Selective Inhibition of Human CYP11B1and CYP11B2 In Vitro

A) Screening test in transgenic fission yeast: A suspension of fission yeast (S. pombe PE1) with a cell density of 3·10⁷ cells/ml was prepared on a freshly grown culture using fresh EMMG (pH 7.4) as modified according to Ehmer et al. (Ehmer, P. B. et al., J. Steroid. Biochem. Mol. Biol. 81, 173-179 (2002)). 492.5 μl of this cell suspension was admixed with 5 μl of inhibitor solution (50 μM of the compound to be tested in ethanol or DMSO) and incubated at 32° C. for 15 min. Controls were admixed with 5 μl of ethanol. The enzyme reaction was started by adding 2.5 μl of 11-deoxycorticosterone (20 μM, containing 1.25 nCi of [4-¹⁴C]11-deoxycorticosterone in Ethanol), followed by horizontal shaking at 32° C. for 6 h. The test was quenched by extracting the sample with 500 μl of EtOAc. After centrifugation (10,000 g, 2 min), the EtOAc phase was removed and evaporated to dryness. The residue was taken up in 10 μl of chloroform. The reaction of the substrate to form corticosterone was analyzed by HPTLC (see below).

The quantification of the spots for the substrate deoxycorticosterone and the products corticosterone (and, if detectable, 18-hydroxycorticosterone and aldosterone) was effected with the related evaluation program AIDA. For the human aldosterone synthase expressed in S. pombe, only corticosterone as a product and the substrate deoxycorticosterone were detected. At an incubation time of 6 hours, 18-hydroxycorticosterone and aldosterone were not formed at any detectable concentrations and therefore were not included in the evaluation. The calculation of the conversion rate was effected according to equation 1.

$$\% P = \frac{[ P S {L}_{B} ] - P S {L}_{HG}}{[ P S {L}_{DOC} + P S {L}_{B} ] - 2 \times P S {L}_{HG}} \times 100 \qquad Equation 1$$

% P conversion rate (percent proportion of the product to the total steroid)
PSL phospho-stimulated luminescence (luminescence value)
PSL_{B} PSL for corticosterone (B)
PSL_{DOC} PSL for deoxycorticosterone (DOC)
PSL_{HG} PSL of the background

The percent inhibition caused by an inhibitor in the respectively employed concentration was calculated according to equation 2.

$$\% H = [ 1 - \frac{\% {P}_{H}}{\% {P}_{K}} ] \times 100 \qquad Equation 2$$

% H percent inhibition
% P percentage of conversion of the substrate to products
% P_{H} percent conversion in the absence of an inhibitor
P_{K} percent conversion of the control

### B) Test for Selective CYP11B1 and CYP11B2 Inhibitors:

Maintenance of cells: V79 MZh11B1 and V79 MZh11B2, which recombinantly express human aldosterone synthase and steroid-11-β hydroxylase, respectively, and were prepared according to Denner et al. (Denner, K. et al., Pharmacogenetics 5: 89-96 (1995)) were cultured in a CO2 incubator at 37° C. and in a water vapor-saturated atmosphere with 5% CO2 in cell culture dishes of 60 or 90 mm diameter. Both cell lines were cultured in DMEM⁺ containing 100% FCS and the antibiotics penicillin and streptomycin (10%) for protection from bacterial contamination. The cells were passaged every 2-3 days after treatment with trypsin/EDTA because the doubling rate was 1-2 days depending on the number of cells. The cells were passaged for a maximum of 12-15 times in order to exclude any cell alterations. When there was further need, freshly thawed cells were employed.

DMEM⁺ - medium

| | | |
|---|---|---|
| DMEM powder medium | 13.4 | g |
| NaHCO₃ | 3.7 | g |
| L-Glutamine (200 mM) | 20.0 | ml |
| Penicillin (100 units/ml)/streptomycin (0.1 mg/ml) | 10.0 | ml |
| Sodium pyruvate (100 mM) | 10.0 | ml |
| Fetal calf serum (FCS) | 100 | ml |
| H₂O bidist. | ad 1 | l |

The pH of the mediums was adjusted to 7.2-7.3. FCS was added only after sterile filtration.

Inhibition test: V79 MZh 11B1 and V79 MZh 11B2 cells (8·10⁵ cells per well) were grown to confluency on 24-well cell culture plates with 1.9 cm² culture area per well (Nunc, Roskilde, Denmark). Before the test, the DMEM culture medium present was removed, and 450 μl of fresh DMEM with inhibitor was added for at least three concentrations to each well to determine the IC₅₀. After preincubation (60 min, 37° C.), the reaction was started by adding 50 μl of DMEM with 2.5 μl of solution of the substrate 11-deoxycorticosterone (20 μM, containing 1.25 nCi of [4-¹⁴C]11-deoxycorticosterone in ethanol). Thereafter, the plate was stored at 37° C. and 50% CO₂ in a CO₂ incubator. The V79 MZh 11B1 cells were incubated for 120 min, and the V79 MZh 11B2 cells were incubated for 40 min. Controls without inhibitor were treated in the same way. The enzyme reactions were quenched by extracting the supernatant with 500 μl of EtOAc. The samples were centrifuged (10,000 g, 2 min), the solvent was removed and evaporated. The residue was taken up in 10 μl of chloroform and analyzed by HPTLC (see below).

The conversion rate for V79 MZh 11B1 was calculated by analogy with equation 1 (Ex. 5A), where:

PSL_{B} PSL for cortisol or corticosterone
PSL_{DOC} PSL for deoxycortisol (RSS) or deoxycorticosterone

For V79 MZh11B2, the conversion rate was obtained in accordance with equation 3:

$$\% P = \frac{[ P S {L}_{B} + P S {L}_{18 OHB} + P S {L}_{Aldo} ] - 3 \times P S {L}_{HG}}{[ \begin{matrix}P S {L}_{DOC} + P S {L}_{B} + \\ P S {L}_{18 OHB} + P S {L}_{Aldo}\end{matrix} ] - 4 \times P S {L}_{HG}} \times 100 \qquad Equation 3$$

% P conversion rate (proportion of product to the total steroid)
PSL phospho-stimulated luminescence (luminescence value)
PSL_{B} PSL for corticosterone (B)
PSL_{18OHB} PSL for 18-hydroxycorticosterone (18OHB)
PSL_{Aldo} PSL for aldosterone
PSL_{DOC} PSL for 11-deoxycorticosterone (DOC)
PSL_{H G} PSL of the background

The percent inhibition caused by an inhibitor in the respectively employed concentration was calculated according to equation 2 (Ex. 5A).

Determination of the IC₅₀: The IC₅₀ is defined as that concentration of the inhibitor at which the enzyme is inhibited by 50%. It was calculated by determining the percent inhibition for at least 3 different inhibitor concentrations, which must all be in the linear range of the sigmoidal IC₅₀ curve (log C/% inhibition).

The calculation was effected by linear regression. The values determined were used only if they formed a straight line with a reliability of r<0.95.

C) HPTLC analysis and phospho-imaging of the radioactively labeled steroids: The resuspended residue from Example 6A or 6B which contained the radioactively labeled steroids was applied to an HPTLC plate (20×10 cm, silica gel 60F₂₅₄) with a concentration zone (Merck, Darmstadt, Germany). The plate was developed twice with the mobile solvent chloroform:methanol:water (300:20:1). Unlabeled 11-deoxycorticosterone and corticosterone were applied as a reference for the CYP11B1 reaction. For the CYP11B2 reaction, 11-deoxycorticosterone, corticosterone, 18-hydroxycorticosterone and aldosterone were used as references. The detection of the unlabeled references was effected at 260 nm. Subsequently, imaging plates (BAS MS2340, for ¹⁴C samples, Raytest, Straubenhardt, Germany) were exposed to the HPTLC plates for 48 h. The imaging plates were scanned with the phosphoimager system Fuji FLA 3000 (Raytest, Straubenhardt, Germany), and the steroids were quantified.

### Example 7

### Inhibition of Adrenal CYP11B Enzymes In Vitro by [dihydronaphthalene- or dihydroindane-1(2H)-ylidenemethyl]-3-pyridines

[Dihydronaphthalene- or dihydroindane-1(2H)-ylidenemethyl]-3-pyridines were tested as inhibitors as described in Examples 5 and 6. The results of the tests are summarized in Table 1.

TABLE 1
[Dihydronaphthalene- or dihydroindane-1(2H)-ylidenemethyl]-3-pyri-
dines: inhibition of adrenal CYP11B enzymes, CYP17 and CYP19 in vitro

| 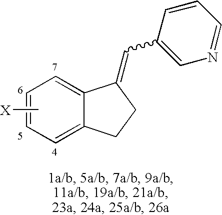 | | | | 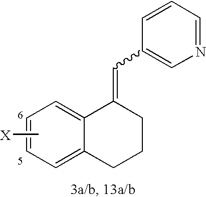 | | | |
|---|---|---|---|---|---|---|---|
| | | | % Inhibition^{a} | IC₅₀ (nM)^{c} | | % Inhibition^{a} | IC₅₀ (μM)^{g} |
| | | | human^{b} | V79 11B1^{d} | V79 11B2^{d} | human^{f} | human^{h} |
| Compound | X | Isomer | hCYP11B2 | hCYP11B1 | hCYP11B2 | CYP17 | CYP19 |
| 1a | H | E | 82 | 888.2 | 10.6 | 14 | 2.04 |
| 1b | H | Z | 83 | 86.6 | 92.0 | 33 | 3.55 |
| 3a | H | E | 70 | 715.4 | 21.6 | 9 | 6.58 |
| 3b | H | Z | 68 | 1424.1 | 141.4 | 24 | 3.79 |
| 5a | 5-F | E | 88 | 310.7 | 6.8 | 17 | 2.54 |
| 5b | 5-F | Z | 89 | 125.0 | 10.8 | 16 | 2.49 |
| 7a | 5-Cl | E | 75 | 1472.0 | 26.2 | 26 | 4.26 |
| 7b | 5-Cl | Z | 68 | 269.5 | 72.7 | 28 | 4.05 |
| 9a | 5-Br | E | 60 | 1936.9 | 37.4 | 16 | 7.83 |
| 9b | 5-Br | Z | 58 | 319.9 | 170.9 | 24 | 7.37 |
| 11a | 5-OMe | E | 79 | 1448.3 | 34.2 | 36 | 4.31 |
| 11b | 5-OMe | Z | 81 | 790.4 | 26.4 | 49 | 5.57 |
| 13a | 6-OMe | E | 52 | 903.1 | 56.6 | 20 | 1.13 |
| 13b | 6-OMe | Z | 53 | 206.0 | 877.6 | 17 | 4.00 |
| 19a | 5-OEt | E | 67 | 2368.3 | 79.4 | 13 | 7.08 |
| 19b | 5-OEt | Z | 52 | 2696.9 | 248.3 | 6 | 7.68 |
| 23a | 4-Me | E | 52 | 763.1 | 108.0 | 40 | 4.72 |
| 24a | 4-F | E | 84 | 773.5 | 20.9 | 42 | 1.81 |
| 25a | 4-Cl | E | 92 | 303.5 | 8.7 | 14 | 0.13 |
| 25b | 4-Cl | Z | 83 | 657.3 | 30.6 | 32 | 0.23 |
| 26a | 7-OMe | E | 76 | 954.8 | 26.7 | 5 | 9.74 |
| Ketoconazole | | | 36 | n.d. | 80.5 | 40 | n.d. |
| Fadrozole | | | 68 | 9.7 | 1.0 | 7 | 0.0295 |

^{a}Mean value of 4 determinations, standard error <10%.
^{b}S. pombe cells which express human CYP11B2; deoxycorticosterone substrate, 100 nM; inhibitor, 500 nM.
^{c}Mean value of 4 determinations, standard error <20%.
^{d}hamster fibro-blasts which express human CYP11B1; deoxycorticosterone substrate, 100 nM.
^{e}hamster fibroblasts which express human CYP11B2; deoxycorticosterone substrate, 100 nM.
^{f}E. coli which expresses human CYP17; 5 mg/ml protein; progesterone substrate, 2.5 μM; inhibitor, 2.5 μM.
^{g}Mean value of 4 determinations, standard error <5%;
^{h}human placental CYP19, 1 mg/ml protein; testosterone substrate, 2.5 μM; (n.d. = not determined)

### Example 8

### Inhibition of Adrenal CYP11B Enzymes In Vitro by [Dihydronaphthalene- or dihydroindane-1(2H)-ylidenemethyl]-4-pyridines

[Dihydronaphthalene- or dihydroindane-1(2H)-ylidenemethyl]-4-pyridines were tested as inhibitors as described in Examples 5 and 6. The results of the tests are summarized in Table 2.

TABLE 2
[Dihydroindane-1(2H)-ylidenemethyl]-4-pyridines.: Inhibition of adrenal
CYP11B enzymes, CYP17 and CYP19 in vitro

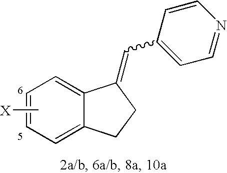

| | | | % Inhibition^{a} | IC₅₀ (nM)^{c} | | % Inhibition^{a} | IC₅₀ (μM)^{g} |
|---|---|---|---|---|---|---|---|
| | | | human^{b} | V79 11B1^{d} | V79 11B2^{d} | human^{f} | human^{h} |
| Compound | X | Isomer | hCYP11B2 | hCYP11B1 | hCYP11B2 | CYP17 | CYP19 |
| 2a | H | E | 55 | n.d. | n.d. | 8 | 6.70 |
| 2b | H | Z | n.d. | 1315.4 | 931.2 | 18 | 12.73 |
| 6a | 5-F | E | 37 | 379.7 | 1098.1 | 15 | 1.55 |
| 6b | 5-F | Z | 77 | 257.4 | 34.0 | 29 | 0.80 |
| 8a | 5-Cl | E | 38 | 243.1 | 1515.3 | 18 | 5.43 |
| 10a | 5-Br | E | 17 | 947.5 | 2640.1 | 24 | >36 |
| Ketoconazole | | | 36 | n.d. | 80.5 | 40 | n.d. |
| Fadrozole | | | 68 | 9.7 | 1.0 | 7 | 0.0295 |

^{a}Mean value of 4 determinations, standard error <10%.
^{b}S. pombe cells which express human CYP11B2; deoxycorticosterone substrate, 100 nM; inhibitor, 500 nM.
^{c}Mean value of 4 determinations, standard error <20%.
^{d}Hamster fibroblasts which express human CYP11B1; deoxycorticosterone substrate, 100 nM.
^{e}Hamster fibroblasts which express human CYP11B2; deoxycorticosterone substrate, 100 nM.
^{f}E. coli which expresses human CYP17; 5 mg/ml protein; progesterone substrate, 2.5 μM; inhibitor, 2.5 μM.
^{g}Mean value of 4 determinations, standard error <5%;
^{h}human placental CYP19, 1 mg/ml protein; testosterone substrate, 2.5 μM; (n.d. = not determined)

### Example 9

### Inhibition of Adrenal CYP11B Enzymes, CYP17 and CYP19 In Vitro by Other [Dihydroindane-1(2H)-ylidenemethyl] Heterocycles

Other [dihydroindane-1(2H)-ylidenemethyl] heterocycles were tested as inhibitors as described in Examples 5 and 6. The results of the tests are summarized in Table 3.

TABLE 3
Other [dihydroindane-1(2H)-ylidenemethyl]heterocycles: Inhibition of
adrenal CYP11B enzymes, CYP17 and CYP19 in vitro

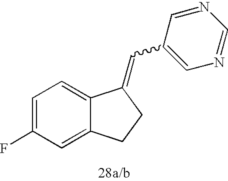

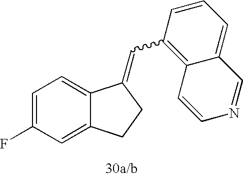

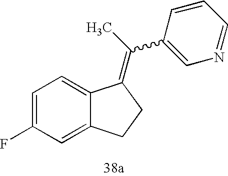

| | % | | | % | IC₅₀ |
|---|---|---|---|---|---|
| | Inhibition^{a} | IC₅₀ (nM)^{c} | | Inhibition^{a} | (μM)^{g} |
| | | V79 | V79 | | |
| Com- | human^{b} | 11B1^{d} | 11B2^{d} | human^{f} | human^{h} |
| pound | hCYP11B2 | hCYP11B1 | hCYP11B2 | CYP17 | CYP19 |
| 28a | 72 | 3178.5 | 27.4 | 6 | 7.45 |
| 30a | 60 | 1129.0 | 58.1 | 57 | 0.72 |
| 30b | 91 | 374.1 | 26.1 | 65 | 1.94 |
| 38a | 67 | 159.3 | 96.1 | n.d. | n.d. |
| Keto- | 36 | n.d. | 80.5 | 40 | n.d. |
| cona- | | | | | |
| zole | | | | | |
| Fadro- | 68 | 9.7 | 1.0 | 7 | 0.0295 |
| zole | | | | | |

^{a}Mean value of 4 determinations, standard error <10%.
^{b}S. pombe cells which express human CYP11B2; deoxycorticosterone substrate, 100 nM; inhibitor, 500 nM.
^{c}Mean value of 4 determinations, standard error <20%.
^{d}Hamster fibro-blasts which express human CYP11B1; deoxycorticosterone substrate, 100 nM.
^{e}Hamster fibroblasts which express human CYP11B2; deoxycorticosterone substrate, 100 nM.
^{f}E. coli which express human CYP17; 5 mg/ml protein; progesterone substrate, 2.5 μM; inhibitor, 2.5 μM.
^{g}Mean value of 4 determinations, standard error <5%;
^{h}human placental CYP19, 1 mg/ml protein; testosterone substrate, 2.5 μM; (n.d. = not determined)

### Example 10

### Inhibition of Adrenal CYP11B Enzymes and CYP17 and CYP19 In Vitro by [Dihydronaphthalene- or dihydroindane-1(2H)-ylidenemethyl]-4-imidazoles

[Dihydronaphthalene- or dihydroindane-1(2H)-ylidenemethyl]-4-imidazoles were tested as inhibitors as described in Examples 5 and 6. The results of the tests are summarized in Tables 4 and 5.

TABLE 4
Inhibition of adrenal CYP11B enzymes in vitro by [dihydronaphthalene-
or dihydroindane-1(2H)-ylidenemethyl]-4-imidazoles

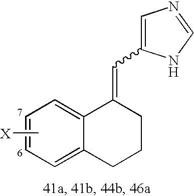

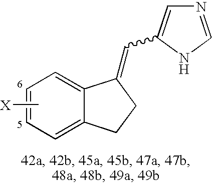

| | | | % Inhibition^{a} | | IC₅₀ (nM)^{d} | |
|---|---|---|---|---|---|---|
| | | | bovine^{b} | human^{c} | V79 11B1^{e} | V79 11B2^{f} |
| Compound | X | Isomer | bCYP11B | hCYP11B2 | hCYP11B1 | hCYP11B2 |
| 41a | H | E | 40 | 80 | 31.4 | 24.8 |
| 41b | H | Z | 95 | 82 | 3.3 | 9.6 |
| 42a | H | E | 62 | 77 | 25.9 | 41.0 |
| 42b | H | Z | 94 | 81 | 6.1 | 11.0 |
| 44b | 6-CN | Z | 97 | 54 | 6.90 | 22.7 |
| 45a | 5-CN | E | 81 | 49 | 15.0 | 35.9 |
| 45b | 5-CN | Z | 100 | 65 | 12.3 | 35.7 |
| 46a | 7-Cl | E | 66 | 48 | 18.7 | 47.3 |
| 47a | 5-F | E | 67 | 59 | 20.6 | 16.7 |
| 47b | 5-F | Z | 64 | 58 | 11.17 | 13.9 |
| 48a | 5-Cl | E | 45 | 79 | 28.7 | 88.8 |
| 48b | 5-Cl | Z | 87 | 80 | 19.5 | 3.7 |
| 49a | 5-Br | E | 63 | 68 | 26.2 | 92.8 |
| 49b | 5-Br | Z | 88 | 76 | 23.5 | 10.3 |
| Fadrozole | | | n.d. | 68 | 9.7 | 1.0 |
| Ketoconazole | | | 78 | 36 | n.d. | 80.5 |

^{a}Mean value of 4 determinations, standard error <10%.
^{b}bovine adrenal mitochondria, 1 mg/ml protein; corticosterone substrate, 200 μM; inhibitor, 1 μM (R. Hartmann et al., J. Med. Chem. 38, 2103-2111 (1995)).
^{c}S. pombe cells which express human CYP11B2; deoxycorticosterone substrate, 100 nM; inhibitor, 500 nM.
^{d}Mean value of 4 determinations, standard error <20%.
^{e}Hamster fibroblasts which express human CYP11B1; deoxycorticosterone substrate, 100 nM.
^{f}Hamster fibroblasts which express human CYP11B2; deoxycorticosterone substrate, 100 nM. (n.d. =not determined)

TABLE 5
Inhibition of CYP17 and CYP19 in vitro by [dihydronaphthalene- or
dihydroindane-1(2H)-ylidenemethyl]-4-imidazoles

| | | | % Inhibition | IC₅₀ (μM) |
|---|---|---|---|---|
| Compound | X | Isomer | CYP17^{a} | CYP19^{b} |
| 41a | H | E | 13 | 0.226 |
| 41b | H | Z | 13 | 0.190 |
| 42a | H | E | 11 | 0.955 |
| 42b | H | Z | 1 | 0.130 |
| 44b | 6-CN | Z | 4 | 0.125 |
| 45a | 5-CN | E | 21 | 0.119 |
| 45b | 5-CN | Z | 4 | 0.015 |
| 46a | 7-Cl | E | 24 | 0.120 |
| 47a | 5-F | E | 16 | 0.218 |
| 47b | 5-F | Z | 11 | 0.020 |
| 48a | 5-Cl | E | 37 | 0.330 |
| 48b | 5-Cl | Z | 26 | 0.039 |
| 49a | 5-Br | E | 13 | 0.027 |
| 49b | 5-Br | Z | 15 | 0.100 |
| Fadrozole | | | 7 | 0.005^{c} |
| Ketoconazole | | | 40 | n.d. |

^{a}Mean value of 4 determinations, standard error <10%; E. coli which express human CYP17; 5 mg/ml protein; progesterone substrate, 2.5 μM; inhibitor, 2.5 μM.
^{b}Mean value of 4 determinations, standard error <5%; human placental CYP19, 1 mg/ml protein; testosterone substrate, 2.5 μM;
^{c}according to Bhatnagar, A. S. et al., J. Steroid Biochem. Mol. Biol. 37: 363-367 (1990);
(n.d. = not determined)

### Example 11

### Inhibition of CYP Enzymes In Vitro by the Reference Compounds Ketoconazole and Fadrozole

Ketocoanzole or fadrozole were tested as inhibitors as described in Examples 5 and 6. The results of the tests are summarized in Table 6.

TABLE 6
Ketoconazole and fadrozole: Inhibition of adrenal CYP11B enzymes,
CYP17 and CYP19 in vitro

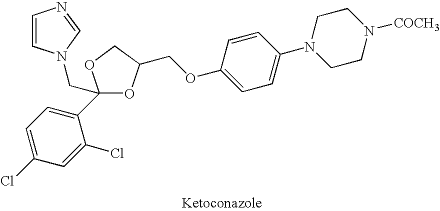

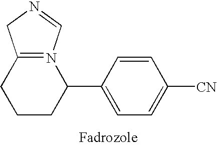

| | | | | % Inhi- | |
|---|---|---|---|---|---|
| | % Inhibition^{a} | IC₅₀ (nM)^{c} | | bition | IC₅₀ (nM) |
| Com- | human^{b} | V79 11B1^{d} | V79 11B2^{e} | human^{f} | human^{g} |
| pound | hCYP11B2 | hCYP11B1 | hCYP11B2 | CYP17 | CYP19 |
| Keto- | 36 | n.d. | 80.5 | 40 | n.d. |
| cona- | | | | | |
| zole | | | | | |
| Fadro- | 68 | 9.7 | 1.0 | 7 | 29.5 |
| zole | | | | | |

^{a}Mean value of 4 determinations, standard error <10%;
^{b}S. pombe cells which express human CYP11B2; deoxycorticosterone substrate, 100 nM; inhibitor, 500 nM.
^{c}Mean value of 4 determinations, standard error <20%.
^{d}Hamster fibroblasts which express human CYP11B1; deoxycorticosterone substrate, 100 nM.
^{e}Hamster fibroblasts which express human CYP11B2; deoxycorticosterone substrate, 100 nM.
^{f}Mean value of 4 determinations, standard error <10%; E. coli which express human CYP17; 5 mg/ml protein; progesterone substrate, 2.5 μM; inhibitor, 2.5 μM.
^{g}Mean value of 4 determinations, standard error <5%; human placental CYP19, 1 mg/ml protein; testosterone substrate, 2.5 μM; (n.d. = not determined)

### Example 12

### Test of Selected Compounds with NCI-H295R Cells

Of the compounds presented under Examples 6 and 7, one was examined on the NCI-H295R system. For comparison, fadrozole was used as a reference. The exemplary results obtained are not directly comparable with the IC₅₀ values and percent inhibition values obtained in V79 cells since other test parameters and a different substrate, inter alia, were used for the inhibitor assays on NCI-H295R (explanation see Table 7).

In comparison with fadrozole, a coarse correlation between the two test systems could be established.

TABLE 7
Comparison of inhibition data from NCI-H295R, S. pombe PE1, V79 MZ

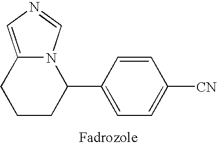

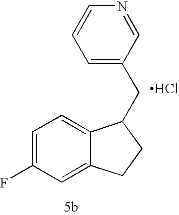

| | NCI- | NCI- | NCI- | | | |
|---|---|---|---|---|---|---|
| | H295R | H295R | H295R | S. pombe | | |
| | CYP11B1 | CYP11B2 | CYP11B2 | PE1 | | |
| | RSS | B | DOC | CYP11B2 | V79MZh11B1 | V79MZh11B2 |
| | % | % | % | DOC | CYP11B1 | CYP11B2 |
| Com- | inhibition | inhibition | inhibition | % | DOC | DOC |
| pound | [IC₅₀]^{a} | [IC₅₀]^{b} | [IC₅₀]^{c} | inhibition^{d} | [IC₅₀]^{e} | [IC₅₀]^{f} |
| Fadrozole | 92% | 35.4% | 89.6% | | [9 nM] | [1 nM] |
| | [23.8 nM] | [23.6 μM] | [18.7 nM] | | | |
| 5b | 73.1% | 6.9% | 89.2% | 88.6% | [124.9 nM] | [10.8 nM] |
| | | [17 μM] | [155.6 nM] | | | |

^{a}percent inhibition of CYP11B1 in NCI-H295R, inhibitor concentration 2.5 μM (for determination of IC₅₀: at least 3 different concentrations); preincubation 1 h, substrate: [³H]-deoxycortisol (RSS, 500 nM); incubation time: 48 h; extraction with dichloromethane; determination of cortisol after HPLC separation (methanol-water 50:50; RP18)
^{b}percent inhibition of CYP11B2 in NCI-H295R, inhibitor concentration 2.5 μM (for determination of IC₅₀: at least 3 different concentrations); stimulation with salt solution containing K⁺ ions [20 mM K⁺] preincubation 1 h, substrate: [³H]-corticosterone (B, 500 nM); incubation time: 24 h; extraction with dichloro-methane; determination of [³H]18-hydroxycorticosterone and [³H]aldosterone after HPTLC separation(chloroform-methanol-water 300:20:1; phosphoimager)
^{c}percent inhibition of CYP11B2 in NCI-H295R, inhibitor concentration 2.5 μM (for determination of IC₅₀: at least 3 different concentrations); preincubation 1 h, substrate: [¹⁴C]deoxycorticosterone (DOC, 500 nM); incubation time: 3 h; extraction with dichloromethane; determination of [¹⁴C]corticosterone, [¹⁴C]18-hydroxycorticosterone and [¹⁴C]aldosterone after HPTLC separation (chloroform-methanol-water 300:20:1; phosphoimager)
^{d}percent inhibition of CYP11B2 in S. pombe PE1, inhibitor concentration 500 nM; preincubation 1 h, substrate: [¹⁴C]deoxycorticosterone (DOC, 100 nM); in-cubation time: 6 h; extraction with ethyl acetate; determination of [¹⁴C]corticosterone after HPTLC separation (chloroform-methanol-water 300:20:1; phospho-imager)
^{e}determination of IC₅₀ for CYP11B1 in V79MZh11B1; determination of IC₅₀ in at least 3 different inhibitor concentrations; preincubation 1 h, substrate: [¹⁴C]deoxycorticosterone (DOC, 100 nM); incubation time: 140 min; extraction with ethyl acetate; determination of [¹⁴C]corticosterone after HPTLC separation (chloroform-methanol-water 300:20:1; phosphoimager)
^{f}determination of IC₅₀ for CYP11B2 in V79MZh11B2; determination of IC₅₀ in at least 3 different inhibitor concentrations; preincubation 1 h, substrate: [¹⁴C]deoxycorticosterone (DOC, 100 nM); incubation time: 40 min; extraction with ethyl acetate; determination of [¹⁴C]corticosterone, [¹⁴C]18-hydroxycorticosterone and [¹⁴C]aldosterone after HPTLC separation (chloroform-methanol-water 300:20:1; phosphoimager)

For examining the effect of different inhibitors on NCI-H295R, a test method in a 24-well format has been developed. For testing inhibitors, a preincubation for one hour was performed first, followed by starting the enzyme reactions by adding substrate (500 nM).

A) Seeding: The cell lines were grown and passaged until a confluent cell lawn had formed. By tryptic treatment, the cell material of at least two culture dishes was obtained, and the number of cells determined by means of a CASY TT cell counter (150 μl capillary). By diluting the cell suspension with DMEM: Ham's F12, a cell density of 1×10⁶ cells/ml was adjusted. Of the thus obtained cell suspensions, 1 ml each was placed on a well of a 24-well plate so that each well was coated with 1×10⁶ cells. With the cell material of two confluently grown culture dishes, two 24-well plates could be coated. After 24 hours, the cells had grown on, and after another 24 hours' stimulation phase with a solution containing potassium ions (final concentration: 20 mM KCl), could be employed for the test.

B) Substrate solutions: For testing the influence of the inhibitors on CYP11B1, tritium-labeled deoxycortisol was employed as the substrate ([³H]-RSS=17-hydroxy-11-deoxycorticosterone, 41.9 Ci/mmol). For preparing a mixture of labeled and unlabeled substances, 38 μl of unlabeled deoxycortisol (0.5 mM in ethanol) and 41.6 μl of [1,2-³H(N)]deoxycortisol (1 mCi/ml, 52 Ci/mmol; NEN-Perkin-Elmer) in ethanol were diluted with 120.4 μl of ethanol. Of this solution, 2.5 μl was employed per sample, which corresponded to a final concentration of 500 nM in the test for a test volume of 500 μl.

In the substrate solutions for the examinations on CYP11B2, the corticosterone substrate solution (final concentration in the test: 500 nM) consisted of 38.4 μl of [1,2-³H(N)]corticosterone (1 mCi/ml, 76.5 Ci/mmol; NEN-Perkin-Elmer) in ethanol, 39.0 μl of unlabeled corticosterone solution (0.5 mM in ethanol) and 122.6 μl of ethanol. Deoxycorticosterone, which was also employed in a final concentration of 500 nM, was composed of 18 μl of [¹⁴C]-labeled deoxycorticosterone (60.0 mCi/mmol; 0.5 nCi/μl) in ethanol in admixture with 54 μl of unlabeled substance (0.5 mM in ethanol) and 228 μl of ethanol.

C) Inhibitor solutions: The concentrations required for the determination of the IC₅₀ values were adjusted by diluting the stock solution (10 mM) at 1:40 with ethanol. Of this solution, 5 μl each was added to the samples.

### D) Performance of the Tests:

Preincubation: The medium present was sucked off and replaced by 450 μl of DMEM: Ham's F12 in which the inhibitor was added in the corresponding concentration (final concentration of the inhibitor in the final volume (500 μl) of the test: 2.5 μM), followed by preincubation for 1 h.

Test start: The reaction was initiated by adding 50 μl of DMEM: Ham's F12 containing 2.5 μl of the respective substrate mix (final concentration of the substrate: 0.5 μM).

Then, the 24-well plate was stored in a CO₂ incubator at 37° C. and ⁵⁰% CO₂. The incubation time was 3 hours when deoxycorticosterone was used as the substrate, 24 Hours for corticosterone, and 48 hours for deoxycortisol.

Test stop: After elapse of the incubation times, the plates were briefly swung, and then the content of the wells was removed quantitatively if possible and inactivated by mixing with 1000 μl of dichloromethane in a 2 ml Eppendorf vessel. After 10 minutes of shaking, it was centrifuged for phase separation, and the upper, organic phase was transferred into a 1.5 ml Eppendorf vessel.

After evaporating the solvent over night under a hood, the residue was taken up in 10 μl of chloroform and applied to the center of the concentration zone of an HPTLC plate. The steroids were separated by developing twice with a mobile solvent composed of chloroform, methanol and water in a ratio of 300:20:1. In the case of deoxycortisol as the substrate, the separation was effected by HPLC over an RP18 column with the mobile solvent methanol:water 1.1 and a flow rate of 0.25 ml/min, the detection was effected by means of a Berthold Radiomonitor 509.

For detecting the steroids on the TLC, the radiation-exposed film was scanned in the phosphoimager FLA 3000 after two days.

The conversion for the substrate deoxycortisol after HPLC separation was calculated according to equation 4:

$$\% P = \frac{{A}_{Cortisone} + {A}_{Cortistone}}{[ {A}_{Cortisone} + {A}_{Cortisol} + {A}_{RSS} ]} \times 100 \qquad Equation 4$$

% P conversion rate (proportion of product to total steroid in %)
A area in [units·sec]
A_{Cortisol} area for cortisol
A_{Cortisone} area for cortisone
A_{RSS} area for deoxycortisol (RSS)

For the substrate deoxycorticosterone, the conversion was obtained in accordance with equation 3 (Ex. 5B).

For the substrate corticosterone, equation 5 was valid:

$$\% P = \frac{[ P S {L}_{18 OHB} + P S {L}_{Aldo} ] - 2 \times P S {L}_{HG}}{[ P S {L}_{B} + P S {L}_{18 OHB} + P S {L}_{Aldo} ] - 3 \times P S {L}_{HG}} \times 100 \qquad Equation 5$$

% P conversion rate (proportion of product to total steroid in %)
PSL phospho-stimulated luminescence (luminescence value)
PSL_{B} PSL for corticosterone (B)
PSL_{18OHB} PSL for 18-hydroxycorticosterone (18OHB)
PSL_{Aldo} PSL for aldosterone
PSL_{HG} PSL of the background

The percent inhibition caused by an inhibitor in the respectively employed concentration was calculated according to equation 2 (Ex. 5A).

The determination of IC₅₀ was effected as described in Example 5B.

### Example 13

### Test of Compound 50

Compound 50 was tested in V79 cells for inhibition of CYP11B1 and CYP11B2. Compound 50 inhibits human aldosterone synthase in the low nanomolar range and additionally shows a very weak inhibition of human CYP11B2. The substance is not only highly potent, but also very selective. Thus, substances of the class of 1,2-dihydroacenaphthylenes substituted in 3-position represent new leads which may lead to even more potent and at the same time highly selective CYP11B2 inhibitors.

TABLE 8
Inhibition data for the acenaphthene derivative 50

IC₅₀ (nM)^{a}

| | V79 11B1^{b} | V79 11B2^{c} | Selectivity |
|---|---|---|---|
| Compound | CYP11B1 | CYP11B2 | factor^{d} |
| 50 | 2452 | 10 | 245 |

^{a}Mean value of 4 determinations, standard error <20%.
^{b}Hamster fibroblasts which express human CYP11B1; deoxycorticosterone substrate, 100 nM.
^{c}Hamster fibroblasts which express human CYP11B2; deoxycorticosterone substrate, 100 nM.
^{d}IC₅₀ (CYP11B1)/IC₅₀ (CYP11B2).

1. Use of a compound having the structure of formula (I) 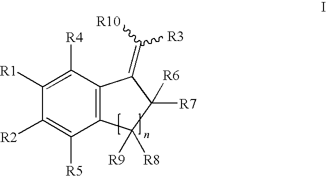
wherein
R¹ and R² are independently selected from H, halogen, CN, hydroxy, nitro, alkyl, alkoxy, alkylcarbonyl, alkylcarbonyloxy, alkylsulfinyl and alkylsulfonyl (the alkyl radicals being straight or branched-chain or cyclic, saturated or unsaturated, and optionally substituted with 1 to 3 radicals R¹²); aryl and heteroaryl radicals and their partially or completely saturated equivalents, optionally substituted with 1 to 3 radicals R¹²; aryloxy- and heteroaryloxy radicals, wherein aryl and heteroaryl have the above meanings, —COOR¹¹, —SO₃R¹¹, —CHO, —CHNR¹¹, —N(R¹¹)₂, —NHCOR¹¹ and —NHS(O)₂R¹¹;
R³ is selected from nitrogen-containing monocyclic or bicyclic heteroaryl radicals and their partially or completely saturated equivalents, optionally substituted with 1 to 3 radicals R¹² and comprising at least one nitrogen atom that is not bound to the methylidene carbon atom and not substituted;
R⁴, R⁵, R⁶, R⁷, R⁸, R⁹ and R¹⁰ are independently selected from H, halogen, CN, hydroxy, nitro, lower alkyl, lower alkoxy, (lower alkyl)carbonyl, (lower alkyl)carbonyloxy, (lower alkyl)carbonylamino, (lower alkyl)sulfonylamino, (lower alkyl)thio, (lower alkyl)sulfinyl and (lower alkyl)sulfonyl (the lower alkyl radicals being straight or branched-chain or cyclic, saturated or unsaturated, and optionally substituted with 1 to 3 radicals R¹²); —N(R¹¹)₂, —COOR¹¹ and —SO₃R¹¹; or
R⁸ or R⁹ together with R⁶ or R⁷ and/or with R⁸ or R⁹ of the neighboring carbon atom form one or two double bonds; or
R⁸ (and R⁹) together with R⁶ (and R⁷) or with R⁸ (and R⁹) of the neighboring carbon atom and the related carbon atoms form a saturated or unsaturated anellated aryl or heteroaryl ring, wherein the atoms of said anellated aryl or heteroaryl ring may be substituted with 1-3 radicals R¹²; or
R⁴ and R¹⁰ together form a methylene, ethylene or ethylidene bridge, wherein the atoms of the bridge may be substituted with one or two radicals R¹²; or
a ring atom in the ortho position of the heteroaryl radical of R³ forms a bond with R⁶ and/or R⁷ directly or through a methylene or methylidene bridge, wherein the bridge atom may be substituted with one or two radicals R¹²;
R¹¹ independently of the occurrence of other R¹¹ radicals is selected from H, lower alkyl (which may be straight or branched-chain or cyclic, saturated or unsaturated, and optionally substituted with 1 to 3 radicals R¹²) and aryl which may be substituted with 1 to 3 radicals R¹²;
R¹² independently of the occurrence of other R¹² radicals is selected from H, hydroxy, —CN, —COOH, —CHO, nitro, amino, mono- and bis-(lower alkyl)amino, lower alkyl, lower alkoxy, (lower alkyl)carbonyl, (lower alkyl)carbonyloxy, (lower alkyl)carbonylamino, (lower alkyl)thio, (lower alkyl)sulfinyl, (lower alkyl)sulfonyl, hydroxy(lower alkyl), hydroxy(lower alkoxy), hydroxy(lower alkyl)carbonyl, hydroxy(lower alkyl)carbonyloxy, hydroxy(lower alkyl)carbonylamino, hydroxy(lower alkyl)thio, hydroxy(lower alkyl)sulfinyl, hydroxy(lower alkyl)sulfonyl, mono- and bis(hydroxy(lower alkyl)amino and mono- and polyihalogenated (lower alkyl) (wherein the (lower alkyl) radicals may be straight or branched-chain or cyclic, saturated or unsaturated);
n is an integer of from 1 to 3;
or a pharmaceutically acceptable salt thereof
for the treatment of hypercortisolism, diabetes mellitus, heart insufficiency and myocardial fibrosis.

2. The use according to claim 1, wherein said compound of formula (I) is a compound of the following formulas (Ia) to (Ig): 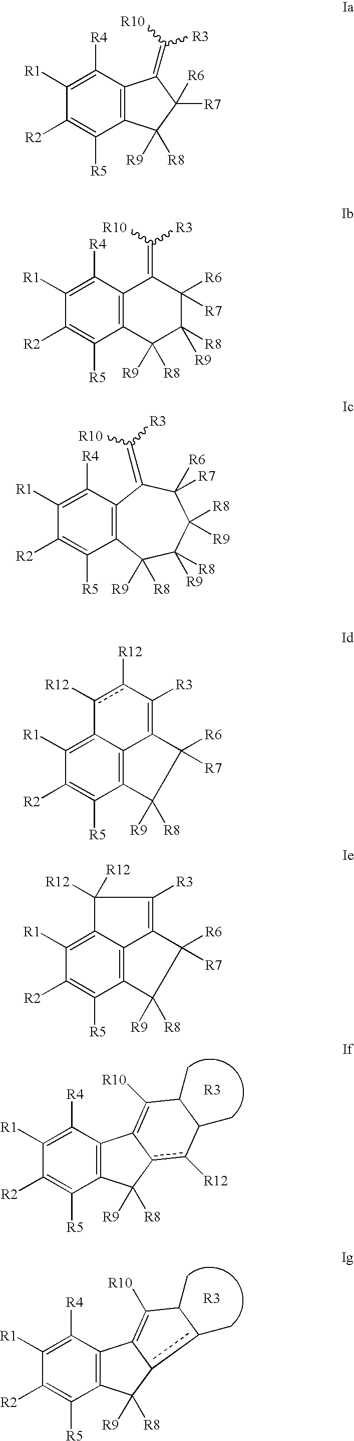
wherein all variables have the meanings given above, and [symbol] is either a single or a double bond; a compound of formula (Ia), (Ib), (Ic) or (Id) being particularly preferred.

3. The use according to claim 1, wherein in the compound of formulas (I) and (Ia) to (Ig):
(i) the alkyl radicals and alkoxy radicals are saturated or have one or more double and/or triple bonds, the straight or branched-chain alkyl radicals have, in particular, from 1 to 10 carbon atoms, more preferably from 1 to 6 carbon atoms, and the cyclic alkyl radicals are mono- or bicyclic alkyl radicals having from 3 to 15 carbon atoms, more preferably monocyclic alkyl radicals having from 3 to 8 carbon atoms;
(ii) aryl is a mono-, bi- and tricyclic aryl radical having from 3 to 18 ring atoms which may optionally be anellated with one or more saturated rings, especially is anthracenyl, dihydronaphthyl, fluorenyl, hydrindanyl, indanyl, indenyl, naphthyl, phenanthrenyl, phenyl or tetralinyl;
(iii) the heteroaryl radicals are mono- or bicyclic heteroaryl radicals having from 3 to 12 ring atoms preferably comprising from 1 to 5 heteroatoms selected from nitrogen, oxygen and sulfur, optionally anellated with one or more saturated rings; and/or
(iv) the lower alkyl radicals and lower alkoxy radicals are saturated or have a double or triple bond, the straight-chain ones having, in particular, from 1 to 6 carbon atoms, more preferably from 1 to 3 carbon atoms, and the cyclic ones having, in particular, from 3 to 8 carbon atoms; and/or
(v) the nitrogen-containing monocyclic or bicyclic heteroaryl radicals are selected from benzimidazolyl, benzothiazolyl, benzoxazolyl, quinazolinyl, quinolyl, quinoxalinyl, cinnolinyl, dihydroindolyl, dihydroisoindolyl, dihydropyranyl, dithiazolyl, homopiperidinyl, imidazolidinyl, imidazolinyl, imidazolyl, indazolyl, indolyl, isoquinolyl, isoindolyl, isothiazolidinyl, isothiazolyl, isoxazolidinyl, isoxazolyl, morpholinyl, oxadiazolyl, oxazolidinyl, oxazolyl, phthalazinyl, piperazinyl, piperidyl, pteridinyl, purinyl, pyrazolidinyl, pyrazinyl, pyrazolyl, pyrazolinyl, pyridazinyl, pyridyl, pyrimidyl, pyrrolidinyl, pyrrolidin-2-onyl, pyrrolinyl, pyrrolyl, tetrazinyl, tetrazolyl, tetrahydropyrrolyl, thiadiazolyl, thiazinyl, thiazolidinyl, thiazolyl, triazinyl and triazolyl; and/or
(vi) anellated aryl or heteroaryl rings are monocyclic rings with from 5 to 7 ring atoms which are anellated with the neighboring ring through two neighboring ring atoms, may be saturated or unsaturated and, as heteroaryl rings, comprise from 1 to 3 heteroatoms, preferably nitrogen, oxygen or sulfur atoms, more preferably being selected from cyclohexyl, cyclohexenyl, cyclopentyl, cyclopentenyl, benzyl, furanoyl, dihydropyranyl, pyranyl, pyrrolyl, imidazolyl, pyridyl and pyrimidyl.

4. The use according to one or more of claims 1, wherein in the compound of formulas (I) and (Ia) to (Ig):
(i) R¹ or R² are independently selected from hydrogen, halogen, CN, hydroxy, C₁₋₁₀ alkyl and C₁₋₁₀ alkoxy radicals, wherein said alkyl radicals or alkoxy radicals are straight or branched chain and may be substituted with 1 to 3 radicals R¹²; and/or
(ii) R³ is selected from nitrogen-containing monocyclic heteroaryl radicals with 5-10 ring atoms comprising 1 to 3 nitrogen atoms, especially selected from isoquinolyl, imidazolyl, oxazolyl, pyrazinyl, pyrazolyl, pyridyl, pyrimidyl, pyrrolyl, thiazolyl, triazinyl and triazoyl; and/or
(iii) R⁴, R⁵, R⁶, R⁷, R⁸, R⁹, R¹⁰ are independently selected from H, halogen, CN, hydroxy and C₁₋₆ alkyl and C₁₋₆ alkoxy radicals which may be substituted with 1 to 3 radicals R¹²; and/or
(iv) R¹² is selected from H, halogen, hydroxy, CN, C₁₋₃-alkyl and C₁₋₃-alkoxy; and/or
(v) n is 1 or 2.

5. The use according to claim 4, wherein in the compound of formulas (I) and (Ia) to (Ig), preferably in the compound of formulas (Ia) to (Ic):
(i) R¹ or R² is hydrogen;
(ii) the other of substituents R¹ or R² is selected from H, fluorine, chlorine, CN, hydroxy, C₁₋₃-alkyl and C₁₋₃-alkoxy;
(iii) R³ is selected from pyridyl, imidazolyl, isoquinolyl and pyrimidyl; and
(iv) R⁴, R⁵, R⁶, R⁷, R⁸, R⁹, R¹⁰ and R¹² are H.

6. The use according to claim 5, wherein in the compound of formula (Id):
(i) R¹ or R² is hydrogen;
(ii) the other of substituents R¹ or R² is selected from H, fluorine, chlorine, CN, hydroxy, C₁₋₃-alkyl and C₁₋₃-alkoxy;
(iii) R³ is selected from pyridyl, imidazolyl, isoquinolyl and pyrimidyl;
(iv) R⁴, R⁵, R⁶, R⁷, R⁸, R⁹ and R¹² are H; and
(v) [symbol] is a double bond.

7. The use according to claim 1, wherein said compound of formula (I) is:
E,Z-4-(5-chloro-1-indanylidenemethyl)-imidazole,
E,Z-4-(5-fluoro-1-indanylidenemethyl)-imidazole,
E,Z-4-(1,2,3,4-tetrahydronaphth-1-ylidenemethyl)-imidazole,
E,Z-4-(6-cyano-1,2,3,4-tetrahydronaphth-1-ylidenemethyl)-imidazole,
E,Z-4-(7-fluoro-1,2,3,4-tetrahydronaphth-1-ylidenemethyl)-imidazole,
E,Z-4-(7-chloro-1,2,3,4-tetrahydronaphth-1-ylidenemethyl)-imidazole,
E,Z-3-(1-indanylidenemethyl)-pyridine,
E,Z-3-(5-fluoro-1-indanylidenemethyl)-pyridine,
E,Z-3-(5-chloro-1-indanylidenemethyl)-pyridine,
E,Z-3-(4-fluoro-1-indanylidenemethyl)-pyridine,
E,Z-3-(4-chloro-1-indanylidenemethyl)-pyridine,
E,Z-3-(5-methoxy-1-indanylidenemethyl)-pyridine,
E,Z-3-(7-methoxy-1-indanylidenemethyl)-pyridine,
E,Z-3-(5-fluoro-1-indanylidenemethyl)-pyrimidine,
either as a mixture of isomers or one of the two isomers;
and especially
Z-4-(5-chloro-1-Indanylidenemethyl)-imidazole,
Z-4-(1,2,3,4-tetrahydronaphth-1-ylidenemethyl)-imidazole,
Z-4-(6-cyano-1,2,3,4-tetrahydronaphth-1-ylidenemethyl)-imidazole,
E-3-(1-indanylidenemethyl)-pyridine,
E-3-(5-fluoro-1-indanylidenemethyl)-pyridine,
E-3-(5-chloro-1-indanylidenemethyl)-pyridine,
E-3-(5-methoxy-1-indanylidenemethyl)-pyridine,
E-3-(4-fluoro-1-indanylidenemethyl)-pyridine,
E-3-(7-methoxy-1-indanylidenemethyl)-pyridine,
E-3-(5-fluoroindanylidenemethyl)-pyrimidine and 3-(1,2-dihydroacenaphthylen-3-yl)pyridine.

8. The use according to claim 1, wherein said compound of formula (I) is Z-4-(5-chloro-1-indanylidenemethyl)-imidazole.

9. A compound of formula (I) 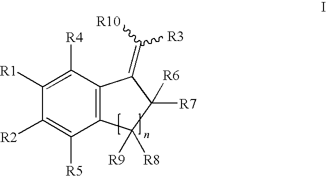
wherein
R¹, R², R³, R⁴, R⁵, R⁶, R⁷, R⁸, R⁹, R¹⁰, R¹¹ and R¹² have the meanings as stated in claim 1, with the proviso that:
(a) if n=1, R¹, R² and R⁴-R¹⁰ are hydrogen, then R³ is not 4-imidazolyl or 4-pyridyl;
(b) if n=2, R² and R⁴-R¹⁰ are hydrogen and R¹ is Cl or CN, then R³ is not 4-imidazolyl;
(c) if n=2, R¹ and R⁴-R¹⁰ are hydrogen and R² is CN, then R³ is not 4-imidazolyl;
(d) if n=2, R¹ and R⁴-R¹⁰ are hydrogen and R² is F, Cl, Br or CN, then R³ is not 4-imidazolyl;
(e) if n=2, R¹, R² and R⁴-R¹⁰ are hydrogen, then R³ is not 4-imidazolyl, 4-pyridyl, 4-methyl-3-pyridyl or 3-nitroimidazo[1,2-a]pyrid-2-yl;
(f) if n=1 or 2; three of the radicals R¹, R², R⁴ and R⁵ are independently hydrogen, C₁₋₄-alkyl, C₂₋₄-alkenyl, C₃₋₇-cycloalkyl, hydroxy, C₁₋₄-alkoxy, hydroxy-C₁₋₄-alkyl, halogen, trifluoromethyl, nitro or optionally substituted amino and the fourth radical of R¹, R², R⁴ and R⁵ is hydrogen, R⁶ is hydrogen, R⁷ is hydrogen or C₁₋₄-alkyl, R⁸ is hydrogen, C₁₋₄-alkyl, hydroxy or C₁₋₄-alkoxy, R⁹ and R¹⁰ are independently hydrogen or C₁₋₄-alkyl, then R³ is not 4-imidazolyl;
(g) if n=1 or 2, three of the radicals R¹, R², R⁴ and R⁵ are independently hydrogen, hydroxy, amino, halo-C₁₋₆-alkyl, C₁₋₆-alkyl, C₁₋₆-alkoxy or hydroxy-C₁₋₆-alkyl and the fourth radical of R¹, R², R⁴ and R⁵ is hydrogen, one of the radicals R⁶, R⁷, R⁸ and R⁹ is C₃₋₇-cycloalkyl, C₅₋₇-cycloalkenyl, C₃₋₇-cycloalkylmethyl or C₃₋₇-cycloalkenylmethyl, wherein the methyl radical may be substituted with one or two C₁₋₆-alkyl radicals, two of the radicals R⁶, R⁷, R⁸ and R⁹ are independently hydrogen, hydroxy, C₁₋₆-alkyl, halo-C₁₋₆-alkyl, C₁₋₆-alkoxy or hydroxy-C₁₋₆-alkyl, and the remaining radicals R⁶, R⁷, R⁸ and R⁹ are hydrogen, R¹⁰ is hydrogen or C₁₋₆-alkyl, then R³ is not 4-imidazolyl;
(h) if n=1, R¹ is hydrogen, hydroxy, alkoxy or alkylcarbonyloxy, R² is hydroxy, alkylcarbonyloxy or alkoxy, R⁴-R¹⁰ are hydrogen, then R³ is not 4-pyridyl;
(i) if n=2, R¹ is hydrogen, R² is hydroxy, C₁₋₄-alkoxy or C₁₋₄-alkylcarbonyloxy, R⁴-R⁹ are hydrogen, R¹⁰ is hydrogen or C₁₋₄-alkyl, then R³ is not 4-pyridyl;
(j) if n=1, R¹, R², R⁴, R¹, R⁸-R¹⁰ are hydrogen, R⁶ and R⁷ are both hydrogen or both methyl, then R³ is not 2-pyridyl;
(k) if n=2, R¹, R², R¹, R⁵ and R⁸-R¹⁰ are hydrogen, R⁶ and R⁷ are both methyl, then R³ is not 2-pyridyl;
(l) if n=2, R¹ is hydrogen, R² is hydrogen or methoxy, R⁴-R¹⁰ are hydrogen, then R³ is not 4-methyl-3-pyridyl;
or their pharmaceutically acceptable salts.

10. The compounds according to claim 9, wherein
(i) R1 or R2 are independently selected from hydrogen, halogen, CN, hydroxy, C1 10 alkyl and C1-10 alkoxy radicals, wherein said alkyl radicals or alkoxy radicals are straight or branched chain and may be substituted with 1 to 3 radicals R12; and/or
(ii) R3 is selected from nitrogen-containing monocyclic heteroaryl radicals with 5-10 ring atoms comprising 1 to 3 nitrogen atoms, especially selected from isoquinolyl, imidazolyl, oxazolyl, pyrazinyl, pyrazolyl, pyridyl, pyrimidyl, pyrrolyl, thiazolyl, triazinyl and triazoyl; and/or
(iii) R4, R5, R6, R7, R8, R9, R10 are independently selected from H, halogen, CN, hydroxy and C1-6 alkyl and C1-6 alkoxy radicals which may be substituted with 1 to 3 radicals R12; and/or
(iv) R12 is selected from H, halogen, hydroxy, CN, C1-3-alkyl and C1-3-alkoxy; and/or
(v) n is 1 or 2.

11. A process for synthesizing the compounds according to claim 9, comprising the conversion of compound (II): 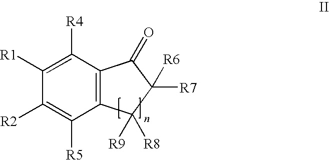
to the corresponding alcohol, followed by a Wittig reaction with compound (III) 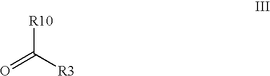
wherein the variables have the meaning as stated in claim 9, and functional groups in R¹-R¹⁰ may optionally be provided with suitable protective groups.

12. A pharmaceutical composition containing a compound as defined in claim 9.

13. The pharmaceutical composition according to claim 12 suitable for the therapy of heart insufficiency, myocardial fibrosis, hypercortisolism or diabetes mellitus in mammals and humans.

14. Use of the compounds as defined in claim 9 for the selective inhibition of mammal P450 oxygenases, for the inhibition of human or mammal aldosterone synthase or steroid-11β-hydroxylase, especially for the inhibition of human steroid-11β-hydroxylase CYP11B1 or aldosterone synthase CYP11B2, especially for the selective inhibition of CYP11B2 while human CYP11B1 is little affected.

15. The use according to claim 9, wherein said compounds are employed:
(i) as individual compounds; or
(ii) as components of mixtures containing one or a combination of two or more of the compounds of claim 9; or
(iii) in combination with further pharmacologically active compounds.

16. A process for the prevention, deceleration of the progress or therapy of diabetes mellitus, hypercortisolism, hypertension, congestive heart failure, kidney failure, especially chronic kidney failure, restenosis, atherosclerosis, nephropathy, coronary heart diseases, increased formation of collagen, fibrosis, respectively associated or not with occurrence of hypertension in an individual, comprising the administration of a compound as defined in one or more of claim 1 to said individual.
